(19) United States
(12) Patent Application Publication
(10) Pub. No.: US 2019/0070113 A1
(43) Pub. Date: Mar. 7, 2019

(54) Treating Ephrin Receptor A2 (Epha2) Positive Cancer with Targeted Docetaxel-Generating Nano-Liposome Compositions

(71) Applicant: Merrimack Pharmaceuticals, Inc., Cambridge, MA (US)

(72) Inventors: Daryl C. Drummond, Lincoln, MA (US); Walid Kamoun, Arlington, MA (US)

(73) Assignee: Merrimack Pharmaceuticals, Inc., Cambridge, MA (US)

(21) Appl. No.: 16/085,508

(22) PCT Filed: Mar. 16, 2017
(86) PCT No.: PCT/US2017/022629

§ 371 Date: Sep. 14, 2018

Related U.S. Application Data
(60) Provisional application No. 62/464,574, filed on Feb. 28, 2017.
(60) Provisional application No. 62/419,047, filed on Nov. 8, 2016.
(60) Provisional application No. 62/322,991, filed on Apr. 15, 2016.
(60) Provisional application No. 62/309,240, filed on Mar. 16, 2016.

(51) Int. Cl. A61K 9/127; A61K 31/337; A61K 31/7068; A61K 31/555; A61K 47/69; A61K 47/68; A61P 35/00

(52) U.S. Cl. CPC A61K 9/1271; A61K 31/337; A61K 31/7068; A61P 35/00; A61K 47/6913; A61K 47/6859; A61K 31/555

(57) ABSTRACT

EphA2 targeted doxorubicin generating nano-liposomes are useful in the treatment of cancer overexpressing EphA2, alone or in combination with chemotherapeutic agents such as gemcitabine or carboplatin.

### CROSS-REFERENCE

This patent application claims priority to each of the following pending U.S. provisional patent applications, each incorporated herein by reference is their entirety: 62/309,240 (filed Mar. 16, 2016), 62/322,991 (filed Apr. 15, 2016), 62/419,047 (filed Nov. 8, 2016) and 62/464,574 (filed Feb. 28, 2017).

### SEQUENCE LISTING

Incorporated by reference in its entirety is a computer-readable sequence listing submitted concurrently herewith and identified as follows: One 48.0 KB ASCII (Text) file named “1108sequence_ST25.txt.”

### TECHNICAL FIELD

This disclosure relates to docetaxel-generating nano-liposomes that bind to Ephrin receptor A2 (EphA2), useful in the treatment of EphA2-positive cancer.

### BACKGROUND

Ephrins receptors are cell to cell adhesion molecules that mediate signaling and are implicated in neuronal repulsion, cell migration and angiogenesis. EphA2 is part of the Ephrin family of cell-cell junction proteins highly overexpressed in several solid tumors. Ephrin receptor A2 (EphA2) is overexpressed in several solid tumors including prostate, pancreatic, ovarian, gastric and lung cancer, and is associated with poor prognosis in certain cancer conditions. The Eph receptors are comprised of a large family of tyrosine kinase receptors divided into two groups (A and B) based upon homology of the N-terminal ligand binding domain. The Eph receptors are involved several key signaling pathways that control cell growth, migration and differentiation. These receptors are unique in that their ligands bind to the surface of neighboring cells. The Eph receptors and their ligands display specific patterns of expression during development. For example the EphA2 receptor is expressed in the nervous system during embryonic development and also on the surface of proliferating epithelial cells in adults. EphA2 also plays an important role in angiogenesis and tumor vascularization, mediated through the ligand ephrin A1. In addition, EphA2 is overexpressed in a variety of human epithelial tumors including breast, colon, ovarian, prostate and pancreatic carcinomas. Expression of EphA2 can also be detected in tumor blood vessels as well.

Pancreatic cancer remains one of the deadliest cancers with survival described in number of months and weeks. Recent advances in the treatment of pancreatic cancer led to the recent approval of a liposomal irinotecan (ONIVYDE® (irinotecan liposome injection), previously MM-398).

### SUMMARY

We developed novel EphA2-targeted nanoliposomal docetaxel-generating molecules, including the EphA2-targeted, docetaxel-generating immunoliposomes 46scFv-ILs-DTXp3 and 46scFv-ILs-DTXp6, and evaluated activity of various therapies in various patient derived xenograft (PDX) models of cancer as a monotherapy, as well as in combination with gemcitabine. Additionally, we tested the predictive potential of key biomarkers that are linked to the 46scFv-ILs-DTXp3 mechanism of action.

We have discovered the use of novel EphA2 targeted docetaxel-generating nanoliposomes in the treatment of EphA2 positive tumors (including pancreatic cancer tumors), alone and in combination with certain chemotherapeutic agents such as gemcitabine. The discovery is based in part on an evaluation of an EphA2 targeted docetaxel-generating nanoliposome in certain patient derived pancreatic cancer xenograph models. The EphA2 targeted docetaxel-generating nanoliposome can be administered in combination with gemcitabine.

Several PDX models were screened for the expression of EphA2 (46scFv-ILs-DTXp3 target), CD31 (blood vessels), Massons Trichrome (fibrosis), CA XI (hypoxia), and E-Cadherin (adhesion molecule that can potentially inhibit target engagement). Eight EphA2+PDX models were used to evaluate the activity of 46scFv-ILs-DTXp3 and compare it to clinically relevant agents including nab-paclitaxel, liposomal irinotecan, oxaliplatin, and gemcitabine. We also tested the combination potential of 46scFv-ILs-DTXp3 and gemcitabine.

The representative compound 46scFv-ILs-DTXp3 was able to statistically significantly control tumor growth in all tested models with tumor regression in more than 85% of the models. When compared with standard of care agents in tumor models, 46scFv-ILs-DTXp3 demonstrated greater activity to nab-paclitaxel in 80% (4/5), gemcitabine in 100% (5/5), and oxaliplatin in100% (5/5), and liposomal irinotecan in 80% (4/5). Gemcitabine is currently considered a standard of care in pancreatic cancer in combination with nab-paclitaxel, thus we conducted a study to evaluate the potential combination benefits of gemcitabine with 46scFv-ILs-DTXp3. The combination of suboptimal doses of 46scFv-ILs-DTXp3 and gemcitabine led to significant tumor growth control which was greater to either arm alone. Additionally, at equitoxic dosing of 50% maximum tolerated dose, 46scFv-ILs-DTXp3+gemcitabine showed greater effect than ABRAXANE (paclitaxel protein-bound particles for injectable suspension)+gemcitabine. Although we have excluded EphA2 negative models from these studies, biomarker analysis showed that 46scFv-ILs-DTXp3 effects are not correlated with the EphA2 expression level, suggesting that a low level EphA2 might be sufficient to mediate activity and that liposome delivery might be the rate limiting step. In conclusion, we found that 46scFv-ILs-DTXp3 is highly active in several patient derived models of pancreatic cancer and that it was equal or greater to most standard of care agents.

### BRIEF DESCRIPTION OF THE DRAWINGS

FIG. 1A is a schematic of a docetaxel-generating liposome comprising a EphA2 binding moiety (anti-EphA2 scFv PEG-DSPE).

FIG. 1B is a schematic showing the processes of docetaxel prodrug loading into a liposome comprising sucrose octasulfate (SOS) as a trapping agent, and the process of docetaxel generation. The insolubility of the salt in the liposome interior when combined with a low pH environment can stabilize the prodrug to reduce or prevent premature conversion to the active docetaxel.

FIG. 2A is a chemical reaction scheme for the synthesis of certain docetaxel prodrugs.

FIG. 2B is a chart showing selected examples of docetaxel prodrugs.

FIG. 2C is a reaction scheme showing the synthesis of PEG-DSG-E.

FIG. 3A is a schematic showing hydrolysis profiles at 37 deg C. for preferred docetaxel prodrugs. The hydrolysis profile can be obtained using the method of Example 11.

FIG. 3B is a hydrolysis profile for a certain docetaxel prodrug.

FIG. 3C is a hydrolysis profile for a certain docetaxel prodrug.

FIG. 3D is a hydrolysis profile for a certain docetaxel prodrug.

FIG. 3E is a hydrolysis profile for a certain docetaxel prodrug.

FIG. 4A is an amino acid sequence and corresponding encoding DNA sequence for the scFv EphA2 binding moiety in the 46scFv-ILs-DTXp3 docetaxel-generating liposome, used in Examples 2-9.

FIG. 4B shows various CDR sequences useful in EphA2 binding moieties that can be used to prepare EphA2-targeted docetaxel-generating liposomes.

FIG. 4C is an amino acid sequence and corresponding encoding DNA sequence for the scFv that can be used to prepare EphA2-targeted docetaxel-generating liposomes. The DNA sequence further encodes an N-terminal leader sequence that is cleaved off by mammalian (e.g., human or rodent) cells expressing the encoded scFv.

FIG. 4D is an amino acid sequence and corresponding encoding DNA sequence for the scFv that can be used to prepare EphA2-targeted docetaxel-generating liposomes. The DNA sequence further encodes an N-terminal leader sequence that is cleaved off by mammalian (e.g., human or rodent) cells expressing the encoded scFv.

FIG. 4E is an amino acid sequence and corresponding encoding DNA sequence for the scFv that can be used to prepare EphA2-targeted docetaxel-generating liposomes. The DNA sequence further encodes an N-terminal leader sequence that is cleaved off by mammalian (e.g., human or rodent) cells expressing the encoded scFv.

FIG. 4F is an amino acid sequence and corresponding encoding DNA sequence for the scFv that can be used to prepare EphA2-targeted docetaxel-generating liposomes. The DNA sequence further encodes an N-terminal leader sequence that is cleaved off by mammalian (e.g., human or rodent) cells expressing the encoded scFv.

FIG. 4G is an amino acid sequence and corresponding encoding DNA sequence for the scFv that can be used to prepare EphA2-targeted docetaxel-generating liposomes. The DNA sequence further encodes an N-terminal leader sequence that is cleaved off by mammalian (e.g., human or rodent) cells expressing the encoded scFv.

FIG. 4H is an amino acid sequence and corresponding encoding DNA sequence for the scFv that can be used to prepare EphA2-targeted docetaxel-generating liposomes. The DNA sequence further encodes an N-terminal leader sequence that is cleaved off by mammalian (e.g., human or rodent) cells expressing the encoded scFv.

FIG. 4I is an amino acid sequence and corresponding encoding DNA sequence for the scFv that can be used to prepare EphA2-targeted docetaxel-generating liposomes. The DNA sequence further encodes an N-terminal leader sequence that is cleaved off by mammalian (e.g., human or rodent) cells expressing the encoded scFv.

FIG. 4J is an amino acid sequence used in Example 4, and a corresponding encoding DNA sequence.

FIG. 5 is a graph showing tumor growth curves for model #12424 comparing 46scFv-ILs-DTXp3 to standard of care agents.

FIG. 6 is a graph showing time to regrowth for model #12424 comparing 46scFv-ILs-DTXp3 to standard of care agents.

FIG. 7 is a graph showing maximal response to drug for model #12424 comparing 46scFv-ILs-DTXp3 to standard of care agents.

FIG. 8 is a graph showing tumor growth curves for model #14244 comparing 46scFv-ILs-DTXp3 to standard of care agents.

FIG. 9 is a graph showing time to regrowth for model #14244 comparing 46scFv-ILs-DTXp3to standard of care agents.

FIG. 10 is a graph showing maximal response to drug for model #14244 comparing 46scFv-ILs-DTXp3 to standard of care agents.

FIG. 11 is a graph showing tumor growth curves for model #15010 comparing 46scFv-ILs-DTXp3 to standard of care agents.

FIG. 12 is a graph showing time to regrowth for model #15010 comparing 46scFv-ILs-DTXp3 to standard of care agents.

FIG. 13 is a graph showing maximal response to drug for model #15010 comparing 46scFv-ILs-DTXp3 to standard of care agents.

FIG. 14 is a graph showing tumor growth curves for model #14312 comparing nab-Paclitaxel to 46scFv-ILs-DTXp3.

FIG. 15 is a graph showing time to regrowth for model #14312 comparing nab-Paclitaxel to 46scFv-ILs-DTXp3.

FIG. 16 is a graph showing maximal response to drug for model #14312 comparing nab-Paclitaxel to 46scFv-ILs-DTXp3.

FIG. 17 is a graph showing tumor growth curves for model #12424 comparing nab-Paclitaxel to 46scFv-ILs-DTXp3.

FIG. 18 is a graph showing time to regrowth for model #12424 comparing nab-Paclitaxel to 46scFv-ILs-DTXp3.

FIG. 19 is a graph showing maximal response to drug for model #12424 comparing nab-Paclitaxel to 46scFv-ILs-DTXp3.

FIG. 20 is a graph showing tumor growth curves for model #15010 comparing nab-Paclitaxel to 46scFv-ILs-DTXp3.

FIG. 21 is a graph showing time to regrowth for model #15010 comparing nab-Paclitaxel to 46scFv-ILs-DTXp3.

FIG. 22 is a graph showing maximal response to drug for model #15010 comparing nab-paclitaxel to 46scFv-ILs-DTXp3.

FIG. 23 is a graph showing tumor growth curves for model #14244 comparing nab-Paclitaxel to 46scFv-ILs-DTXp3.

FIG. 24 is a graph showing time to regrowth for model #14244 comparing nab-Paclitaxel to 46scFv-s-DTXp3.

FIG. 25 is a graph showing maximal response to drug for model #14244 comparing nab-46scFv-s-DTXp3.

FIG. 26 is a graph showing tumor growth curves for model #14244 comparing Gemcitabine+46scFv-ILs-DTXp3 to Gemcitabine+nab-Paclitaxel.

FIG. 27 is a graph showing time to regrowth for model #14244 comparing Gemcitabine+46scFv-ILs-DTXp3 to Gemcitabine+nab-Paclitaxel.

FIG. 28 is a graph showing maximal response to drug comparing combination therapy of Gemcitabine+46scFv-ILs-DTXp3 to Gemcitabine+nab-Paclitaxel in model #14244.

FIG. 29 is a graph showing tolerability of 46scFv-ILs-DTXp3 in combination with carboplatin at 63 mg/kg with different combination scheduling schemes.

FIG. 30 is a graph showing tolerability of 46scFv-ILs-DTXp3 in combination with carboplatin at 72 mg/kg with different combination scheduling schemes.

FIG. 31 is a graph showing tolerability of 46scFv-ILs-DTXp3 in combination with carboplatin at 84 mg/kg with different combination scheduling schemes.

FIG. 32 is a graph showing tolerability of 46scFv-ILs-DTXp3 in combination with gemcitabine at 162 mg/kg with different combination scheduling schemes.

FIG. 33 is a graph showing tolerability of 46scFv-ILs-DTXp3 in combination with gemcitabine at 214 mg/kg with different combination scheduling schemes.

FIG. 34 is a graph showing tolerability of 46scFv-ILs-DTXp3 in combination with gemcitabine at 292 mg/kg with different combination scheduling schemes.

FIGS. 35-A-D are graphs showing effects of 46scFv-ILs-DTXp3 in combination with gemcitabine in tumor models BL-0382, BL-0293, and BL-0440.

FIGS. 36A-C are graphs showing effects of 46scFv-ILs-DTXp3 in combination with carboplatin in an ovarian tumor model.

### DETAILED DESCRIPTION

EphA2-targeted nanoliposomes can be used to deliver docetaxel (e.g., as an encapsulated docetaxel prodrug) to a cancer cell and/or tumor, leveraging organ specificity through the enhanced permeability and retention effect and cellular specificity through EphA2 targeting.

“EphA2” refers to Ephrin type-A receptor 2, also referred to as “epithelial cell kinase (ECK),” a receptor tyrosine kinase that can bind and be activated by Ephrin-A ligands. The term “EphA2” can refer to any naturally occurring isoforms of EphA2. The amino acid sequence of human EphA2 is recorded as GenBank Accession No. NP_004422.2.

As used herein, “EphA2 positive” refers to a cancer cell having at least about 3,000 EphA2 receptors per cell (or patient with a tumor comprising such a cancer cell). EphA2 positive cells can specifically bind Eph-A2 targeted liposomes per cell. In particular, EphA2 targeted liposomes can specifically bind to EphA2 positive cancer cells having at least about 3,000 or more EphA2 receptors per cell.

As used herein, non-targeted liposomes can be designated as “Ls” or “NT-Ls.” Ls (or NT-Ls) can refer to non-targeted liposomes with or without a docetaxel prodrug. “Ls-DTX′” refers to liposomes containing any suitable docetaxel prodrug, including equivalent or alternative embodiments to those docetaxel prodrugs disclosed herein. “NT-Ls-DTX” refers to liposomes without a targeting moiety that encapsulate any suitable docetaxel prodrug, including equivalent or alternative embodiments to those docetaxel prodrugs disclosed herein. Examples of non-targeted liposomes including a particular docetaxel prodrug can be specified in the format “Ls-DTXp[y]” or “NT-DTXp[y]” where [y] refers to a particular compound number specified herein. For example, unless otherwise indicated, Ls-DTXp1 is a liposome containing the docetaxel prodrug of compound 1 herein, without an antibody targeting moiety.

As used herein, targeted immunoliposomes can be designated as “ILs.” Recitation of “ILs-DTXp” refers to any embodiments or variations of the targeted docetaxel-generating immunoliposomes comprising a targeting moiety, such as a scFv. The ILs disclosed herein refer to immunoliposomes comprising a moiety for binding a biological epitope, such as an epitope-binding scFv portion of the immunoliposome. Unless otherwise indicated, ILs recited herein refer to EphA2 binding immunoliposomes (alternatively referred to as “EphA2-ILs”). The term “EphA2-ILs” refers herein to immunoliposomes enabled by the present disclosure with a moiety targeted to bind to EphA2. ILs include EphA2-ILs having a moiety that binds to EphA2 (e.g., using any scFv sequences that bind EphA2). Preferred targeted docetaxel-generating immunoliposomes include ILs-DTXp3, ILs-DTXp4, and ILs-DTXp6. Absent indication to the contrary, these include immunoliposomes with an EphA2 binding moiety and encapsulating docetaxel prodrugs of compound 3, compound 4 or compound 6 (respectively). EphA2-ILs can refer to and include immunoliposomes with or without a docetaxel prodrug (e.g., immunoliposomes encapsulating a trapping agent such as sucrose octasulfate without a docetaxel prodrug).

The abbreviation format “[x]scFv-ILs-DTXp[y]” is used herein to describe examples of immune-liposomes (“ILs”) that include a scFv “targeting” moiety having the amino acid sequence specified in a particular SEQ ID NO:[x], attached to a liposome encapsulating or otherwise containing a docetaxel prodrug (“DTXp”) having a particular Compound number ([y]) specified herein. Unless otherwise indicated, the scFv sequences for targeted ILs can bind to the EphA2 target.

The term “NT-Ls” refers to non-targeted liposomes enabled by this disclosure without a targeting moiety. The term “NT-LS-DTXp3” refers to a non-targeted liposomes enabled by this disclosure encapsulating a docetaxel prodrug (“DTX′”).

As used herein, the term “mpk” refers to mg per kg in a dose administered to an animal.

Preferably, the immunoliposomes (ILs) or non-targeted liposomes (Ls or NT-LS) comprise a suitable amount of PEG (i.e., PEGylated) attached to one or more components of the liposome vesicle to provide a desired plasma half-life upon administration.

In one embodiment, the invention is a method of treating a cancer comprising administering a therapeutically effective amount of an EphA2-targeted docetaxel-generating liposome comprising a docetaxel prodrug encapsulated within a lipid vesicle comprising one or more lipids, a PEG derivative and an EphA2 binding moiety on the outside of the lipid vesicle.

In some embodiments, the method further comprises administering the EphA2-targeted docetaxel-generating liposome in combination with gemcitabine. In some embodiments, the method further comprises administering the EphA2-targeted docetaxel-generating liposome in combination with carboplatin.

In some embodiments, the EphA2-targeted docetaxel-generating liposome is 46scFv-ILs-DTXp3 or 46scFv-ILs-DTXp6. In some embodiments, the EphA2-targeted docetaxel-generating liposome is 46scFv-ILs-DTXp3.

In some embodiments, the cancer is bladder cancer. In some embodiments, the cancer is a sarcoma cancer.

In one embodiment, the invention is a method of treating cancer in a human patient, the method comprising administering a therapeutically effective amount of the EphA2-targeted docetaxel-generating liposome ILs-DTXp3 or ILs-DTXp6 to the human patient.

In some embodiments, the liposome comprises sphingomyelin and cholesterol at a 3:2 molar ratio, and 5-7 mol % PEG-DSG.

In one embodiment, the invention is a use of a EphA2-targeted docetaxel-generating liposome ILs-DTXp3 or ILs-DTXp6 to the human patient to treat a sarcoma cancer or bladder cancer in a human patient, the use comprising administering a therapeutically effective amount of the EphA2-targeted docetaxel-generating liposome ILs-DTXp1 or ILs-DTXp3 to the human patient.

In some embodiments, the cancer comprises cancer cells expressing an average of at least 3000 EphA2 receptors per cell. In some embodiments, the cancer comprises a cancer cell expressing an average of at least 17500 EphA2 receptors per cell. In some embodiments, the cancer comprises a cancer cell expressing an average of at least 100,000 EphA2 receptors per cell.

In some embodiments, the liposome comprises sphingomyelin, cholesterol and PEG-DSG at a mole ratio of 3:2:0.03.

In some embodiments, the liposome encapsulates a docetaxel prodrug of Compound 3, Compound 4 or Compound 6. In some embodiments, the liposome encapsulates a sucrose octasulfate salt of Compound 3, Compound 4 or Compound 6.

In some embodiments, the cancer is an EphA2 overexpressing cancer.

In some embodiments, the cancer is selected from the group consisting of bladder or urothelial carcinoma, gastric, gastroesophageal junction or esophageal carcinoma (G/GEJ/E), squamous cell carcinoma of the head and neck (SCCHN), ovarian cancer, pancreatic ductal adenocarcinoma (PDAC), prostate adenocarcinoma (PAC), non-small cell lung cancer (NSCLC), small cell lung cancer (SCLC), triple negative breast cancer (TNBC), endometrial carcinoma and soft tissue sarcoma subtypes except GIST, desmoid tumors and pleomorphic rhabdomyosarcoma.

### EphA2-Targeted Liposomes for Delivery of Docetaxel

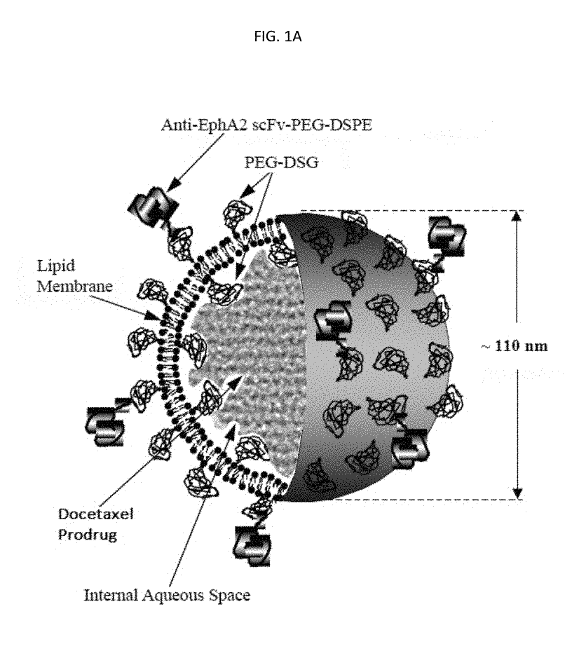

FIG. 1A is a schematic showing the structure of a PEGylated EphA2 targeted, nano-sized immunoliposome (nanoliposome) encapsulating a docetaxel prodrug (e.g., having a liposome size on the order of about 100 nm). The immunoliposome can include an Ephrin A2 (EphA2) targeted moiety, such as a scFv, bound to the liposome (e.g., through a covalently bound PEG-DSPE moiety). The PEGylated EphA2 targeted liposome encapsulating a docetaxel prodrug can be created by covalently conjugating single chain Fv (scFv) antibody fragments that recognize the EphA2 receptor to pegylated liposomes, containing docetaxel in the form of a prodrug described herein, resulting in an immunoliposomal drug product (FIG. 1A). In one particular example of a PEGylated EphA2 targeted liposome encapsulating a docetaxel prodrug (herein designated “EphA2-ILs-DTX”), the lipid membrane can be composed of egg sphingomyelin, cholesterol, and 1,2-distearoyl-sn-glyceryl methoxypolyethylene glycol ether (PEG-DSG). The nanoliposomes can be dispersed in an aqueous buffered solution, such as a sterile pharmaceutical composition formulated for parenteral administration to a human.

The EphA2 targeted nanoliposome of FIG. 1A is preferably a unilamellar lipid bilayer vesicle, approximately 110 nm in diameter, which encapsulates an aqueous space which contains a compound of disclosed herein in a gelated or precipitated state, as sucrosofate (sucrose octasulfate) salt. Example 1 describes methods of preparing a PEGylated EphA2 targeted liposome encapsulating a docetaxel prodrug.

The docetaxel prodrug can be stabilized in the liposomal interior during storage and while the intact liposome is in the general circulation, but is hydrolyzed rapidly (e.g., t½=˜10 h) to the active docetaxel upon release from the liposome and entering the environment of the circulating blood. FIG. 1B is a depiction of docetaxel nanogenerator with a docetaxel prodrug compound as disclosed herein. A docetaxel prodrug can be loaded at mildly acidic pH and entrapped in the acidic interior of liposomes, using an electrochemical gradient where it is stabilized in a non-soluble form. Upon release from the liposome, the docetaxel prodrug is subsequently converted to active docetaxel by simple base-mediated hydrolysis at neutral pH.

### Docetaxel Prodrug Compounds

The PEGylated EphA2 targeted liposome encapsulating a docetaxel prodrug can encapsulate one or more suitable docetaxel prodrugs. Preferably, the docetaxel prodrug comprises a weak base such as tertiary amine introduced to the 2′ or 7 position hydroxyl group of docetaxel through ester bond to form a docetaxel prodrug. Preferred 2′-docetaxel prodrugs suitable for loading into a liposome are characterized by comparatively high stability at acidic pH but convert to docetaxel at physiological pH through enzyme-independent hydrolysis.

As shown in FIG. 1B, the chemical environment of the 2′-ester bond can be tuned systematically to obtain docetaxel prodrugs that are stable at relatively low pH but will release free docetaxel rapidly at physiologic pH through hydrolysis. Docetaxel prodrugs are loaded into liposome at relatively low pH by forming stable complexes with trapping agents such as polysulfated polyols, for example, sucrose octasulfate. The trapping agent sucrose octasulfate can be included in the liposome interior, as a solution of its amine salt, such as diethylamine salt (DEA-SOS), or triethylamine salt (TEA-SOS). The use of amine salts of the trapping agents helps to create a transmembrane ion gradient that aids the prodrug loading into the liposome and also to maintain the acidic intraliposomal environment favorable for keeping the prodrug from premature conversion to docetaxel before the prodrug-loaded liposome reaches its anatomical target. Encapsulation of docetaxel prodrugs inside liposome in such a way allows the practical application of pH triggered release of docetaxel upon release from the liposome within the body of a patient. Thus, the liposome that encapsulates docetaxel-prodrug can be called docetaxel nanogenerator.

Preferably, the docetaxel prodrug is a compound of formula (I), including pharmaceutically acceptable salts thereof, where R1 and R2 are selected to provide desired liposome loading and stability properties, as well as desired docetaxel generation (e.g., as measured by the hydrolysis profile at various pH values, as disclosed herein). The docetaxel prodrug (DTX′) compounds can form a pharmaceutically acceptable salt within the liposome (e.g., a salt with a suitable trapping agent such as a sulfonated polyol). In some examples, the compounds of formula (I) where R1 and R2 are independently H or lower alkyl (preferably C₁-C₄ linear or branched alkyl, most preferably C₂ or C₃), and n is an integer (preferably 1-4, most preferably 2-3).

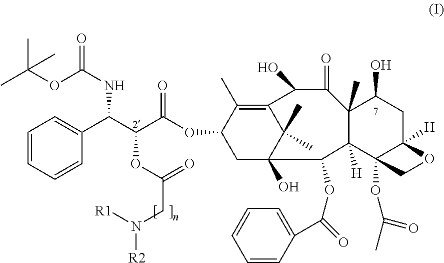

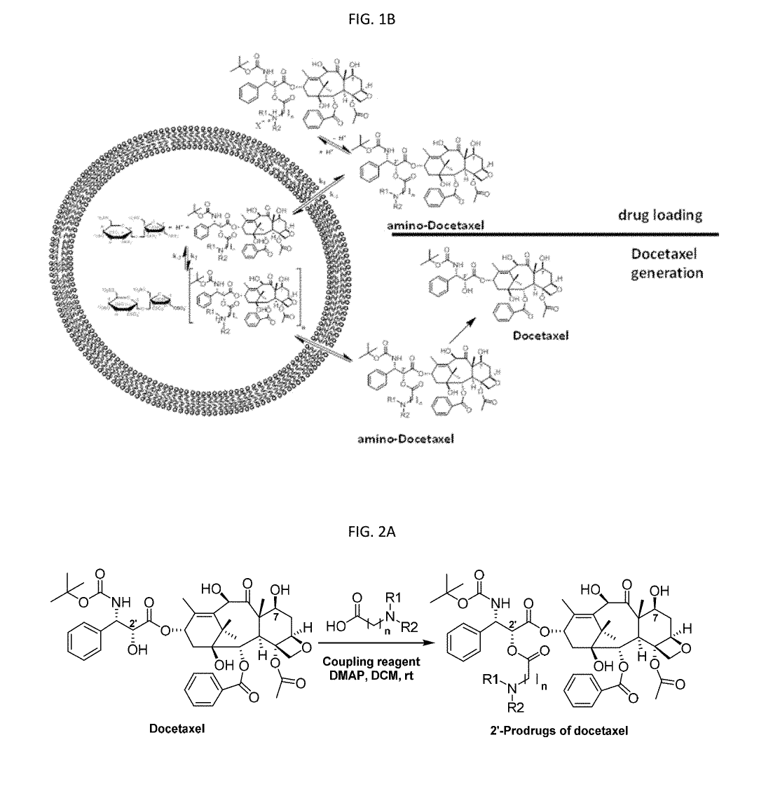

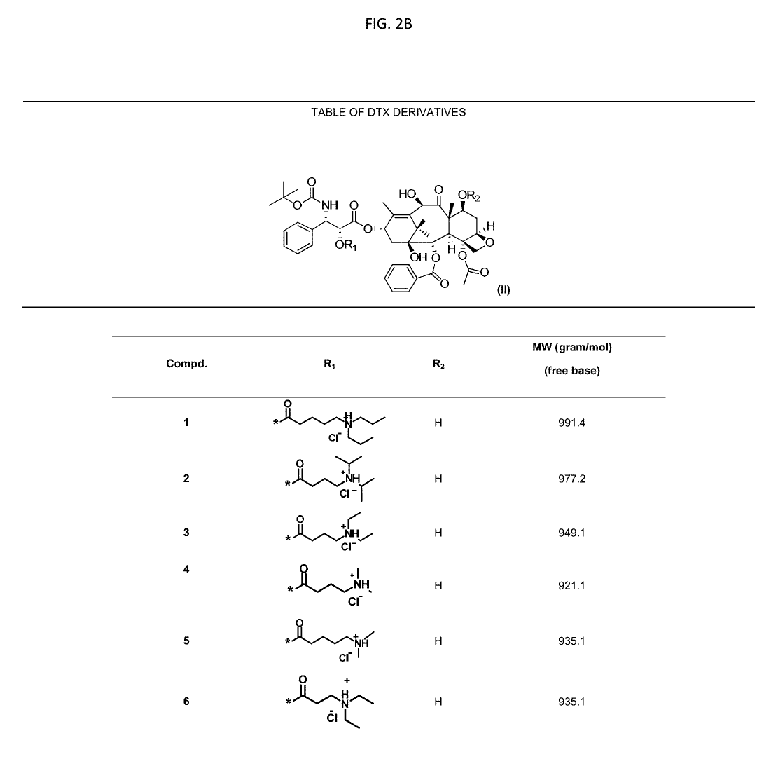

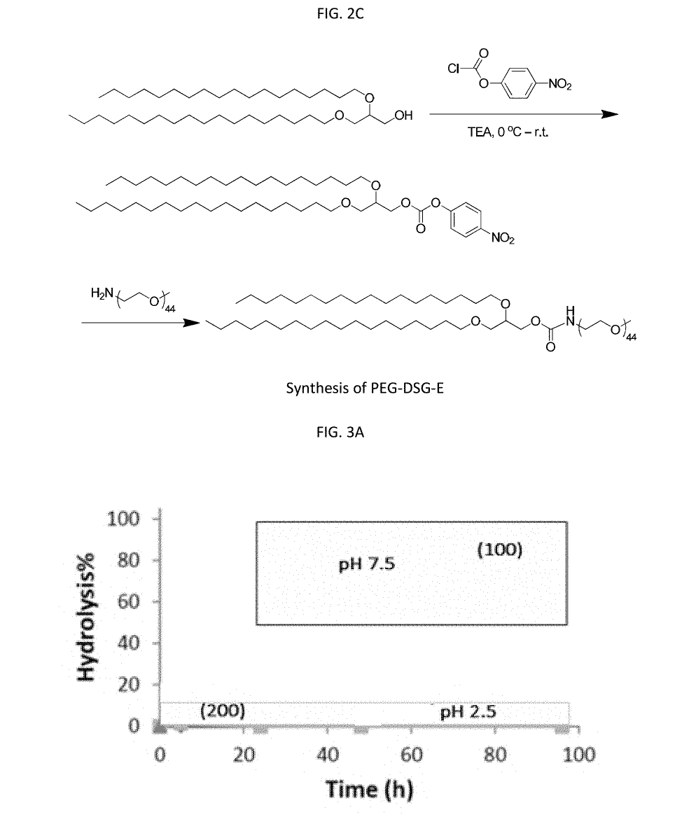

The docetaxel prodrugs, including compounds of Formula (I), can be prepared using the reaction Scheme in FIG. 2A. Two specific preparations of docetaxel prodrugs are described in Example 10A (Compound 3) and Example 10B (Compound 4). Other examples of docetaxel prodrugs include 2′-(2-(N,N′-diethylamino)propionyl)-docetaxel or 7-(2-(N,N′-diethylamino)propionyl)-docetaxel.

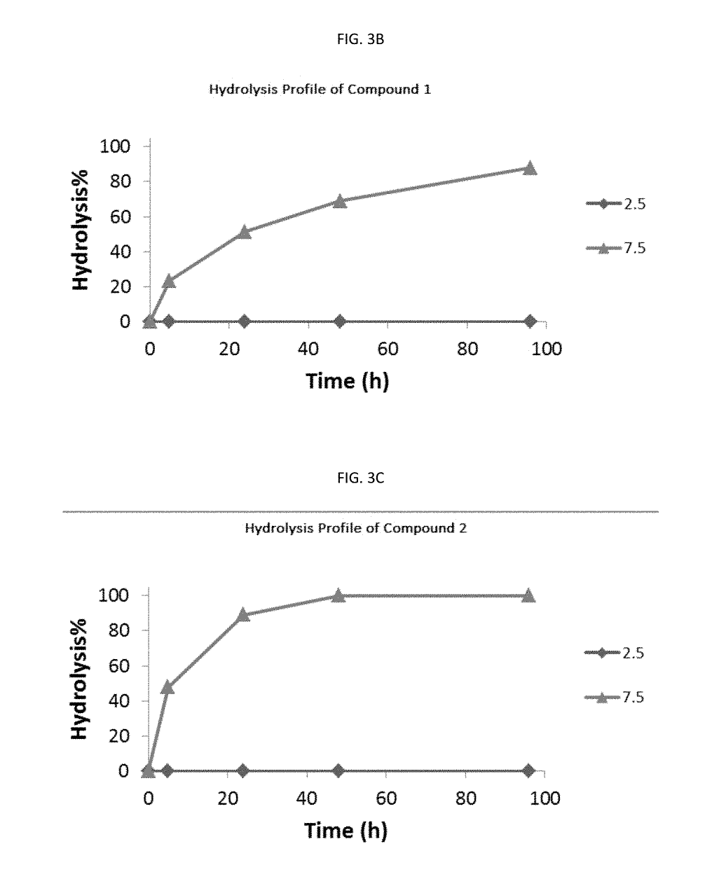

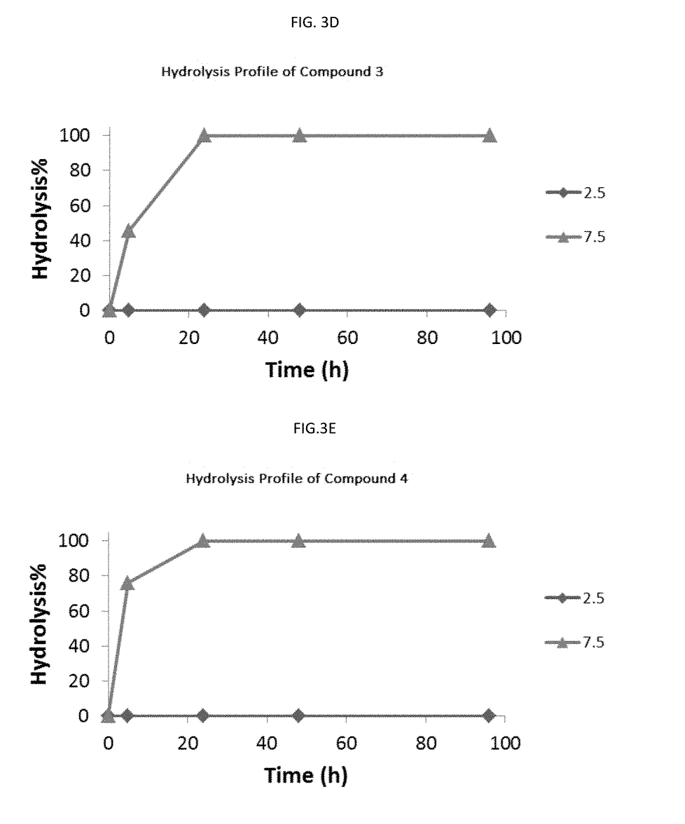

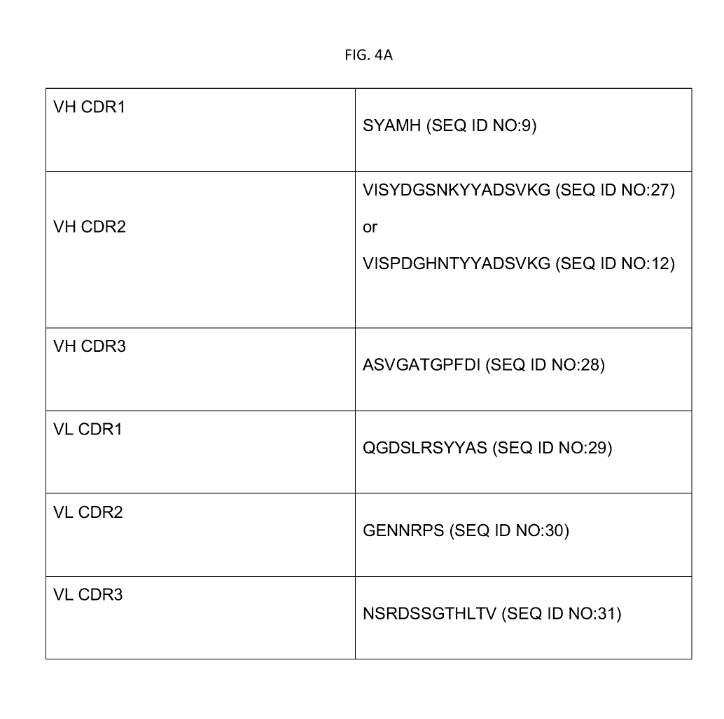

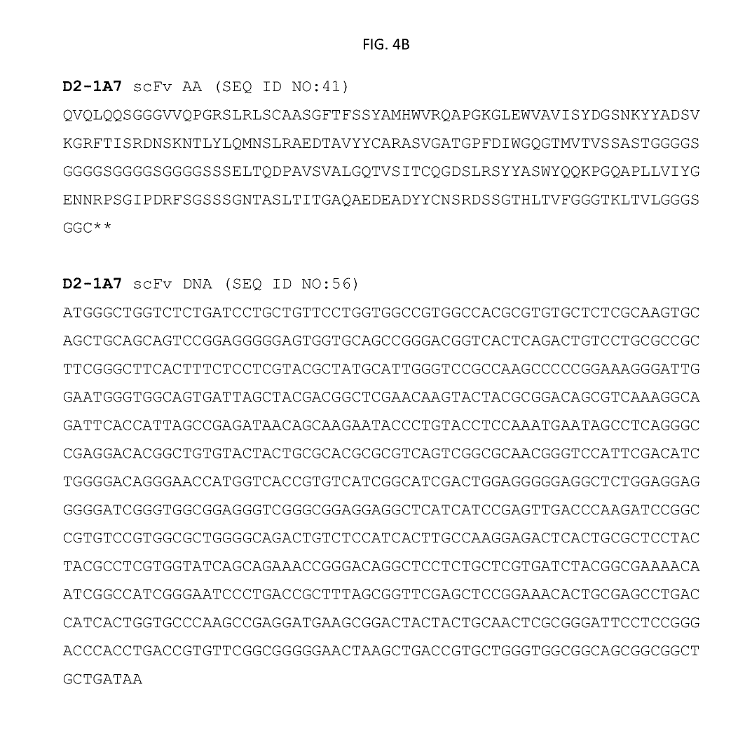

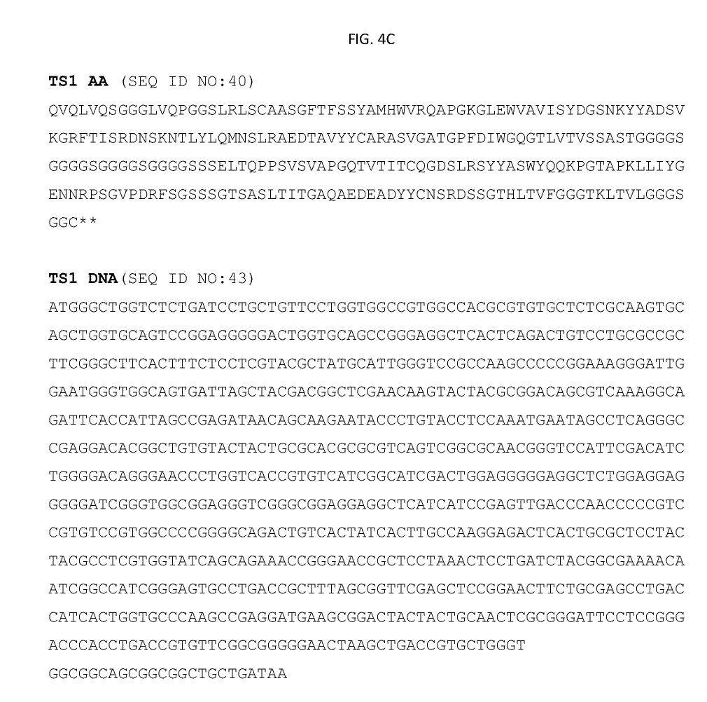

Preferred docetaxel prodrug compounds of formula (I) include compounds where (n) is 2 or 3, to provide a rapid hydrolysis rate at pH 7.5 and a sufficiently high relative hydrolysis rate for the compound at pH 7.5 compared to pH 2.5 (e.g., selecting docetaxel prodrugs with maximum hydrolysis rate of the docetaxel prodrug to docetaxel at pH 7.5 compared to the hydrolysis rate at pH 2.5). FIGS. 3C-3G show hydrolysis profiles for various examples of docetaxel prodrugs.

### EphA2 Targeted scFv Moiety

The docetaxel-generating liposome can comprise a EphA2 targeting moiety. The targeting moiety can be a single chain Fv (“scFv”), a protein that can be covalently bound to a liposome to target the docetaxel-producing liposomes disclosed herein. The scFv can be comprised of a single polypeptide chain in which a VH and a VL are covalently linked to each other, typically via a linker peptide that allows the formation of a functional antigen binding site comprised of VH and VL CDRs. An Ig light or heavy chain variable region is composed of a plurality of “framework” regions (FR) alternating with three hypervariable regions, also called “complementarity determining regions” or “CDRs”. The extent of the framework regions and CDRs can be defined based on homology to sequences found in public databases. See, for example, “Sequences of Proteins of Immunological Interest,” E. Kabat et al., Sequences of proteins of immunological interest, 4th ed. U.S. Dept. Health and Human Services, Public Health Services, Bethesda, Md. (1987). All scFv sequence numbering used herein is as defined by Kabat et al.

As used herein, unless otherwise indicated, the term “anti-EphA2 scFv” refers to an scFv that immunospecifically binds to EphA2, preferably the ECD of EphA2. An EphA2-specific scFv does not immunospecifically bind to antigens not present in EphA2 protein.

In certain embodiments, an scFv disclosed herein includes one or any combination of VH FR1, VH FR2, VH FR3, VL FR1, VL FR2, and VL FR3 set forth in Table 1. In one embodiment, the scFv contains all of the frameworks of Table 1 below.

TABLE 1
Exemplary Framework Sequences

| | |
|---|---|
| VH FR1 | QVQLVQSGGGLVQPGGSLRLSCAASGFTFS |
| (SEQ ID NO: 1) | |
| VH FR2 | WVRQAPGKGLEWVT |
| (SEQ ID NO: 2) | |
| VH FR3 | RFTISRDNSKNTLYLQMNSLRAEDTAVYYCAR |
| (SEQ ID NO: 3) | |
| VH FR4 | WGQGTLVTVSS |
| (SEQ ID NO: 4) | |
| VL FR1 | SSELTQPPSVSVAPGQTVTITC |
| (SEQ ID NO: 5) | |
| VL FR2 | WYQQKPGTAPKLLIY |
| (SEQ ID NO: 6) | |
| VL FR3 | GVPDRFSGSSSGTSASLTITGAQAEDEADYYC |
| (SEQ ID NO: 7) | |
| VL FR4 | FGGGTKLTVLG |
| (SEQ ID NO: 8) | |

In certain aspects, an scFv disclosed herein is thermostable, e.g., such that the scFv is well-suited for robust and scalable manufacturing. As used herein, a “thermostable” scFv is an scFv having a melting temperature (Tm) of at least about 70° C., e.g., as measured using differential scanning fluorimetry (DSF).

A preferred anti-EphA2 scFv binds to the extracellular domain of EphA2 polypeptide, i.e., the part of the EphA2 protein spanning at least amino acid residues 25 to 534 of the sequence set forth in GenBank Accession No. NP_004422.2 or UniProt Accession No. P29317.

In certain embodiments, an anti-EphA2 scFv disclosed herein includes a VH CDR1, VH CDR2, VH CDR3, VL CDR1, VL CDR2, and VL CDR3 each with a sequence as set forth in Table 2. Note that the VH CDR2 sequence (also referred to as CDRH2) will be any one selected from the 18 different VH CDR2 sequences set forth in Table 2.

TABLE 2
Complementary Determining Regions (CDRs)

| | |
|---|---|
| VH CDR1 (SEQ ID NO: 9) | SYAMH |
| VH CDR2 (SEQ ID NO: 10) | VISPAGNNTYYADSVK |
| VH CDR2 (SEQ ID NO: 11) | VISPAGRNKYYADSVK |
| VH CDR2 (SEQ ID NO: 12) | VISPDGHNTYYADSVKG |
| VH CDR2 (SEQ ID NO: 13) | VISPHGRNKYYADSVK |
| VH CDR2 (SEQ ID NO: 14) | VISRRGDNKYYADSVK |
| VH CDR2 (SEQ ID NO: 15) | VISNNGHNKYYADSVK |
| VH CDR2 (SEQ ID NO: 16) | VISPAGPNTYYADSVK |
| VH CDR2 (SEQ ID NO: 17) | VISPSGHNTYYADSVK |
| VH CDR2 (SEQ ID NO: 18) | VISPNGHNTYYADSVK |
| VH CDR2 (SEQ ID NO: 19) | AISPPGHNTYYADSVK |
| VH CDR2 (SEQ ID NO: 20) | VISPTGANTYYADSVK |
| VH CDR2 (SEQ ID NO: 21) | VISPHGSNKYYADSVK |
| VH CDR2 (SEQ ID NO: 22) | VISNNGHNTYYADSVK |
| VH CDR2 (SEQ ID NO: 23) | VISPAGTNTYYADSVK |
| VH CDR2 (SEQ ID NO: 24) | VISPPGHNTYYADSVK |
| VH CDR2 (SEQ ID NO: 25) | VISHDGTNTYYADSVK |
| VH CDR2 (SEQ ID NO: 26) | VISRHGNNKYYADSVK |
| VH CDR2 (SEQ ID NO: 27) | VISYDGSNKYYADSVKG |
| VH CDR3 (SEQ ID NO: 28) | ASVGATGPFDI |
| VL CDR1 (SEQ ID NO: 29) | QGDSLRSYYAS |
| VL CDR2 (SEQ ID NO: 30) | GENNRPS |
| VL CDR3 (SEQ ID NO: 31) | NSRDSSGTHLTV |

In certain embodiments, an scFv disclosed herein is an internalizing anti-EphA2 scFv. Binding of such an scFv to the ECD of and EphA2 molecule present on the surface of a living cell under appropriate conditions results in internalization of the scFv. Internalization results in the transport of an scFv contacted with the exterior of the cell membrane into the cell-membrane-bound interior of the cell. Internalizing scFvs find use, e.g., as vehicles for targeted delivery of drugs, toxins, enzymes, nanoparticles (e.g., liposomes), DNA, etc., e.g., for therapeutic applications.

Certain scFvs described herein are single chain Fv scFvs e.g., scFvs or (scFv′)2s. In such scFvs, the VH and VL polypeptides are joined to each other in either of two orientations (i.e., the VH N-terminal to the VL, or the VL N-terminal to the VH) either directly or via an amino acid linker. Such a linker may be, e.g., from 1 to 50, 5 to 40, 10 to 30, or 15 to 25 amino acids in length. In certain embodiments, 80% or greater, 85% or greater, 90% or greater, 95% or greater, or 100% of the residues of the amino acid linker are serine (S) and/or glycine (G). Suitable exemplary scFv linkers comprise or consist of the sequence:

(SEQ ID NO: 32)
ASTGGGGSGGGGSGGGGSGGGGS,
(SEQ ID NO: 33)
GGGGSGGGGSGGGGSGGGGS,
(SEQ ID NO: 34)
GGGGSGGGGSGGGGS,
(SEQ ID NO: 35)
ASTGGGGAGGGGAGGGGAGGGGA,
(SEQ ID NO: 36)
GGGGAGGGGAGGGGAGGGGA,
(SEQ ID NO: 37)
TPSHNSHQVPSAGGPTANSGTSGS,
and
(SEQ ID NO: 38)
GGSSRSSSSGGGGSGGGG.

An exemplary internalizing anti-EphA2 scFv is scFv TS1 (SEQ ID NO:40). In scFv TS1, and in certain other scFvs disclosed herein, the VH of the scFv is at the amino terminus of the scFv and is linked to the VL by a linker indicated in italics. The CDRs of the scFvs are underlined and are presented in the following order: VH CDR1, VH CDR2, VH CDR3, VL CDR1, VL CDR2, and VL CDR3.

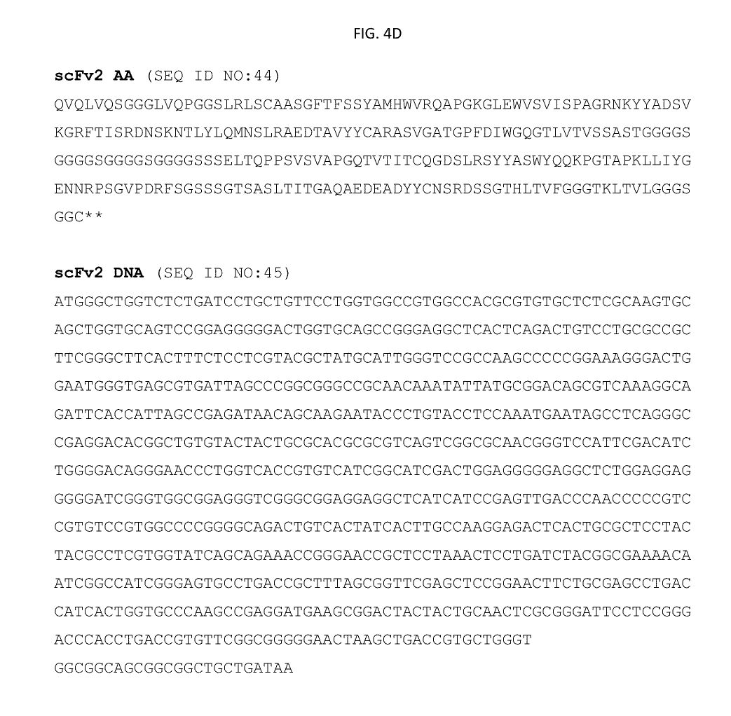

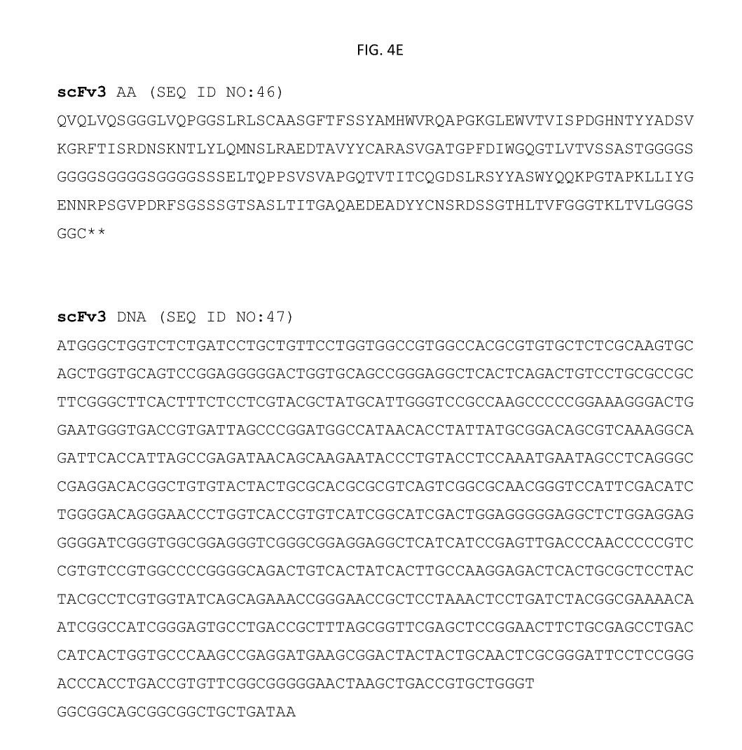

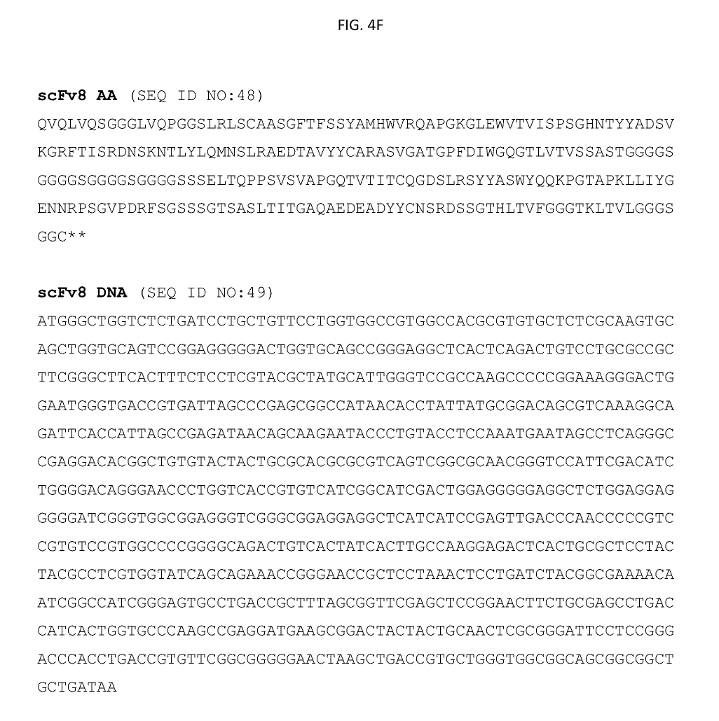

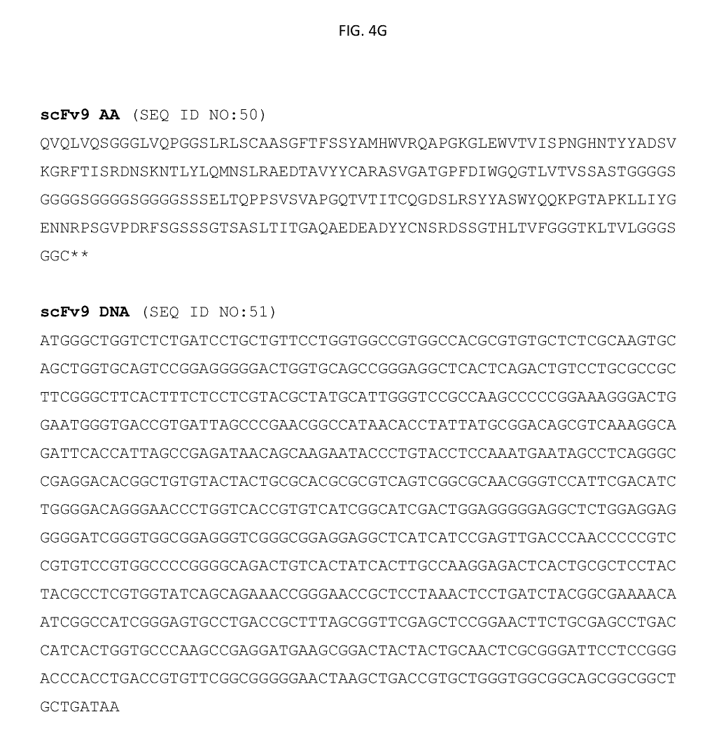

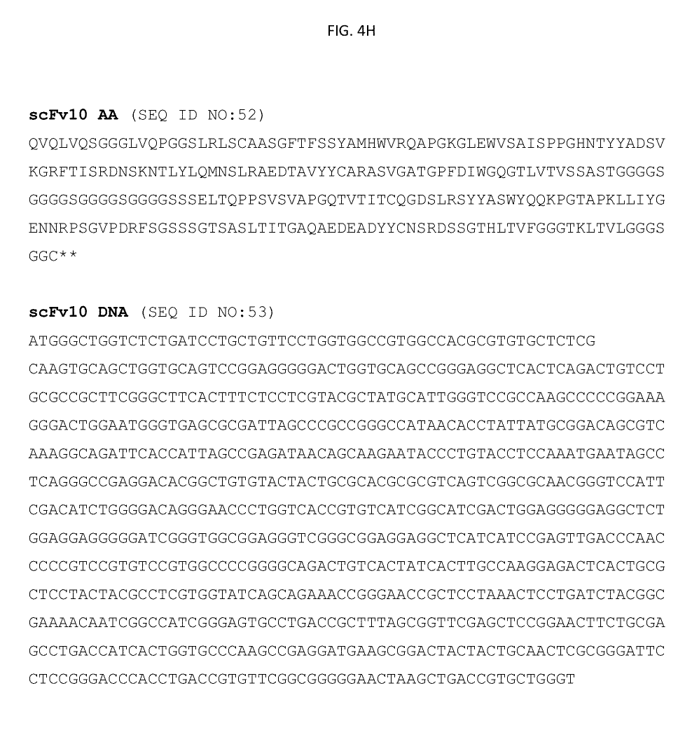

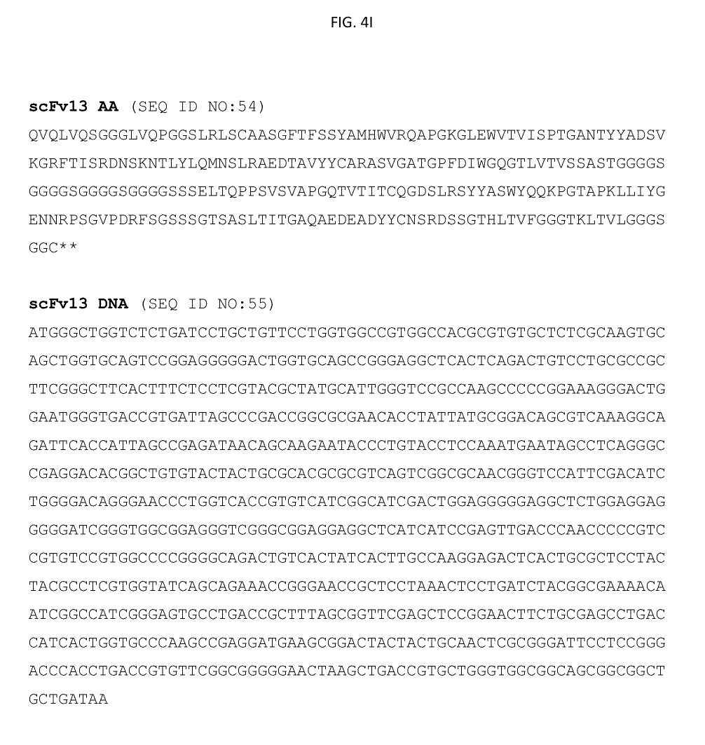

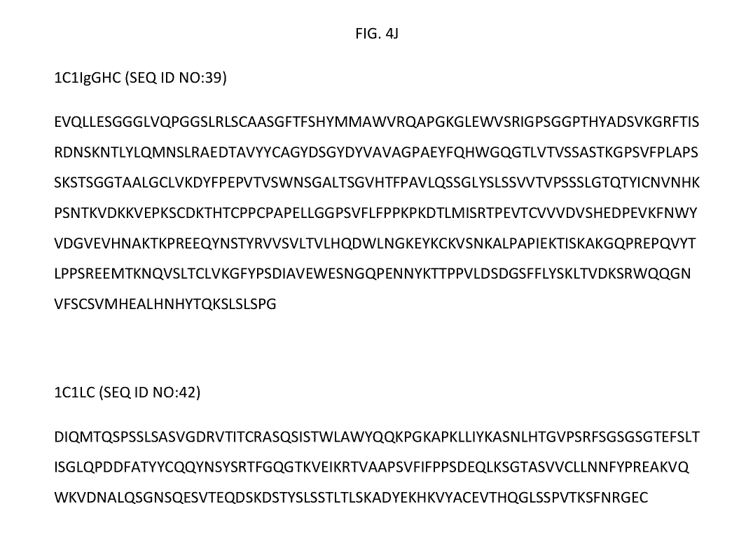

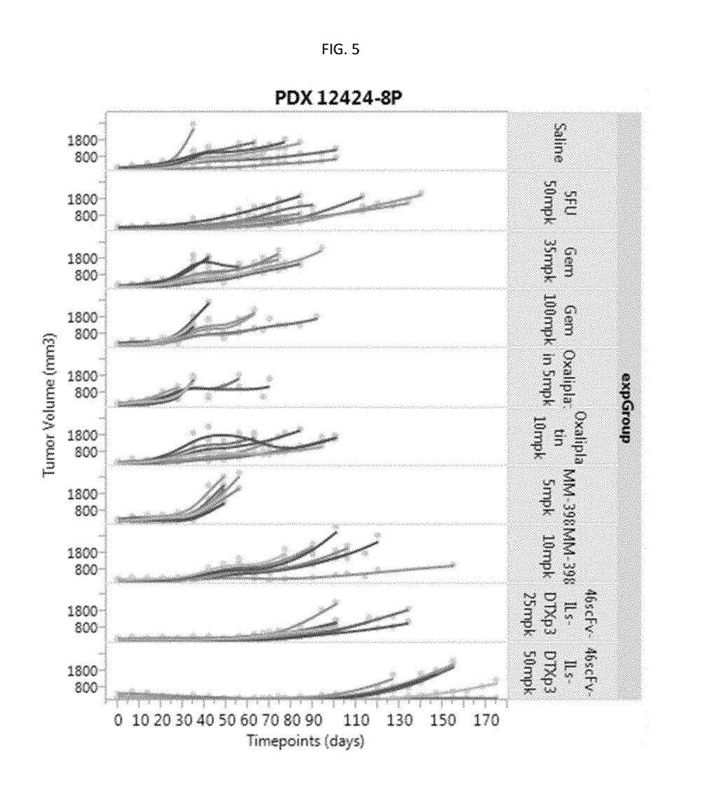

The docetaxel-generating EphA2-targeted liposomes can also include one or more EphA2 targeted scFv sequences shown FIG. 4B (SEQ ID NO:41, designated “D2-1A7”, encoded by the DNA sequence of SEQ ID NO:56 designated “D2-1A7 DNA”), or FIG. 4C (SEQ ID NO:40, designated “TS1”, encoded by the DNA sequence of SEQ ID NO:43 designated “TS1 DNA”), or FIG. 4D (SEQ ID NO:44, designated “scFv2”, encoded by the DNA sequence of SEQ ID NO:45 designated “scFv2 DNA”), or FIG. 4E (SEQ ID NO:46, designated “scFv3”, encoded by the DNA sequence of SEQ ID NO:47 designated “scFv3 DNA”), or FIG. 4F (SEQ ID NO:48, designated “scFv8”, encoded by the DNA sequence of SEQ ID NO:49 designated “scFv8 DNA”), or FIG. 4G (SEQ ID NO:50, designated “scFv9”, encoded by the DNA sequence of SEQ ID NO:51 designated “scFv9 DNA”) or FIG. 4H (SEQ ID NO:52, designated “scFv10”, encoded by the DNA sequence of SEQ ID NO:53 designated “scFv10 DNA”) or FIG. 4I (SEQ ID NO:54, designated “scFv13”, encoded by the DNA sequence of SEQ ID NO:55 designated “scFv13 DNA”).

Also provided are variants of scFv TS1 in which VH CDR2 is selected from any of the 18 different CDRH2 sequences set forth above in Table 2.

Using the information provided herein, the scFvs disclosed herein may be prepared using standard techniques. For example, the amino acid sequences provided herein can be used to determine appropriate nucleic acid sequences encoding the scFvs and the nucleic acids sequences then used to express one or more of the scFvs . The nucleic acid sequence(s) can be optimized to reflect particular codon “preferences” for various expression systems according to standard methods.

Using the sequence information provided herein, the nucleic acids may be synthesized according to a number of standard methods. Oligonucleotide synthesis, is conveniently carried out on commercially available solid phase oligonucleotide synthesis machines or manually synthesized using, for example, the solid phase phosphoramidite triester method. Once a nucleic acid encoding an scFv disclosed herein is synthesized, it can be amplified and/or cloned according to standard methods.

Expression of natural or synthetic nucleic acids encoding the scFvs disclosed herein can be achieved by operably linking a nucleic acid encoding the scFv to a promoter (which may be constitutive or inducible), and incorporating the construct into an expression vector to generate a recombinant expression vector. The vectors can be suitable for replication and integration in prokaryotes, eukaryotes, or both. Typical cloning vectors contain functionally appropriately oriented transcription and translation terminators, initiation sequences, and promoters useful for regulation of the expression of the nucleic acid encoding the scFv. The vectors optionally contain generic expression cassettes containing at least one independent terminator sequence, sequences permitting replication of the cassette in both eukaryotes and prokaryotes, e.g., as found in shuttle vectors, and selection markers for both prokaryotic and eukaryotic systems.

To obtain high levels of expression of a cloned nucleic acid it is common to construct expression plasmids which contain a strong promoter to direct transcription, a ribosome binding site for translational initiation, and a transcription/translation terminator, each in functional orientation to each other and to the protein-encoding sequence. The scFv gene(s) may also be subcloned into an expression vector that allows for the addition of a tag sequence, e.g., FLAG™ or His6, at the C-terminal end or the N-terminal end of the scFv (e.g. scFv) to facilitate identification, purification and manipulation. Once the nucleic acid encoding the scFv is isolated and cloned, one can express the nucleic acid in a variety of recombinantly engineered cells. Examples of such cells include bacteria, yeast, filamentous fungi, insect, and mammalian cells.

Isolation and purification of an scFv disclosed herein can be accomplished by isolation from a lysate of cells genetically modified to express the protein constitutively and/or upon induction, or from a synthetic reaction mixture, with purification, e.g., by affinity chromatography (e.g., using Protein A or Protein G). The isolated scFv can be further purified by dialysis and other methods normally employed in protein purification.

The present disclosure also provides cells that produce subject scFvs . For example, the present disclosure provides a recombinant host cell that is genetically modified with one or more nucleic acids comprising nucleotide sequence encoding an scFv disclosed herein. DNA is cloned into, e.g., a bacterial (e.g., bacteriophage), yeast (e.g. Saccharomyces or Pichia) insect (e.g., baculovirus) or mammalian expression system. One suitable technique uses a filamentous bacteriophage vector system. See,. e.g., U.S. Pat. Nos. 5,885,793; 5,969,108; and 6,512,097.

The EphA2 Targeted scFv Amino Acid Sequence can be attached to the liposome using an EphA2 (scFv) to maleimide-activated PEG-DSPE. For example, the scFv-PEG-DSPE drug substance can be a fully humanized single chain antibody fragment (scFv) conjugated to maleimide PEG-DSPE via the C-terminal cysteine residue of scFv. In some examples, the EphA2 targeted scFv is conjugated covalently through a stable thioether bond to a lipopolymer lipid, Mal-PEG-DSPE, which interacts to form a micellular structure. Preferably, the scFv is not glycosylated.

### Preparing EphA2-Targeted Liposomes for Delivery of Docetaxel

The docetaxel prodrug can be loaded into liposomes through different approaches. Remote loading methods enabls high loading efficiency and good scalability. Typically, liposomes are prepared in a loading aid (trapping agent) that may include a gradient-forming ion and a drug-precipitating or drug-complexing agent. The extraliposomal loading aid is removed, e.g., by diafiltration to generate an ion gradient across the liposome bilayer. Selected drug can cross the lipid bilayer, accumulate inside the liposome at the expense of the ion gradient and form complexes or precipitates with the loading aid. If the liposome lipid is in the gel state at ambient temperature, the loading is effected at elevated temperatures where the liposome membrahe is in the liquid crystalline state. When drug loading is complete, liposomes are rapidly chilled so that loaded drug can be retained by the rigid membrane. Any factor involved in the drug loading step may impact the loading efficiency.

The EphA2 targeted nano-liposome can be obtained by combining the Eph-A2 binding scFv with DSPE-PEG-Mal under conditions effective to conjugate the scFv to the DSPE-PEG-Mal moiety. The DSPE-PEG-Mal conjugate can be combined with a polysulfated polyol loading aid and other lipid components to form a liposome containing the polysulfated polyol encapsulated with a lipid vesicle.

Referring again to FIG. 1B, the drug can be loaded into a liposome encapsulating a trapping agent. The drug release rate can be controlled by varying the type and concentration of the trapping agents, as can the stability towards hydrolysis of the prodrug. Examples of trapping agents include but are not limited to ammonium sucroseoctasulfate (SOS), diethylammonium SOS (DEA-SOS), triethylammonium SOS (TEA-SOS), and diethylammonium dextran sulfate. The concentration of the trapping agent can be selected to provide desired drug loading properties, and can vary from 250 mN to 2 N depending on the drug to lipid ratio desired. Normality (N) of the trapping agent solution depends on the valency of its drug-complexing counter-ion and is a product of the counter-ion molarity and its valency. For example, the normality of DEA-SOS solution, SOS being an octavalent ion, is equal to SOS molar concentration times eight. Thus, 1 N SOS is equal to 0.125 M SOS. When DEA-SOS is used as the trapping agent, the concentration ranges preferably from 0.5 N to 1.5 N, most preferably from 0.85 N to 1.2 N A formulation employing TEA-SOS at 1.1 N can result in a final formulation containing 300-800 grams of docetaxel equivalent prodrug per mol of phospholipid. This results in a dose of lipid that is between 8 and 22 mg total lipid/kg (302-806 mg/m²) to patients at a dose of 250 mg docetaxel equivalents/m². The final formulation has a preferable drug-to-phospholipid ratio of 250-400 g docetaxel equivalents/mol phospholipid.

Docetaxel prodrugs can be dissolved in either acidic buffer directly, or in the presence of other solubilizing reagents such as hexa(ethylene glycol) (PEG6) or poly(ethylene glycol) 400 (PEG-400). Under any circumstance, basic conditions should be avoided in the solubilization process for docetaxel prodrugs that hydrolyze under basic conditions.

Liposomes used for loading taxane prodrugs are prepared by ethanol extrusion methods. The lipid components can be selected to provide desired properties.

In general, a variety of lipid components can be used to make the liposomes. Lipid components usually include, but are not limited to (1) uncharged lipid components, e.g., cholesterol, ceramide, diacylglycerol, acylpoly(ethers) or alkylpoly(ethers) and (2) neutral phospholipids, e.g., diacylphosphatidylcholines, dialkylphosphatidylcholines, sphingomyelins, and diacylphosphatidylethanolamines. Various lipid components can be selected to fulfill, modify or impart one or more desired functions. For example, phospholipid can be used as principal vesicle-forming lipid. Inclusion of cholesterol is useful for maintaining membrane rigidity and decreasing drug leakage. Polymer-conjugated lipids can be used in the liposomal formulation to increase the lifetime of circulation via reducing liposome clearance by liver and spleen, or to improve the stability of liposomes against aggregation during storage, in the absence of circulation extending effect.

Preferably, the liposome comprises an uncharged lipid component, a neutral phospholipid component and a polyethylene (PEG)-lipid component. A preferred PEGylated lipid component is PEG(Mol. weight 2,000)-distearoylglycerol (PEG-DSG) or N-palmitoyl-sphingosine-1-{succinyl[methoxy(polyethylene glycol)2000]} (PEG-ceramide). For example, the lipid components can include egg sphingomyelin, cholesterol, PEG-DSG at a suitable molar ratio (e.g., comprising sphingomyelin and cholesterol at a 3:2 molar ratio with a desired amount of PEG-DSG). The amount of PEG-DSG is preferably incorporated in the amount of 10 mol % (e.g., 4-10 mol %) of the total liposome phospholipid, or less, such as, less than 8 mol % of the total phospholipid, and preferably between 5-7 mol % of the total phospholipid. In another embodiment, a sphingomyelin (SM) liposome is employed in the formulation which is comprised of sphingomyelin, cholesterol, and PEG-DSG-E at given mole ratio such as 3:2:0.03. The neutral phospholipid and PEG-lipid components used in this formulation are generally more stable and resistant to acid hydrolysis. Sphingomyelin and dialkylphosphatidylcholine are examples of preferred phospholipid components. More specifically, phospholipids with a phase transition temperature (Tₘ) greater than 37° C. are preferred. These include, but are not limited to, egg-derived sphingomyelin, 1,2-di-O-octadecyl-sn-glycero-3-phosphocholine, N-stearoyl-D-erythro-sphingosylphosphorylcholine, and N-palmitoyl-D-erythro-sphingosylphosphorylcholine. The choice of liposome formulation depends on the stability of specific prodrug under certain conditions and the cost of manufacturing.

Taxane prodrugs are loaded into liposomes at acidic pH ranging preferably from 4 to 6 in the presence of buffers preferably 5-40 mM. Suitable acidic buffers include but not limited to, 2-(N-morpholino)ethanesulfonic acid (MES), oxalic acid, succinic acid, manolic acid, glutaric acid, fumaric acid, citric acid, isocitric acid, aconitic acid, and propane-1,2,3-tricarboxylic acid. Different drug loading methods have been developed to facilitate efficient loading of taxane prodrugs into liposome. In one embodiment, prodrug solution is mixed with the liposome at room temperature first, followed by the pH adjustment and incubation at elevated temperature. In another embodiment, the pH of the prodrug solution and liposomes are adjusted first to desired loading pH, pre-warmed to the desired loading temperature, then mixed and incubated. In still another embodiment, prodrug is solubilized in 80% PEG6 solution at high concentration first, and added portion by portion into the pre-warmed liposome. In further embodiments, prodrugs are dissolved in 80% PEG400 first, diluted to about 8% PEG400 in dextrose MES buffer, mixed with liposome at room temperature first, then warmed up to the loading temperature.

Unencapsulated polysulfated polyol material can be removed from the composition. Then, the liposome containing the polysulfated polyol loading aid (preferably TEA-SOS or DEA SOS) can be contacted with the a suitable taxane or taxane prodrug, such as a docetaxel prodrug of Formula (I), preferably a docetaxel prodrug of Compound 3, Compound 4 or Compound 6, under conditions effective to load taxane or taxane prodrug into the liposome, preferably forming a stable salt with the encapsulated polysulfated polyol within the liposome. Simultaneously, the loading aid counter ion (e.g., TEA or DEA) leaves the liposome as the drug is loaded into the liposome. Finally, unencapsulated drug (e.g., docetaxel prodrug) is removed from the composition comprising the liposome. Methods of liposome drug loading are described in U.S. Pat. No. 8,147,867, filed May 2, 2005, and incorporated by reference.

Examples of methods suitable for making liposome compositions include extrusion, reverse phase evaporation, sonication, solvent (e.g., ethanol) injection, microfluidization, detergent dialysis, ether injection, and dehydration/rehydration. The size of liposomes can be controlled by controlling the pore size of membranes used for low pressure extrusions or the pressure and number of passes utilized in microfluidization or any other suitable methods. In one embodiment, the desired lipids are first hydrated by thin-film hydration or by ethanol injection and subsequently sized by extrusion through membranes of a defined pore size; most commonly 0.05 μm, 0.08 μm, or 0.1 μm. Preferably, the liposomes have an average diameter of about 90-120 nm, more preferably about 110 nm.

### EXAMPLES

Unless otherwise indicated, an exemplary EphA2 targeted docetaxel-generating nanoliposome composition designated “EphA2-Ls-DTX′” was tested as described in the examples below. EphA2-Ls-DTX' is a targeted liposome comprising a compound of Formula (I) designated Compound 3 encapsulated in a lipid vesicle formed from egg sphingomyelin, cholesterol and PEG-DSG in a weight ratio of about 4.4:1.6:1. The lipid vesicle also includes a scFv moiety of SEQ ID NO:46 covalently bound to PEG-DSPE in a weight ratio of about 1:32 of the total amount of PEG-DSPE in the lipid vesicle. The EphA2-Ls-DTX′ liposome can be formulated in a suitable composition to form a drug product, including a buffer system (e.g., citric acid and sodium citrate), an isotonicity agent (e.g., sodium chloride) and a sterile water vehicle as a diluent (e.g., water for injection).

In Examples 1-3, the anti-tumor efficacy of 46scFv-ILs-DTXp3 was compared to several standard of care agents, including the current front line treatment of choice of nab-Paclitaxel+Gemcitabine, in patient derived xenograft (PDX) models of pancreatic cancer. Primary tumor xenografts, serially maintained as explants, are capable of simulating the heterogeneity and genetic diversity observed in the patient population. Most importantly, these xenografts tend to preserve both the tissue architecture as well as drug sensitivity profiles initially seen in the donor primary tumor. As such, they likely represent a more clinically relevant model than traditional cell line implanted xenografts. The pancreatic xenograft model series are true xenotransplant models that were directly engrafted from patient tumor resections into SCID mice for propagation and maintained by transplantation of tumor fragments (Hylander et al., 2005, 2013). Experiments were performed according to approved guidelines. CB.17 SCID mice were obtained from Roswell Park Cancer Institute, initially at 6-8 weeks of age. Per treatment group, 8 animals were treated, unless otherwise indicated. Tumor pieces were derived from donor mice and engrafted subcutaneously. Depending on the variability in tumor growth, animals were either randomized to the different arms at one specific timepoint or a rolling randomization was performed in which a subgroup of animals were randomized through a period of time to ensure less variability in starting sizes. Animals were randomized and dosing initiated when tumors reached an average volume of 200-250 mm³ (range 100-400 mm³), unless otherwise indicated.

For efficacy experiments, 46scFv-ILs-DTXp3 were generated as described in composition description. All standard of care agents were purchased from curascript (Lake Mary, Fla.). MM-398 was generated in house using the final commercial process.

Intravenous administration of the indicated doses of each agent was initiated when tumors reached an average volume of 200-250 mm³ and continued for a total of four weekly doses. Tumor volumes were measured once to twice weekly during the dosing cycle and until tumors regrow or reaching maximum monitoring period of 120-160 days or animals were in poor general health and needed to be sacrificed. The tumor progression was monitored by palpation and caliper measurements of the tumors along the largest (length) and smallest (width) axis twice a week. The tumor sizes were determined twice weekly from the caliper measurements using the formula (Geran, R. I., et al., 1972 Cancer Chemother. Rep. 3:1-88):

Tumor volume (TV)=[(length)×(width)²]/2

Single tumor volume curves for each animal were plotted and two metrics that describe antitumor effects were calculated as follows:

1) Max Response=[(minimum TV−TV at day 0)/TV at day 0]×100

2) Time to regrowth=Time for the tumor to reach four times its initial size

All statistical analysis between treatment groups was performed using JMP v11.0 software. For treatment group comparison, two-way ANOVA analysis was performed in conjunction with post hoc Tukey HSD statistical analysis.

### Example 1: Efficacy of 46scFv-ILs-DTXp3 Versus Standard of Care Therapy in Pancreatic Patient Derived Xenografts

### Example 1A: The #12424 PDX Tumor Model

The #12424 PDX tumor model was described in Hylander (2005). The tumor material was collected from a 64 year old Caucasian male, who had been a life-long non-smoker. The cancer histological subtype was C25.7 (ICD-O-3 histology code 85033). The tumor was characterized as poorly differentiated, infiltrating ductal carcinoma, not otherwise specified with staging pT3, pN1 and M0. Histological staging per American Joint Committee on Cancer (5^{th} edition) was 2B. No follow-up treatment is available. The xenograft model was resistant to APO2L/Trail and to Gemcitabine treatment. The model had elevated levels of FGFR2 mRNA and was sensitive to Dovitinib (40 mg/kg) (Zhang et al., 2013). Model #12424 was maintained by passaging tumor fragments in immunodeficient mice. This PDX model was at passage 8 for study #12424-8P. FIG. 5 is a graph showing tumor growth curves for PDX 12424-8P.

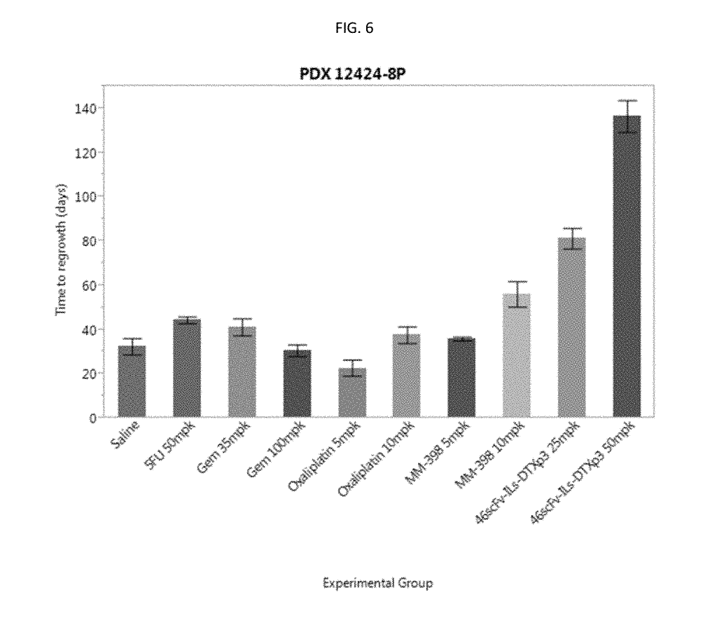

Results: Tumor growth profiles for tumors treated with several commonly used standard of care agents (5-Fluorouracil, Gemcitabine, Oxaliplatin) suggest moderate inhibition of tumor growth when compared to control (FIG. 5). A single dose level treatment of 50 mg/kg of 5-Fluorouracil (5 FU) at weekly doses for 4 weeks showed a minor, but not statistically significant, inhibition of tumor growth compared to saline control of 12 days (p=0.6965), as determined via measuring time to regrowth; defined as the time it takes for the tumor to reach 4x the tumor volume at time of treatment initiation. In a similar fashion, growth inhibition of the PDX 12424-8P model at both dose levels of Gemcitabine (35 mg/kg and 100 mg/kg) and Oxaliplatin (5 mg/kg and 10 mg/kg) monotherapy were statistically insignificant from saline control in terms of growth inhibition. MM-398, known commercially as Onivyde, demonstrated a 24 day (p=0.0065) inhibition of growth at the 10 mg/kg dose level; roughly double that of the most effective “traditional” chemotherapy 5-FU. However, 5 mg/kg of MM-398 did not show a significant advantage in tumor growth inhibition over non-liposomal standard of care agents. Overall the greatest inhibition of tumor growth belonged to the 46scFv-ILs-DTXp3 cohort, with 25 mg/kg showing a 49 day (p<0.0001) advantage in tumor growth inhibition compared to control and 50 mg/kg inhibiting growth for 104 days (p<0.0001)(FIG. 6).

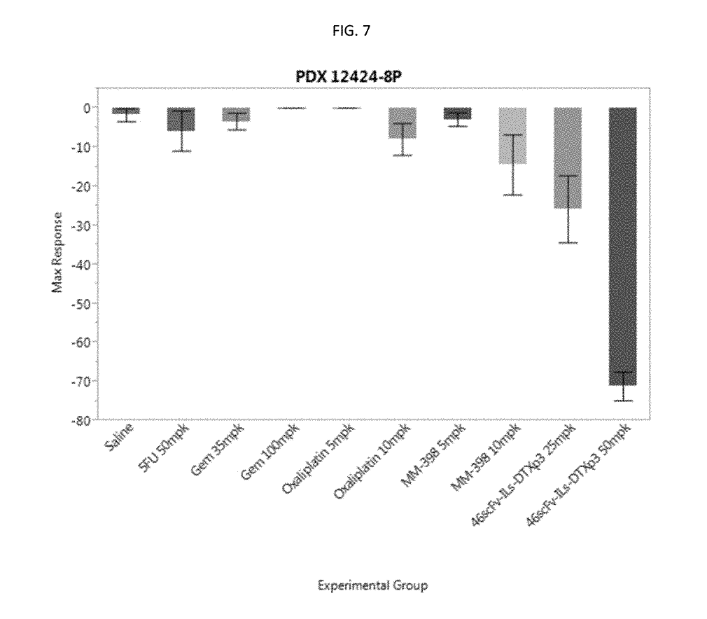

Using maximal response as a metric, we see similar trend in terms of efficacy in this model. Standard chemotherapy, as well as both the 5 mg/kg and 25 mg/kg dose of MM-398 did not generate a statistically significant response when compared to control. Overall, the largest response to drug was observed in the 46scFv-ILs-DTXp3 treatment groups, with 25 mg/kg showing a 24.16% (p=0.0084) increase in response to drug, while the 50 mg/kg groups showed a 69.5% (p<0.0001) increase compared to control (FIG. 7). At 50 mpk, 46scFv-ILs-DTXp3 had a more potent anti-tumor activity than all the other tested compounds which is measurable by maximum response and/or time to regrowth.

FIG. 6 is a graph showing the time to regrowth for PDX 12424-8P. FIG. 7 is a graph showing the maximum response for PDX 12424-8P.

### Example 1E3 PDX Model #14244

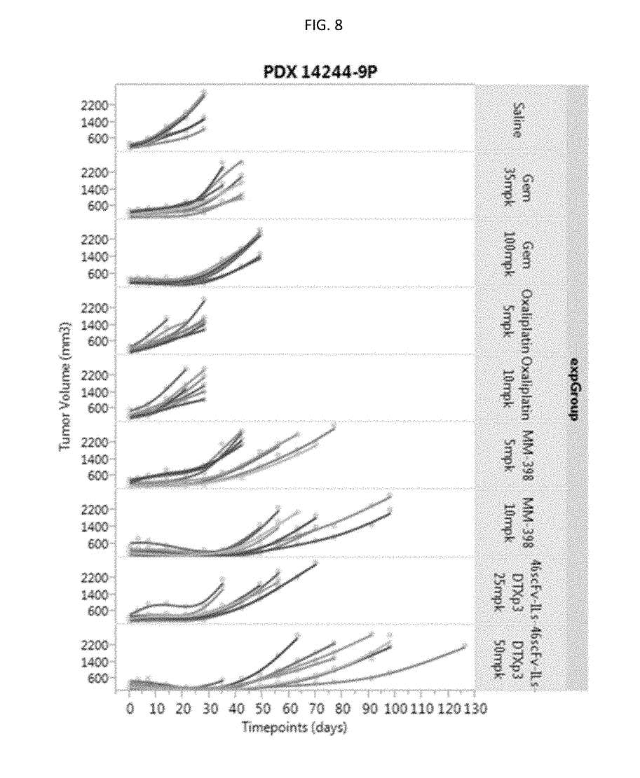

PDX model #14244 originated in the ampulla of Vater, also known as the hepatopancreatic duct, and is considered a relevant pancreatic model due to histology representative of pancreatic cancer (Sharma et al., 2014). This model has been shown to have elevated levels of FGFR2 mRNA (Zhang et al., 2013) and was sensitive to Apo2L/TRAIL treatment (Sharma et al., 2014). Growth from implantation occurred within 39 days and liver metastasis were found at 21 weeks. Model #14244 was maintained by passaging tumor fragments in immunodeficient mice. This PDX model was at passage 9 for study #14244-9P. FIG. 8 is a graph showing tumor growth for PDX 14244-9P.

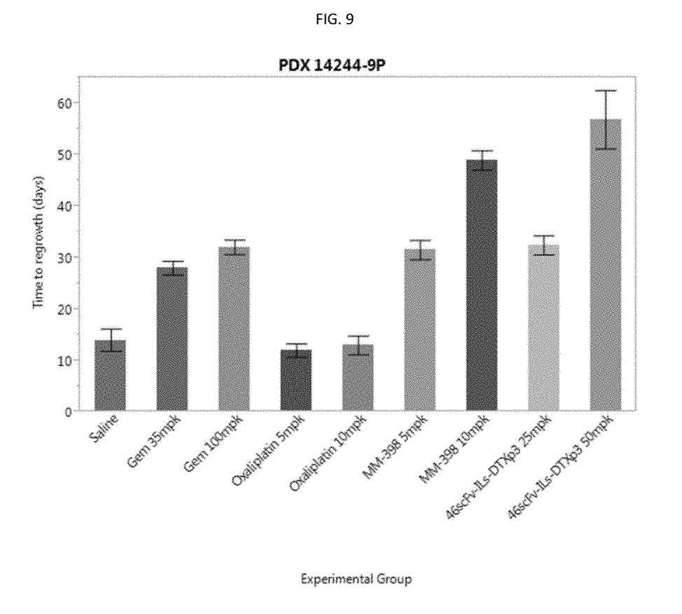

Results: In model PDX 14244-9P, both Oxaliplatin dose levels (5 and 10 mg/kg) did not confer a significant survival advantage over the control group (2 days, p=0.9998 and 1 day, P=1.0 respectively)(FIG. 8). Gemcitabine, however, did show a survival advantage compared to saline, with 35 mg/kg delaying regrowth by two weeks (p=0.0095) while the 100 mg/kg dose extended regrowth for 18 days (p=0.0004). The liposomal drug cohort, at the lower dose levels, showed similar tumor growth inhibition with 5 mg/kg MM-398 delaying regrowth of the tumors by 17.5 days (p=0.0004) and 35 mg/kg 46scFv-ILs-DTXp3 for 18.38 days (p=0.00002). The greatest tumor inhibition in this study was evidenced by the 50 mg/kg 46scFv-ILs-DTXp3 cohort (42.88, p<0.0001), with 10 mg/kg MM-398 following with a close second (35 days, p<0.0001) (FIG. 9).

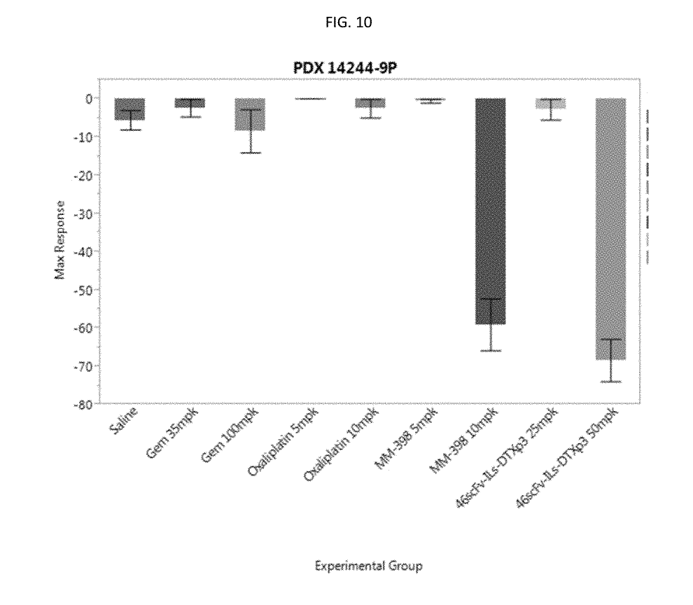

Looking at a secondary metric of efficacy, maximal tumor response to drug, we observed that both dose levels of Oxaliplatin and Gemcitabine, as well as the two lowest dose levels of MM-398 and 46scFv-ILs-DTXp3, did not demonstrate a significant tumor response to drug when compared to the control group. Conversely, 10 mg/kg MM-398 and 50 mg/kg 46scFv-ILs-DTXp3 both showed strong tumor response to drug with MM-398 providing a 53.54% (p<0.0001) tumor volume decrease and 46scFv-ILs-DTXp3 a 62.9% (p<0.0001) decrease (FIG. 10). At 50 mpk, 46scFv-ILs-DTXp3 had a more potent anti-tumor activity than almost all the other tested compounds, except for the 10 mg/kg dose of MM-398, which is measurable by maximum response and/or time to regrowth

FIG. 9 is a graph showing the time to regrowth for PDX 14244-9P. FIG. 10 is a graph showing the maximum response for PDX 14244-9P.

### Example 1C Pancreatic PDX Model #15010

Pancreatic PDX model #15010 tumor tissue was collected from a 74 year old Caucasian female. The tumor was located in the head of the pancreas (ICD-O-3 histology code 85033). The tumor was characterized as poorly differentiated, infiltrating ductal carcinoma, not otherwise specified with staging pT3, pN1 and M0. Histological staging per American Joint Committee on Cancer (6^{th} edition) was 2B (Hylander et al., 2013). The patient did not receive further therapy. Model #15010 was maintained by passaging tumor fragments in immunodeficient mice. At the time of implantation for the current study, this PDX model was at passage 5.

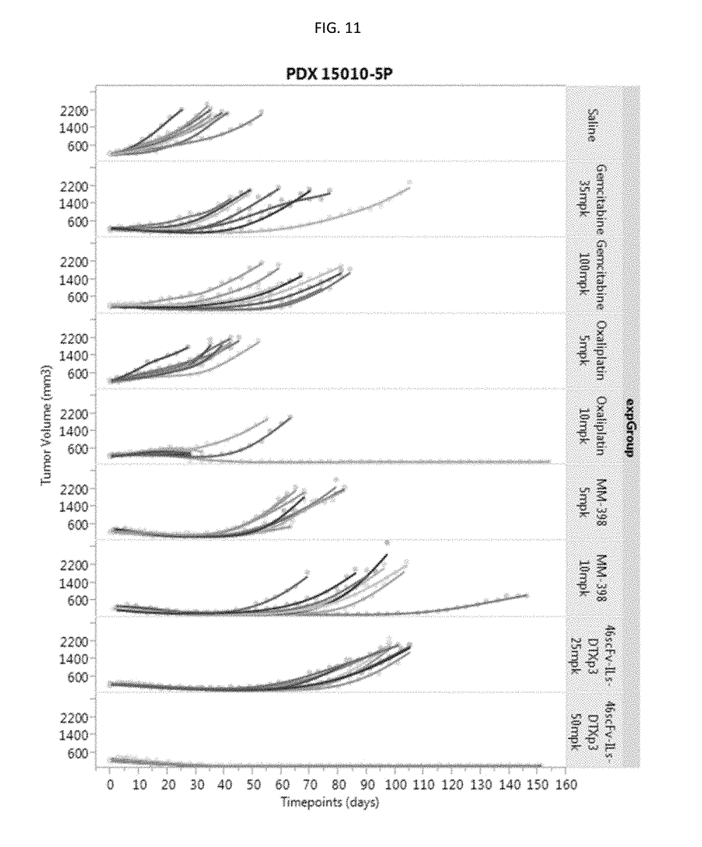

FIG. 11 is a graph showing tumor growth curves for PDX 15010-P5.

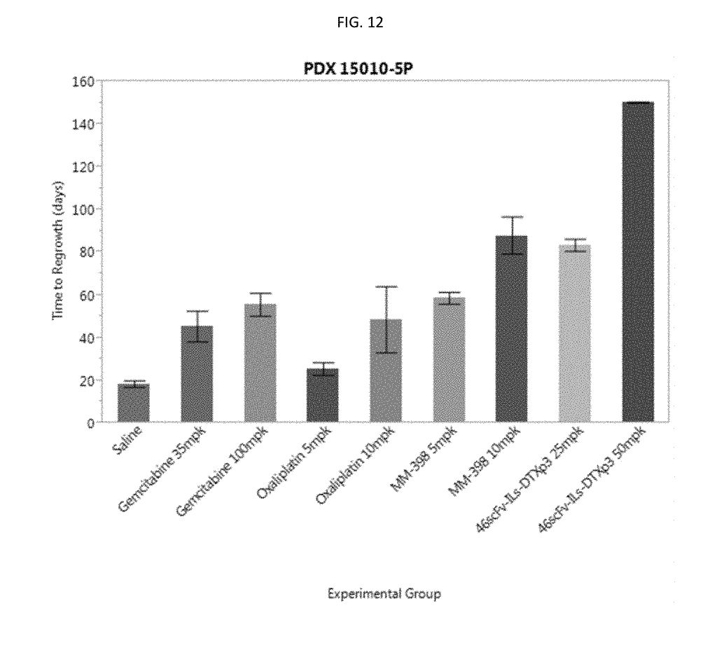

Results: This model showed moderate tumor inhibition, compared to saline control group, for all drugs tested with the exception of 5 mg/kg Oxaliplatin) (FIG. 11). While the other dose level of Oxaliplatin, 10 mg/kg, demonstrated activity, inhibiting tumor growth for 30 days, it narrowly missed statistical significance, with a p-value of 0.0631. The other non-liposomal chemotherapeutic tested, Gemcitabine, did inhibit tumor growth relative to control at both dose levels (35 mg/kg=27 days; 100 mg/kg=37 days), only the 100 mg/kg group reached significance with a p-value of 0.0080. Regarding the liposomal groups, MM-398 at 5 mg/kg demonstrated tumor growth inhibition (40 days, p=0.0030) similar to the non-liposomal drugs. As one would expect, the higher 10 mg/kg dose level of MM-398 improved on the 5 mg/kg finding, with a prolonging of tumor regrowth by 70 days (p<0.0001). Interestingly, the 25 mg/kg 46scFv-ILs-DTXp3 treatment showed relatively similar activity to 10 mg/kg MM-398 dose level, prolonging time to regrowth by 65 days (p<0.0001) compared to MM-398's 70 days. By far, however, the longest time to regrowth conferred by drugs tested belonged to the 50 mg/kg 46scFv-ILs-DTXp3 cohort (132 days, p<0.0001), roughly doubling the time to regrowth conferred by 10 mg/kg MM-398 and 35 mg/kg 46scFv-ILs-DTXp3 (FIG. 12).

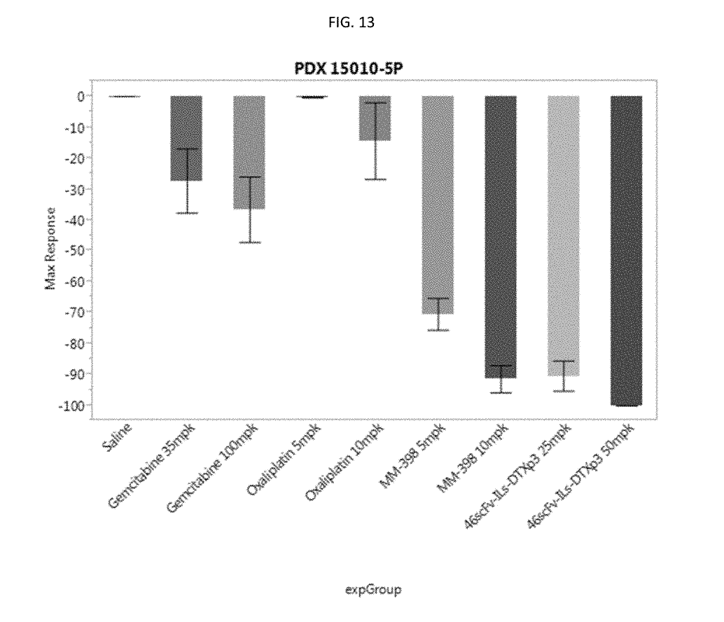

Regarding maximal tumor response to drug, both Oxaliplatin treatment doses did not show a statistically significant difference from saline control (FIG. 13). While the Gemcitabine groups did show hints of activity (35 mg/kg=27%, 100 mg/kg=37% decrease in tumor volume), only the 100 mg/kg group met statistical significance with at p=value of 0.0105. Again, in terms of tumor response, the liposomal formulations proved superior to traditional chemotherapeutics. The 5 mg/kg MM-398 treatment showed a maximal decrease in tumor volume of 70% (p<0.0001) while the 10 mg/kg MM-398 dose improved on that by 21% (91% compared to saline control, p<0.0001). In this model, both dose levels of 46scFv-ILs-DTXp3 demonstrated roughly similar activity with 25 mg/kg 46scFv-ILs-DTXp3 yielding a 90% max decrease in tumor volume while the 50 mg/kg 46scFv-ILs-DTXp3 (p<0.0001) treatment group decreased tumor volume by 100% (p<0.0001). At 50 mg/kg, 46scFv-ILs-DTXp3 had a more potent anti-tumor activity than all the other tested compounds which is measurable by time to regrowth. In terms of max response, 50 mg/kg, 46scFv-ILs-DTXp3 was statistically indistinguishable from 25 mg/kg, 46scFv-ILs-DTXp3 (9.6% differential, p=0.9859) and the 10 mg/kg dose of MM-398 (8.6% differential, p=0.9930)

FIG. 12 is a graph showing the time to regrowth for PDX 15010-P5. FIG. 13 is a graph showing the maximum response for PDX 15010-P5.

### Example 2 46scFv-ILs-DTXp3 vs. Abraxane

This study tested five distinct pancreatic PDX models for their response to differing levels of both nab-Paclitaxel and 46scFv-ILs-DTXp3. In all models, treatment with chemotherapy inhibited tumor growth. As expected, inhibition of tumor growth was dose dependent in the drug treatment group, with the largest inhibition of tumor regrowth in all models found in the highest 46scFv-ILs-DTXp3 dosage, 50 mg/kg.

In terms of maximal tumor response to drug, 46scFv-ILs-DTXp3 at 50 mg/kg demonstrated superiority in all models tested when compared to nab-Paclitaxel at 30 mg/kg dose level. 46scFv-ILs-DTXp3 showed superior anti-tumor effect measured by maximum response and/or time to regrowth. This was true in most tested models when comparing 46scFv-ILs-DTXp3 at 50mpk vs nab-Paclitaxel at 30 mpk or 46scFv-ILs-DTXp3 at 25 mpk vs nab-Paclitaxel at 15 mpk.

### Example 2A 14312 PDX Tumor Model

The #14312 PDX tumor material was collected from a 64 year old Caucasian male, who had been a reformed smoker for >15 years. The tumor was located in the head of the pancreas (ICD-O-3 histology code 85033). The tumor was characterized as infiltrating ductal carcinoma with staging pT3 pN1a MX. Histological staging per American Joint Committee on Cancer (6th edition) was 2B. The patient progressed after receiving Gemcitabine for approximately 2 months after the initial surgery. PDX model #14312 was evaluated by Zhang (2013) and found to have elevated levels of FGFR2 mRNA. Model #14312 was maintained by passaging tumor fragments in immunodeficient mice. At the time of implantation for the current study, this PDX model was at passage 4.

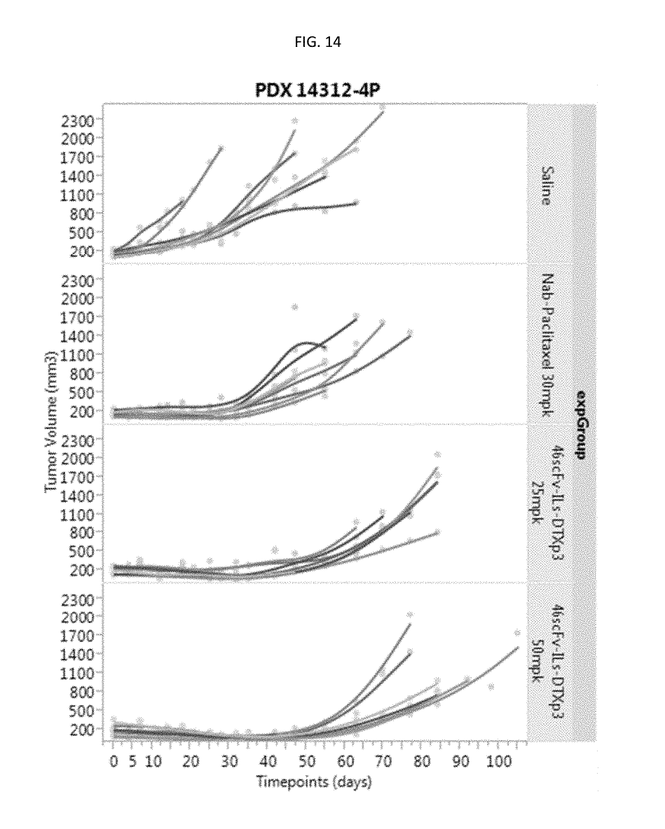

FIG. 14 is a graph showing tumor growth curves for PDX-14312-4P.

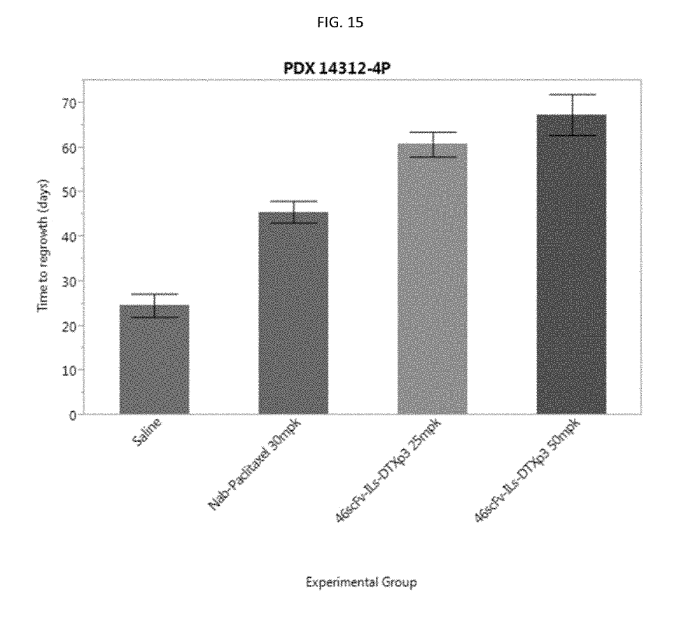

Tumors treated with 30 mg/kg nab-Paclitaxel (n=8) had a mean tumor volume of 165±15 mm³ at treatment initiation. Tumors treated with 30 ng/kg nab-Paclitaxel increased steadily in size with a moderate increase in the time to regrowth (21 days compared to saline control; p=0.0004), defined as the time it takes for the tumor to reach a volume of four times initial tumor volume. In this model, both doses of 46scFv-ILs-DTXp3, 25 mg/kg and 50 mg/kg, demonstrated similar efficacy at inhibiting tumor regrowth at 61±3 and 67±5 days respectively. Compared with 30 mg/kg nab-Paclitaxel, both 46scFv-ILs-DTXp3 doses achieved statistically significant inhibition of tumor regrowth (25 mg/kg p=0.0127; 50 mg/kg p=0.0003) (FIG. 15).

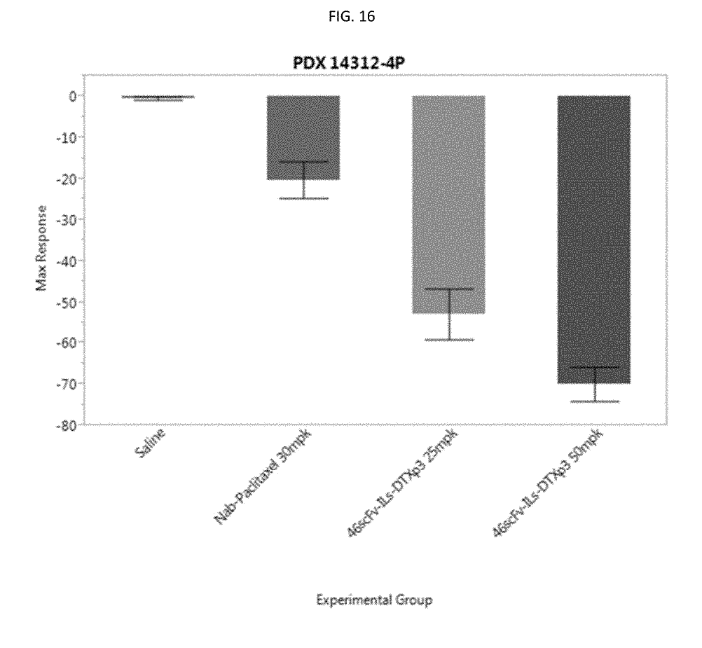

In terms of maximum response to treatment, 46scFv-ILs-DTXp3 at both dose levels proved superior to nab-Paclitaxel with the 25 mg/kg dosage exhibiting a 33% decrease in tumor volume while 50 mg/kg shows a 50% decrease compared to nab-Paclitaxel (FIG. 16). 46scFv-ILs-DTXp3 had a more potent anti-tumor activity than nab-Paclitaxel measured by maximum response and/or time to regrowth in PDX model 14312-4. FIG. 15 is a graph showing the time to regrowth for PDX 14312-49. FIG. 16 is a graph showing the maximum response for PDX 14312-4.

### Example 2B 12424 PDX Tumor Model

The #12424 PDX tumor model was described in Hylander (2005). The tumor material was collected from a 64 year old Caucasian male, who had been a life-long non-smoker. The cancer histological subtype was C25.7 (ICD-O-3 histology code 85033). The tumor was characterized as poorly differentiated, infiltrating ductal carcinoma, not otherwise specified with staging pT3, pN1 and M0. Histological staging per American Joint Committee on Cancer (5^{th} edition) was 2B. No follow-up treatment is available. The xenograft model was resistant to APO2L/Trail and to Gemcitabine treatment. The model had elevated levels of FGFR2 mRNA and was sensitive to Dovitinib (40 mg/kg) (Zhang et al., 2013). Model #12424 was maintained by passaging tumor fragments in immunodeficient mice. This PDX model was at passage 8 for study #12424-8P.

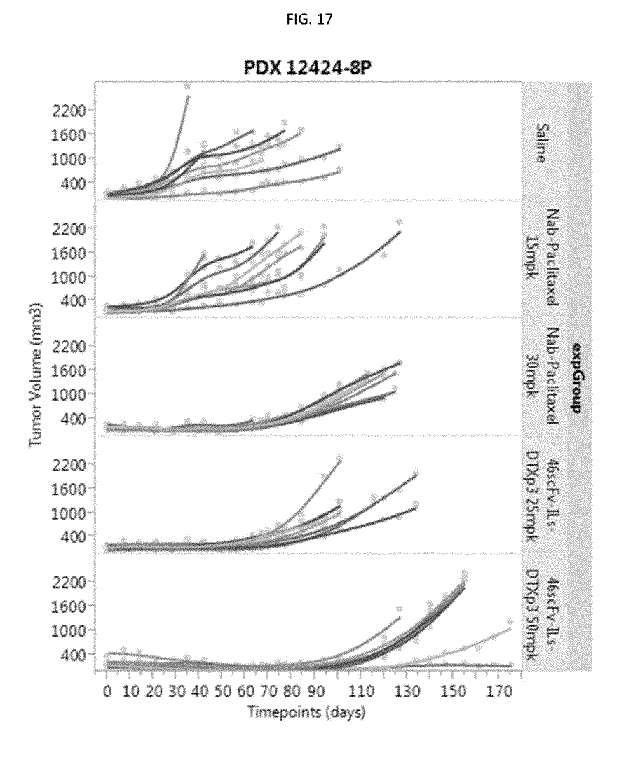

FIG. 17 is a graph showing the tumor growth curves for PDX 12424-8P

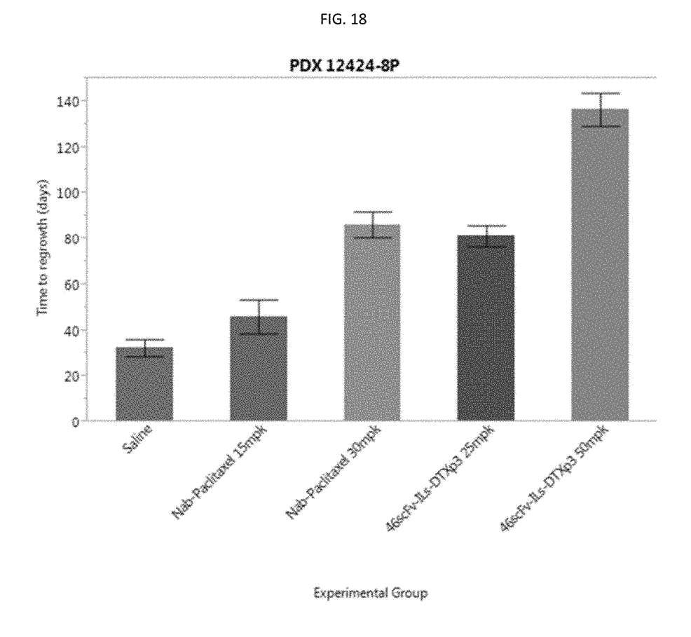

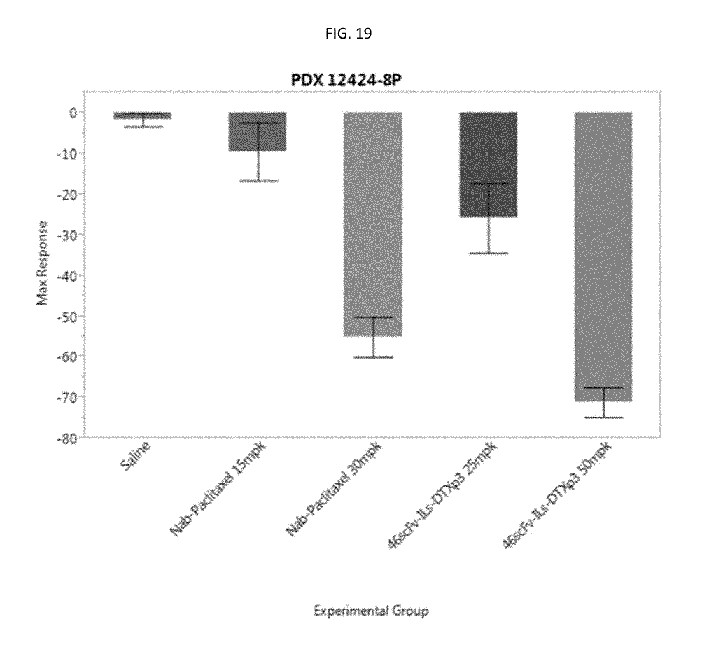

Results: Tumors treated with 15 mg/kg nab-Paclitaxel (n=8) had a mean tumor volume of 179±24 mm³ at treatment initiation. Tumors treated with 15 ng/kg nab-Paclitaxel increased steadily in size with no significant evidence of tumor growth inhibition as compared to saline control (FIG. 18 and FIG. 19). In comparison, tumors dosed with 30 mg/kg nab-Paclitaxel, with an initial tumor volume of 180±20 mm³, exhibited an increased time to regrowth (p=0.0003) and roughly 45% decrease in tumor volume when compared to 15 mg/kg nab-Paclitaxel.

Animals treated with either 25 mg/kg or 50 mg/kg of 46scFv-ILs-DTXp3 had an average tumor volume at treatment initiation of 143±14 mm³ and 196±32 mm³, respectively. Treatment of tumors in both dosage groups yielded significant inhibition of tumor regrowth when compared to saline control. The largest delay in tumor regrowth was observed in the 46scFv-ILs-DTXp3 50 mg/kg group, with an average delay in tumor regrowth of 104 days (p<0.0001) while the 25 mg/kg group delay was roughly half that (FIG. 18). Additional Tukey HSD analysis highlights statistically significant differences in mean time to regrowth between all cohorts with the exception of 15 mg/kg Nab-Paclitaxel/Saline and 30 mg/kg Nab-Paclitaxel/25 mg/kg 46scFv-ILs-DTXp3.

Overall, the two largest responses to treatment were the higher dose levels in both nab-Paclitaxel (30 mg/kg=53.4% decrease; p<0.0001) and 46scFv-ILs-DTXp3 (50 mg/kg=69.5% decrease; p<0.0001) when compared to saline control (FIG. 19). When compared directly to 30 mg/kg nab-Paclitaxel, 50 mg/kg treatment of 46scFv-ILs-DTXp3 did not demonstrate a significant advantage in terms of maximum response to drug (FIG. 19). However, in terms of inhibition of regrowth, 50 mg/kg 46scFv-ILs-DTXp3 outperformed both dose levels of nab-Paclitaxel (vs. 15 mg/kg nab-Paclitaxel=90 days, p<0.0001; vs. 30 mg/kg nab-Paclitaxel=50 days, p<0.0001) (FIG. 18). 46scFv-ILs-DTXp3 had a more potent anti-tumor activity than nab-Paclitaxel measured by maximum response and/or time to regrowth in PDX model #14242-8P (FIG. 18).

FIG. 18 is a graph showing the time to regrowth for PDX 12424-8P. FIG. 19 is a graph showing the maximum response for PDX 12424-8P.

### Example 2C: 15010 PDX Tumor Model

Pancreatic PDX model #15010, herein referred to as PDX 15010-5P, tumor tissue was collected from a 74 year old Caucasian female, who had been a life-long non-smoker. The tumor was located in the head of the pancreas (ICD-O-3 histology code 85033). The tumor was characterized as poorly differentiated, infiltrating ductal carcinoma, not otherwise specified with staging pT3, pN1 and M0. Histological staging per American Joint Committee on Cancer (6^{th} edition) was 2B (Hylander et al., 2013). The patient did not receive further therapy. Model #15010 was maintained by passaging tumor fragments in immunodeficient mice. At the time of implantation for the current study, this PDX model was at passage 5.

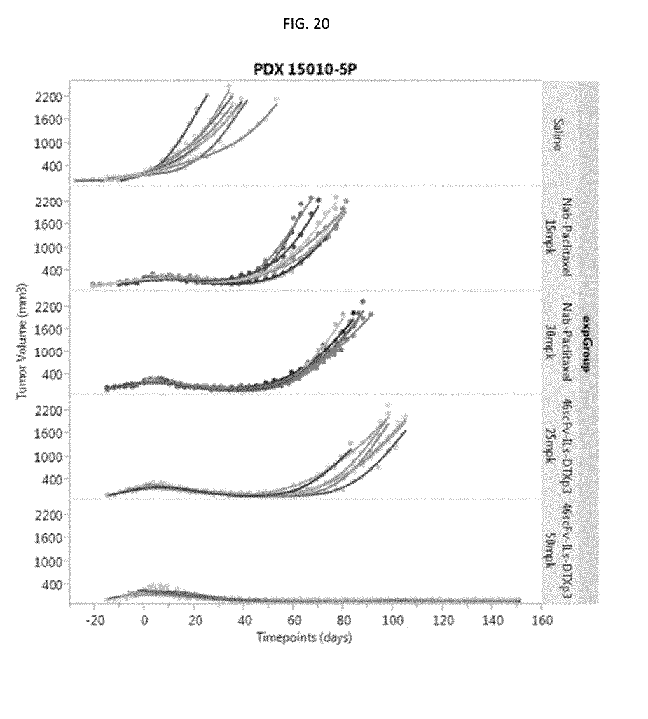

FIG. 20 is a graph showing tumor growth curves for Panc 15010-P5

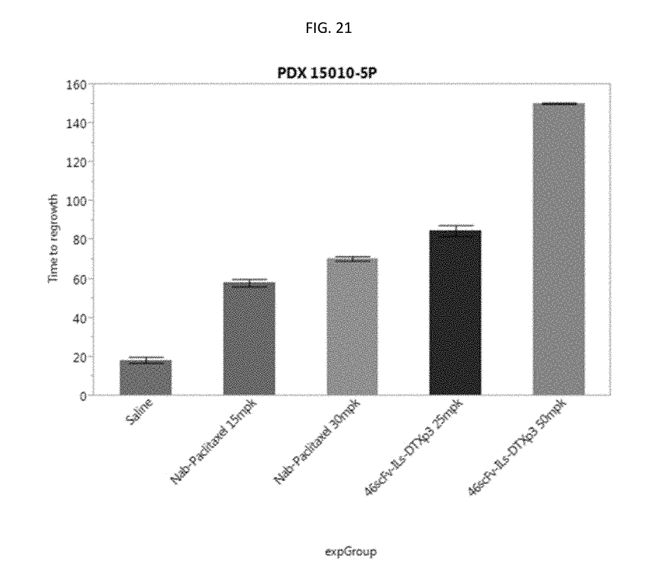

Initial tumor volumes for 15 mg/kg and 30 mg/kg nab-Paclitaxel were 212±6 mm³ and 229±11 mm³, respectively, at time of initial treatment. Both dose levels of nab-Paclitaxel (15 mg/kg=39.5, p<0.0001; 30 mg/kg=51.8, p<0.0001) and the 25 mg/kg 46scFv-ILs-DTXp3 dosage (64.7, p<0.0001) exhibited similar inhibition of tumor growth when compared to control (FIG. 21). However, the greatest inhibition of tumor regrowth was in the 50 mg/kg 46scFv-ILs-DTXp3 group, where it proved superior to control (131.6 days, p<0.0001), 25 mg/kg 46scFv-ILs-DTXp3 (66.9 days, P<0.0001) and the highest dose level of nab-Paclitaxel (79.8 days, P<0.0001) (FIG. 21).

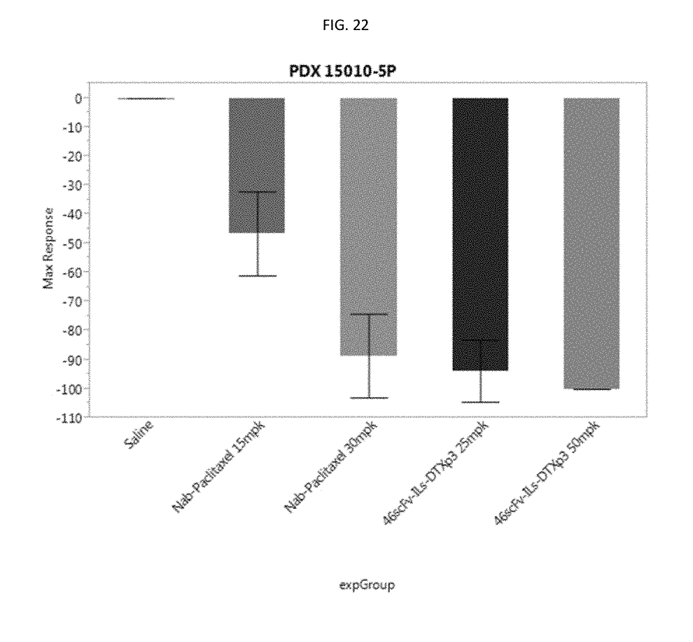

When comparing tumor maximal response to drug, both dose levels of 46scFv-ILs-DTXp3 (25 and 50 mg/kg) and 30 mg/kg nab-Paclitaxel demonstrated similar levels of response (90.5%, 100% and 88.5% respectively) compared to saline control group (FIG. 22). This stands in contrast to the 25 mg/kg nab-Paclitaxel group where all three treatment groups exhibit a statistically significant advantage in response (FIG. 22). 50 mg/kg 46scFv-ILs-DTXp3 had a more potent anti-tumor activity than nab-Paclitaxel measured by maximum response in PDX model 15010-P5. Looking at 50 mg/kg 46scFv-ILs-DTXp3's max response, it is again superior to nab-Paclitaxel, albeit with a minor advantage of 11.4% and was not statistically significant (p=0.2750).

FIG. 21 is a graph showing the time to regrowth for Panc 15010-P5. FIG. 22 is a graph showing the maximum response for Panc 15010-P5.

### Example 2D 14244 PDX Tumor Model

PDX model #14244 originated in the ampulla of Vater, also known as the hepatopancreatic duct, and is considered a relevant pancreatic model due to histology representative of pancreatic cancer (Sharma et al., 2014). This model has been shown to have elevated levels of FGFR2 mRNA (Zhang et al., 2013) and was sensitive to Apo2L/TRAIL treatment (Sharma et al., 2014). Growth from implantation occurred within 39 days and liver metastasis were found at 21 weeks. Model #14244 was maintained by passaging tumor fragments in immunodeficient mice. This PDX model was at passage 9 for study #14244-9P.

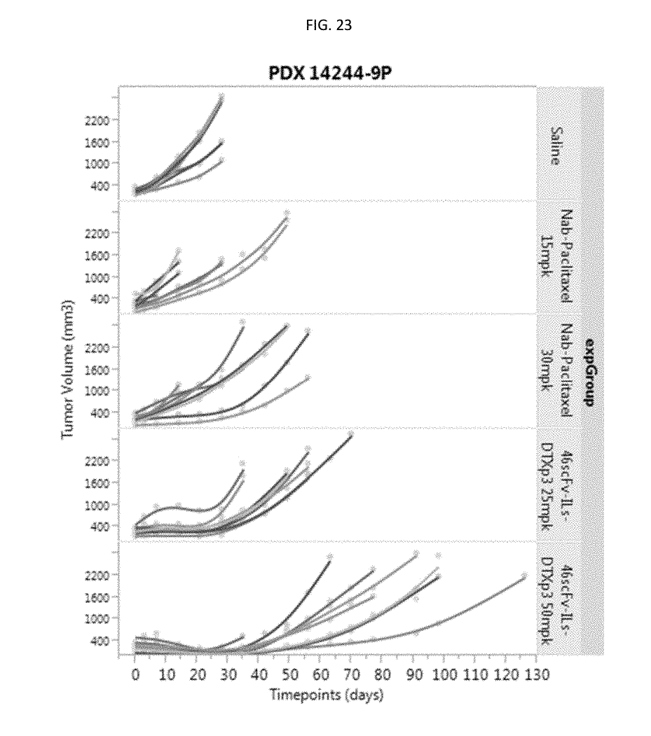

FIG. 23 is a graph showing the tumor growth curves PDC 14244-9P.

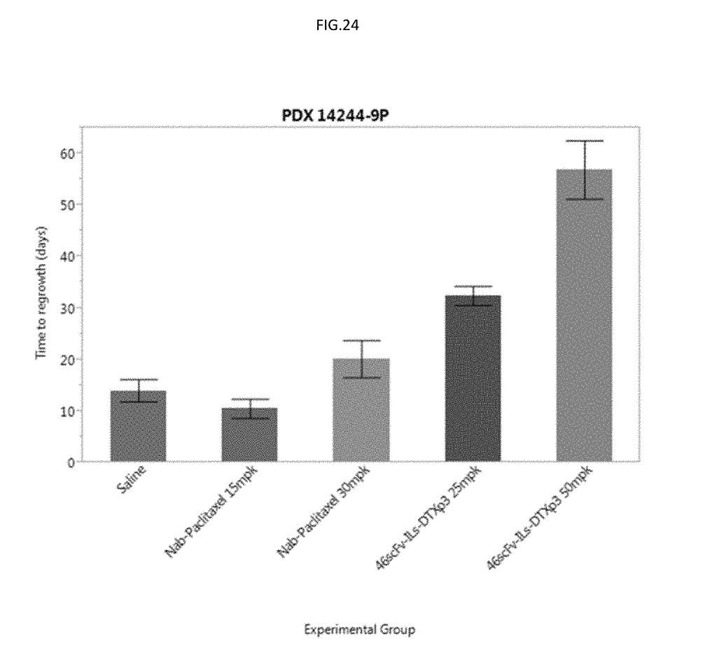

Both nab-Paclitaxel treatments (15 mg/kg, initial tumor volume=271±51; 30 mg/kg, initial tumor volume=250±50) did not exhibit significant inhibition of tumor growth compared to saline control (FIG. 23). In contrast, both 46scFv-ILs-DTXp3 25 mg/kg (18.4 days, p=0.0062) and 50 mg/kg (42.9 days, p<0.0001) showed inhibition of tumor growth compared to saline. Furthermore, 46scFv-ILs-DTXp3 inhibition increased in a dose dependent manner, with 50 mg/kg yielding a 24.5 day increase (p<0.0001) in time to regrowth compared to 25 mg/kg. Both dose levels of 46scFv-ILs-DTXp3, however, proved superior to the highest nab-Paclitaxel dose tested, 30 mg/kg, with 50 mg/kg 46scFv-ILs-DTXp3 inhibiting tumor regrowth by an additional 37 days (p<0.0001) and 35 mg/kg by another 12 days, but not reaching statistical significance (p=0.1014)(FIG. 24).

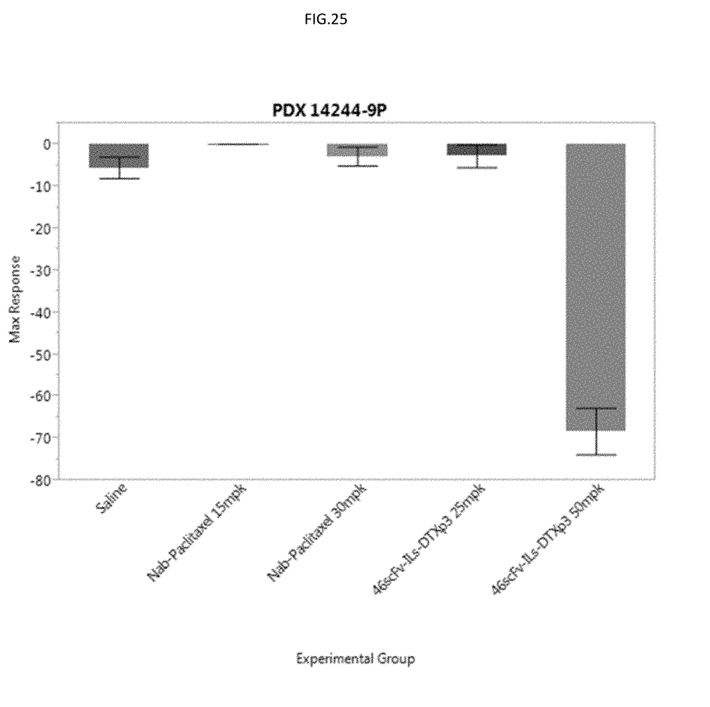

Regarding tumor response to treatment, only 46scFv-ILs-DTXp3 50 mg/kg showed any significant effect on tumor growth, with a 68% (p<0.0001) decrease in tumor proliferation compared to saline and 66% (P<0.0001) compared to the 30 mg·kg nab-Paclitaxel group (FIG. 25). All other conditions did not appear to significantly impede tumor growth (FIG. 24 and FIG. 25). 46scFv-ILs-DTXp3 had a more potent anti-tumor activitiy than nab-Paclitaxel measured by maximum response and/or time to regrowth in PDX model 14244-9P.

FIG. 24 is a graph showing the time to regrowth for PDX 14244-9P. FIG. 25 is a graph showing the maximum response for PDX 14244-9P.

### Example 3: Gemcitabine/nab-Paclitaxel (Abraxane) vs. Gemcitabine 46scFv-ILs-DTXp3

This study used a single pancreatic patient derived xenograft model, PDX 14244-10P, and three bladder patient derived models acquired from Jackson Laboratory (Bar Harbor, Me.), to test if the combination of Gemcitabine and 46scFv-ILs-DTXp3 would yield an increase in efficacy when compared to each drug alone and in the pancreatic model when compared to the current frontline pancreatic combination of Gemcitabine+nab-Paclitaxel.

In a comparison between monotherapy of both Gemcitabine and 46scFv-ILs-DTXp3, the combination of Gemcitabine and 46scFv-ILs-DTXp3 proved to be superior as measured by tumor growth inhibition and maximal tumor response to drug. Furthermore, when compared to the Gemcitabine/nab-Paclitaxel therapy, Gemcitabine/46scFv-ILs-DTXp3 also demonstrated superiority in both maximum response and time to regrowth.

### Example 3A 14244 PDX Tumor Model

PDX model #14244 originated in the ampulla of Vater, also known as the hepatopancreatic duct, and is considered a relevant pancreatic model due to histology representative of pancreatic cancer (Sharma et al., 2014). This model has been shown to have elevated levels of FGFR2 mRNA (Zhang et al., 2013) and was sensitive to Apo2L/TRAIL treatment (Sharma et al., 2014). Growth from implantation occurred within 39 days and liver metastasis were found at 21 weeks. Model #14244 was maintained by passaging tumor fragments in immunodeficient mice. This PDX model was at passage 10 for study #14244-10P.

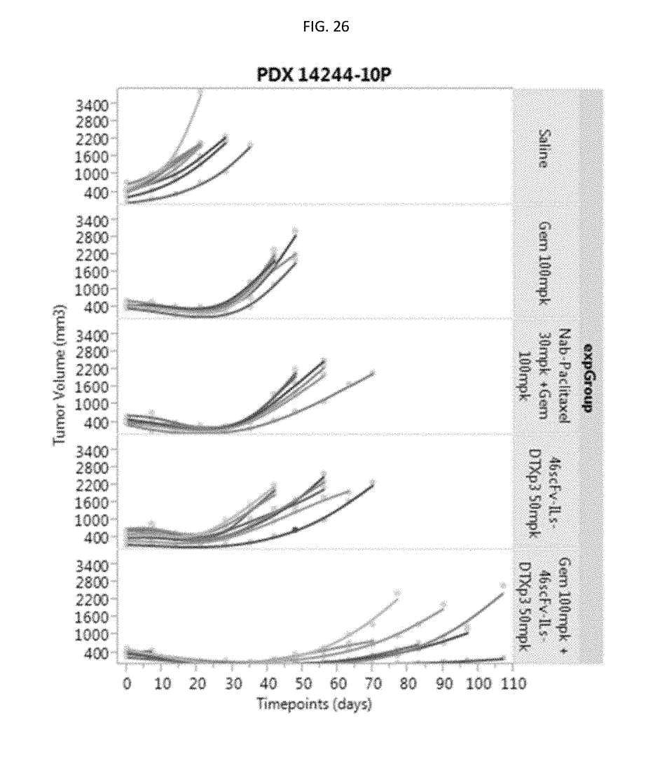

FIG. 26 is a graph showing tumor growth curves for PDX 14244-10P

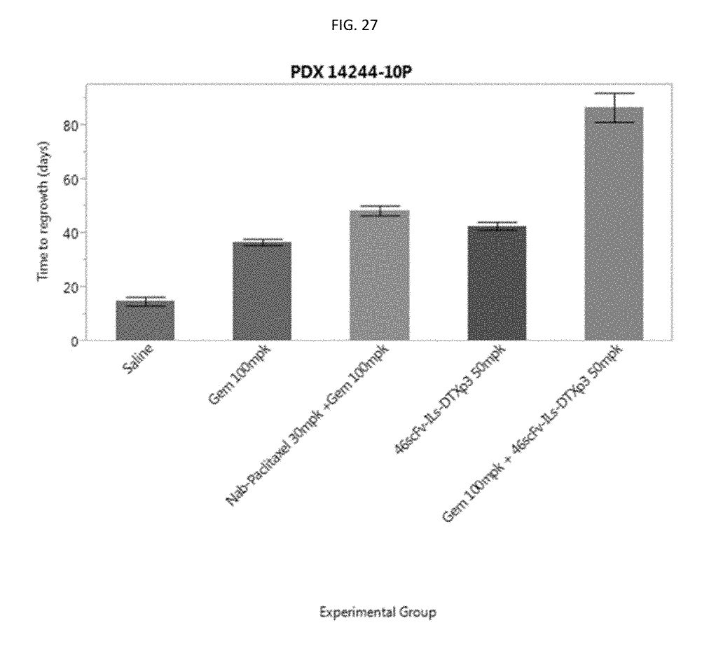

Results: Mean tumor volume across treatment groups at time of treatment initiation were roughly equivalent (range=419 to 437 mm³). In all chemotherapy treated models, there were varying levels of tumor growth inhibition observed (FIG. 26). Quantification of time to regrowth, defined as the amount of time takes for a tumor to reach four times initial tumor volume at day 0, showed similar growth inhibition between control, 100 mg/kg Gemcitabine (21.9 days), 50 mg/kg 46scFv-ILs-DTXp3 (27.8 days) and the combination of 30 mg/kg nab-Paclitaxel+100 mg/kg Gemcitabine (33.5 days) (FIG. 27). When comparing these treatment cohorts to each other, there is no statistically significant advantage in tumor inhibition between Gemcitabine/46scFv-ILs-DTXp3 mono-therapies (5.9 days, p=0.4834) or between the combination therapy of 50 mg/kg nab-Paclitaxel+100 mg/kg Gemcitabine and 50 mg/kg 46scFv-ILs-DTXp3 (5.7 days, p=0.5849). However, the comparison of time to regrowth between 50 mg/kg nab-Paclitaxel+100 mg/kg Gemcitabine and 100 mg/kg Gemcitabine showed minor difference of 11.5 days (p=0.0392).

The greatest overall inhibition of tumor growth was found to be in the 100 mg/kg Gemcitabine+50 mg/kg 46scFv-ILs-DTXp3 (FIG. 27). Indeed, the Gemcitabine/46scFv-ILs-DTXp3 combination was superior to both monotherapy treatments of the combination's constituent components (49.96 days, p<0.0001 vs. Gemcitabine and 44 days, p<0.0001 vs. 46scFv-ILs-DTXp3). When compared to the combination therapy of 30 mg/kg nab-Paclitaxel+100 mg/kg Gemcitabine, the 46scFv-ILs-DTXp3+Gemcitabine combination was clearly superior, inhibiting tumor regrowth for an additional 38.38 days (p<0.0001).

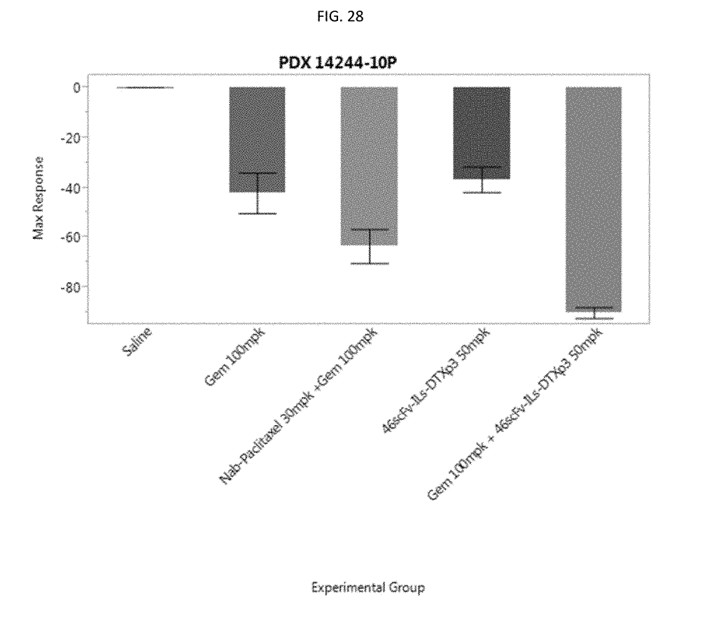

FIG. 27 is a graph showing the time to regrowth for PDX 14244-10P. FIG. 28 is a graph showing the maximum response for PDX 14244-10P.

Using a secondary metric for efficacy, maximal response to therapy, we observe that both monotherapies of Gemcitabine and 46scFv-ILs-DTXp3 exhibit similar tumor responses, 42.11 (p<0.0001) and 36.66% (p=0.0002) respectively (FIG. 28). Both monotherapy responses are surpassed by the nab-Paclitaxel/Gemcitabine combo; with this combination proving superior to 46scFv-ILs-DTXp3 (26.8%, p=0.0141) and Gemcitabine monotherapy (21.4%, p=0.0729), although the Gemcitabine comparison did not meet statistical significance.

The greatest overall tumor response to drug was seen in the 100 mg/kg+50 mg/kg 46scFv-ILs-DTXp3 combination, with a 90.33% (p<0.0001) decrease in tumor volume when compared to control. In addition, the combination of Gemcitabine/46scFv-ILs-DTXp3 also outperformed both monotherapies (vs. 100 mg/kg Gemcitabine=53.68, P<0.0001 and vs. 50 mg/kg 46scFv-ILs-DTXp3=48.22, p<0.0001), as well as the combination of nab-Paclitaxel/Gemcitabine with a 26.84% (p=0.0181) improvement of tumor response to drug (FIG. 28).

### Example 3B Bladder PDX Tumor Models

Immunodeficient mice-bearing tumor models BL-0382, BL-0293 and BL-0440 were acquired from Jackson Laboratory and randomized into the following experimental groups: Saline, Gemcitabine, 46scFv-ILs-DTXp3, Gemcitabine/46scFv-ILs-DTXp3. 46scFv-ILs-DTXp3 was treated at 25 mg/kg DTX equivalent I.V. weekly for four weeks and Gemcitabine was dosed at 75 mg/kg I.V. for model BL-0293 and 150 mg/kg I.V. for models BL-0382 and BL-0440. For the Gemcitabine/46scFv-ILs-DTXp3 doses used for the monotherapy arms were combined and dosed in the same day.

The animals received four tail vein injections, at the intervals of 7 days. Tumor size was monitored once to twice weekly. The tumor progression was monitored by palpation and caliper measurements of the tumors along the largest (length) and smallest (width) axis twice a week. The tumor sizes were determined twice weekly from the caliper measurements using the formula (Geran, R. I., et al., 1972 Cancer Chemother. Rep. 3:1-88):

Tumor volume(TV)=[(length)×(width)²]/2

Maximum response was calculated using the following formula where TV is tumor volume:

Max tumor regression=[(TVₘᵢₙ−TV_{day0})/TV_{day0}]×100

Maximum tumor regression was classified as complete tumor regression (100% regression with no palpable tumor), partial tumor regression (max tumor regression more than 30%) or no tumor regression.

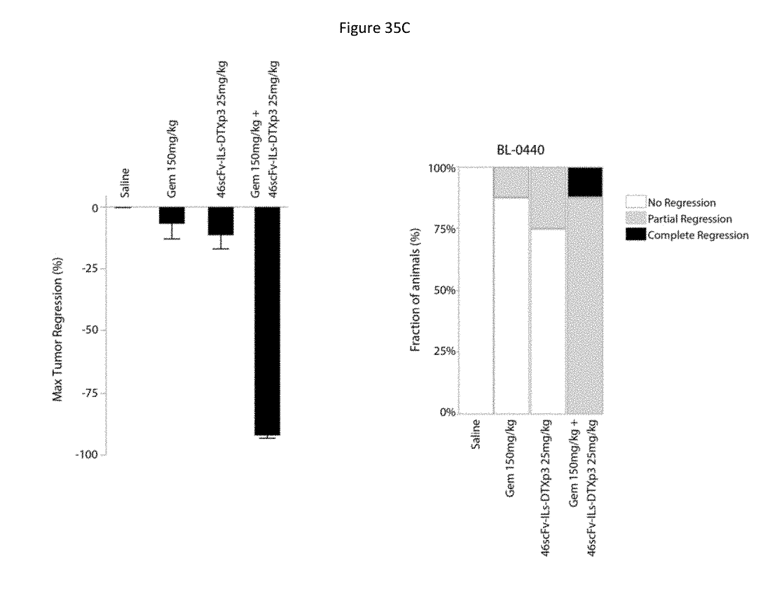

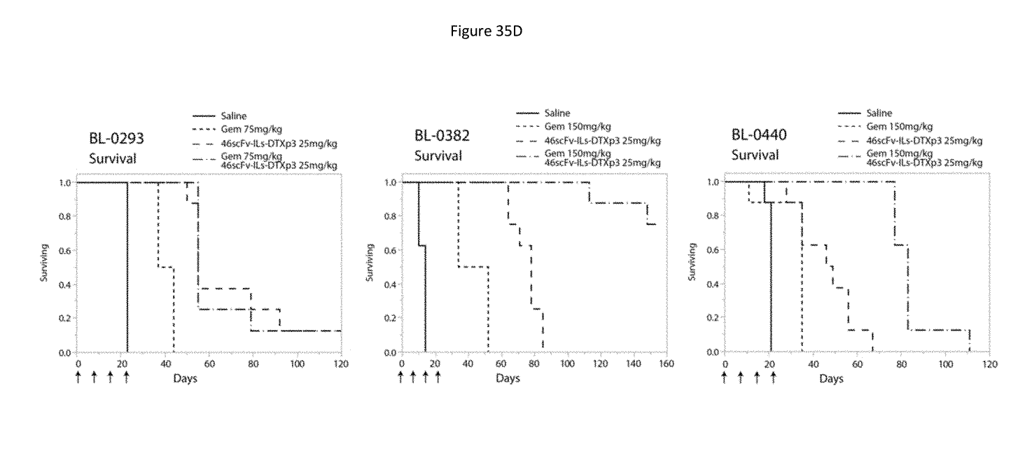

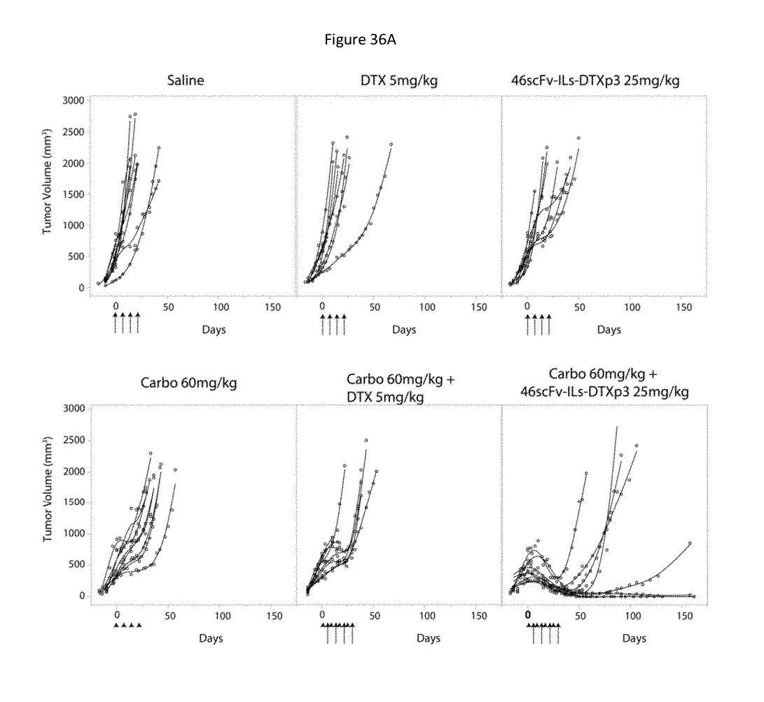

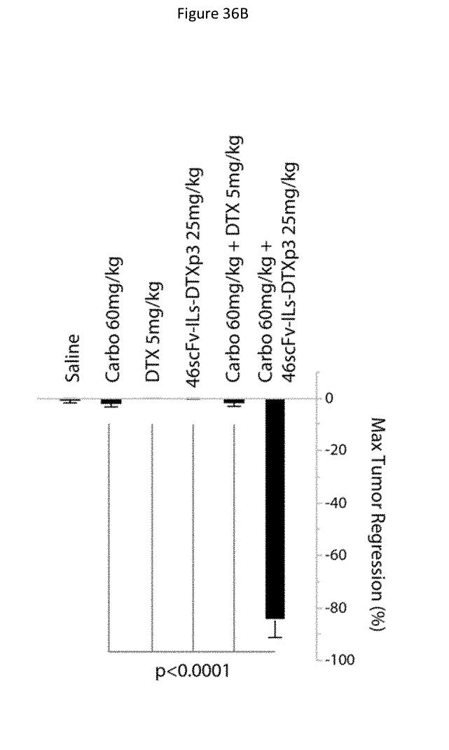

In all three models, the combination Gemcitabine/46scFv-ILs-DTXp3 was superior than the monotherapy groups determined either by the induction of more pronounced tumor regression (FIG. 35A-C) and/or extension of survival (FIG. 35D).

### Example 4: Carboplatin/docetaxel vs. Carboplatin/46scFv-1 Ls-DTXp3

Immunodeficient mice were implanted with human ovarian patient derived model OVx-132. Animals randomized into the following experimental groups: Saline, docetaxel at 5 mg/kg, 46scFv-ILs-DTXp3 at 25 mg/kg, carboplatin 60 mg/kg, carboplatin/docetaxel, and carboplatin/46scFv-ILs-DTXp3. For the combinations groups carboplatin/docetaxel and carboplatin/46scFv-ILs-DTXp3 doses used for the monotherapy arms were combined and dosed 3 days a part with carboplatin being dosed first followed by the docetaxel or 46scFv-ILs-DTXp3.

The animals received four tail vein injections, at the intervals of 7 days. Tumor size was monitored once to twice weekly. The tumor progression was monitored by palpation and caliper measurements of the tumors along the largest (length) and smallest (width) axis twice a week. The tumor sizes were determined twice weekly from the caliper measurements using the formula (Geran, R. I., et al., 1972 Cancer Chemother. Rep. 3:1-88):

Tumor volume(TV)=[(length)×(width)²]/2

Maximum response was calculated using the following formula where TV is tumor volume:

Max tumor regression=[(TVₘᵢₙ−TV_{day0})/TV_{day0}]×100

Maximum tumor regression was classified as complete tumor regression (100% regression with no palpable tumor), partial tumor regression (max tumor regression more than 30%) or no tumor regression.

Animals were monitored until tumor regrowth or end of monitoring period (>120 days). Time to regrowth was defined as time for tumor to double its volume. Animals sacrificed prior to tumor volume doubling are censored.

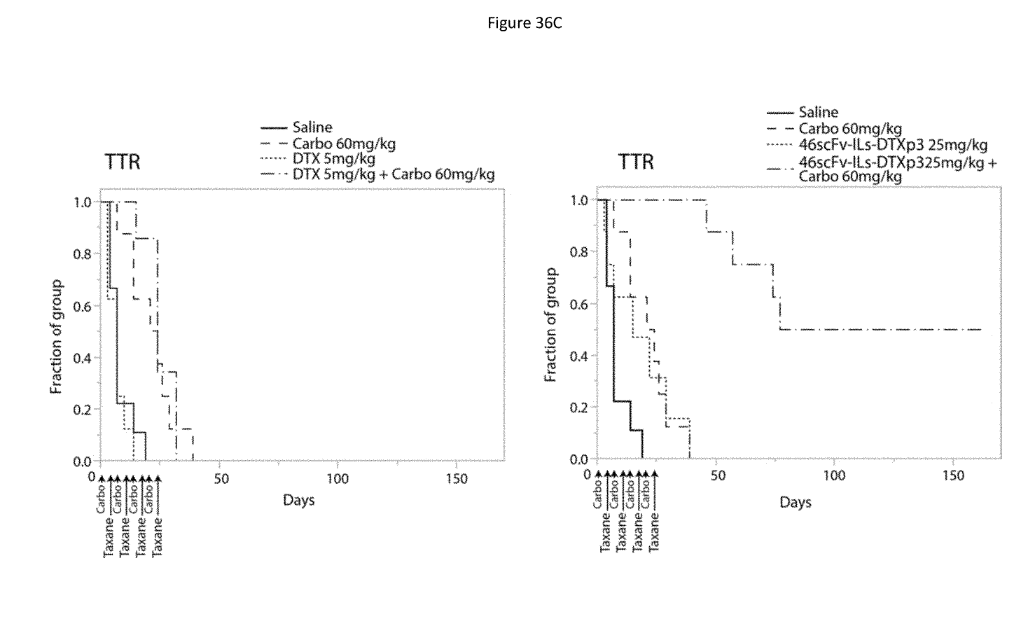

The growth curves shown below illustrate the treatment effect when 46scFv-ILs-DTXp3 was combined with carboplatin (FIG. 36A). Carboplatin, as well as 46scFv-ILs-DTXp3 and docetaxel showed minimal growth arrest and no tumor regression when given as monotherapies. A combination of carboplatin and docetaxel did increase the growth arrest in comparison to the monotherapies, but did not induce any tumor regression. However, a combination of 46scFv-ILs-DTXp3 and carboplatin significantly increased the tumor doubling time, in addition to inducing regression in 100% of treated animals. In this study two mice achieved complete responses with no residual tumor burden (FIG. 36B).

In the combination part of the study, animals were monitored for an extensive period of time post treatment interruption (160 days). FIG. 36C shows Time-to-tumor regrowth (TTR) of all treatment groups. Docetaxel, carboplatin, 46scFv-ILs-DTXp3 monotherapy and carboplatin/docetaxel combination induced minor delay on time to regrowth. This contrasted with the significant TTR delay seen in the carboplatin/46scFv-ILs-DTXp3 combination arm which induced significant delay in median TTR. Of note 50% of the animals treated with carboplatin/46scFv-ILs-DTXp3 showed durable response with no tumor regrowth for 3 months post treatment interruption.

### Example 5: Tolerance Test of 46scFv-ILs-DTXp3 with Gemcitabine or Carboplatin

This short-term tolerance test of 46scFv-ILs-DTXp3 and Gemcitabine or Carboplatin is to determine the tolerated dose and optimal dose scheduling for the purpose of minimizing toxicity. Gemcitabine and Carboplatin are chemotherapeutic agents likely to be combined with 46scFv-ILs-DTXp3 in the clinic. Gemcitabine alone is well tolerated in mice; most protocols list an MTD for i.p. dosing around 240 mpk q3d. We did not find toxicity (as observed as weight loss or catalyst enzyme profiling different than control mice) following an i.v. dose for 10 days with doses as high as 291.6 mg/kg. The stock concentration of 46scFv-ILs-DTXp3 was 11.09 mg/ml. Carboplatin was purchased from Hospira Inc and used at a stock concentration of 10 mg/ml. Gemcitibine was purchased from Sun Pharam and used at a stock concentration of 38 mg/ml.

CD-1 female mice (7-8 week old) were obtained from Charles River. During the treatment phase mice body weight was monitored daily. In order to assess the effect of drug scheduling on tolerability, animals were treated with a single dose of 46scFv-ILs-DTXp3 followed by a dose of either carboplatin or gemcitabine at various dose levels and starting at different timepoints post 46scFv-ILs-DTXp3 treatment (Table 3). Additionally, a single treatment group treated with carboplatin or gemcitabine was added.

TABLE 3
Experimental Design

| | 46scFv-ILs-DTXp3 | Carboplatin | Gemcitabine |
|---|---|---|---|
| Dose | 100 mpk | 63, 72, 84 mpk | 162, 214, 292 mpk |
| Time points | 0 h | 8 h, 24 h, 72 h | 8 h, 24 h, 72 h |

At study end (10 days after last drug dose, i.e. day 10 for 8 h, day 11 for 24 h, and day 13 for 72 h combinations), mice were euthanized with carbon dioxide. Blood was collected by cardiac puncture. Catalyst analysis used an EQUINE-15 clip with an additional individual ALT assay added (Idexx, Westbrook, Mass.). Tissues collected included liver, kidney, spleen, heart, skeletal muscle (with skin attached). Tissues were fixed in 10% neutral buffered formalin ˜24 h, then stored in 70% ethanol. Tissues from mice in the highest-dose groups (all time points) as well as untreated and single-agent controls were shipped to Mass Histology Inc (Worcester, Mass.) for processing, and H&E staining of sectioned tissues. Received slides were scanned at 20X on the aperio bright field scanner.

### Example 5A: 46scFv-ILs-DTXp3 In Combination With Carboplatin

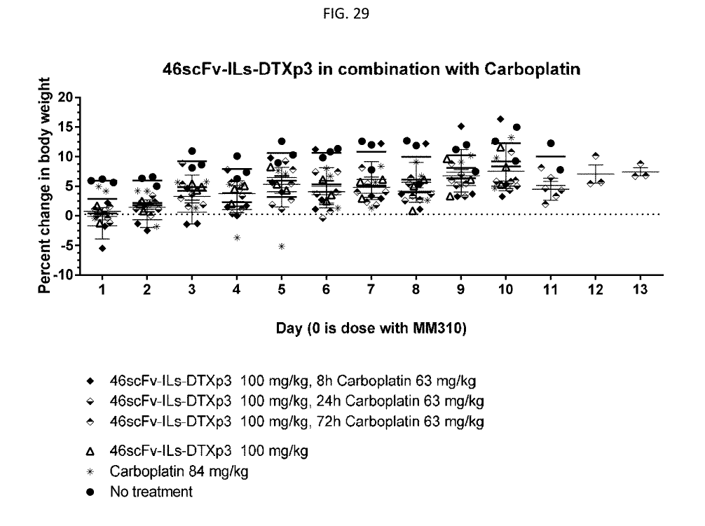

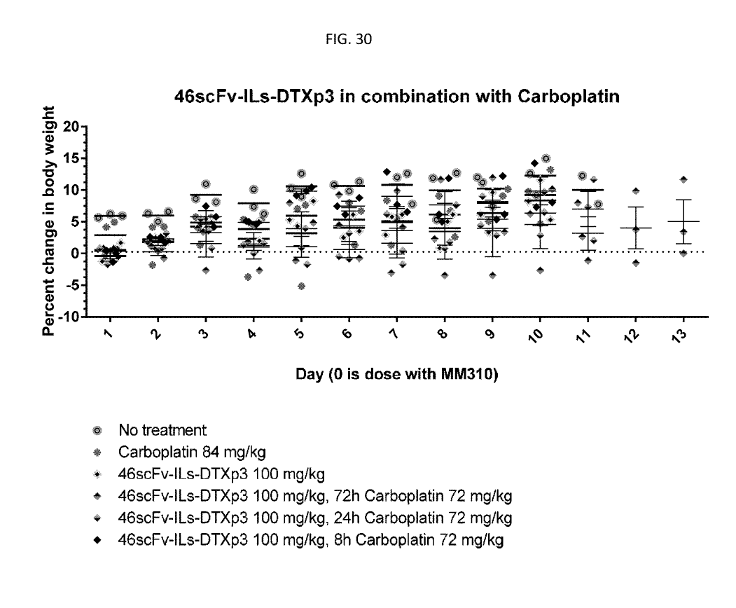

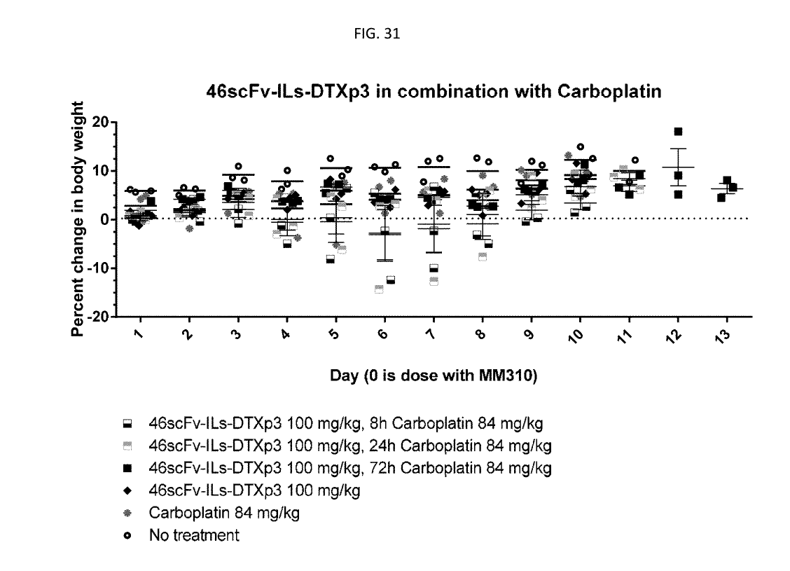

Individual points are shown for each measurement along with a line at the mean and error bars for SEM in FIG. 29: 63 mg/kg Carboplatin, FIG. 30: 72 mg/kg Carboplatin, FIG. 31: 84 mg/kg Carboplatin. Data is shown with Day 0 as the date that the 46scFv-ILs-DTXp3 was given; additional prism files attached are set for day 0 to be the first dose of Carboplatin.

### Example 5B: 46scFv-ILs-DTXp3 In Combination With Gemcitabine

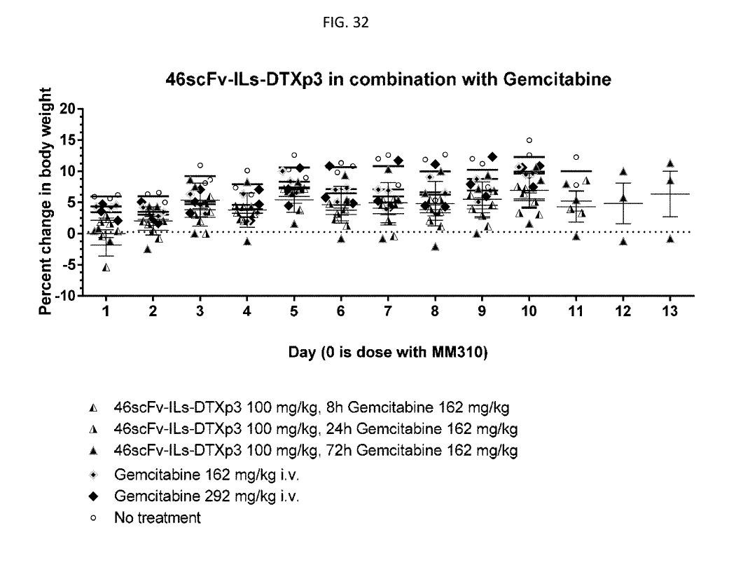

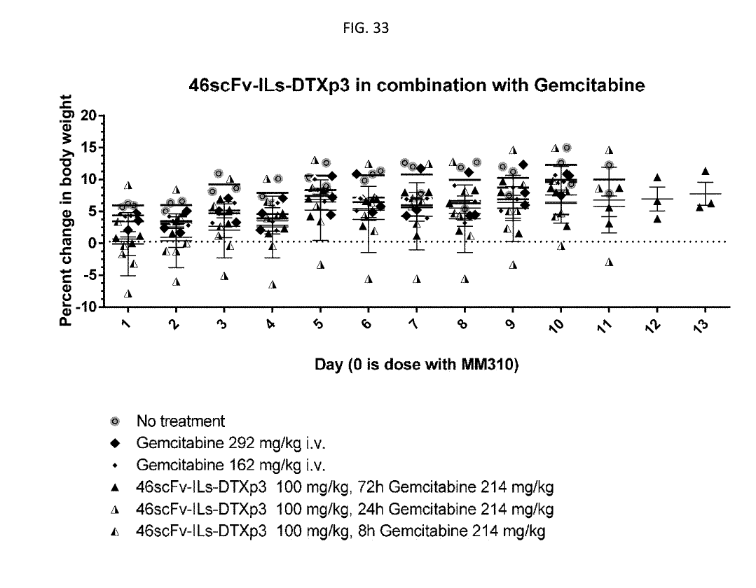

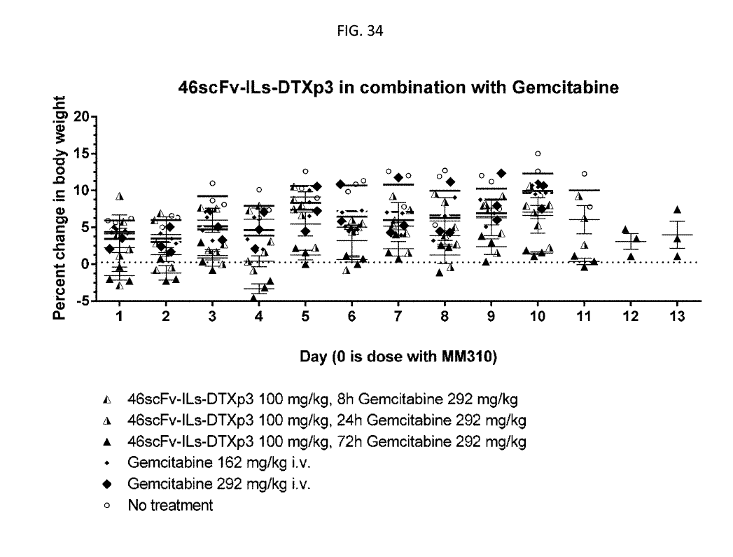

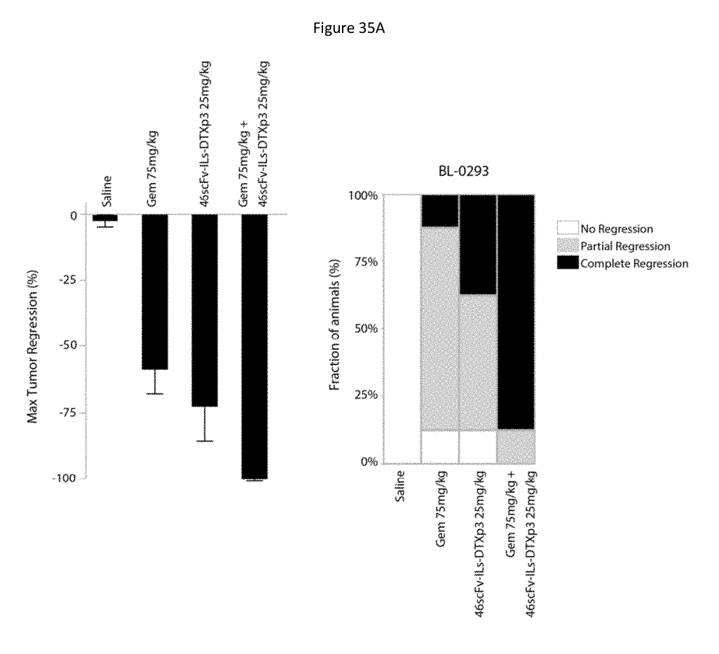

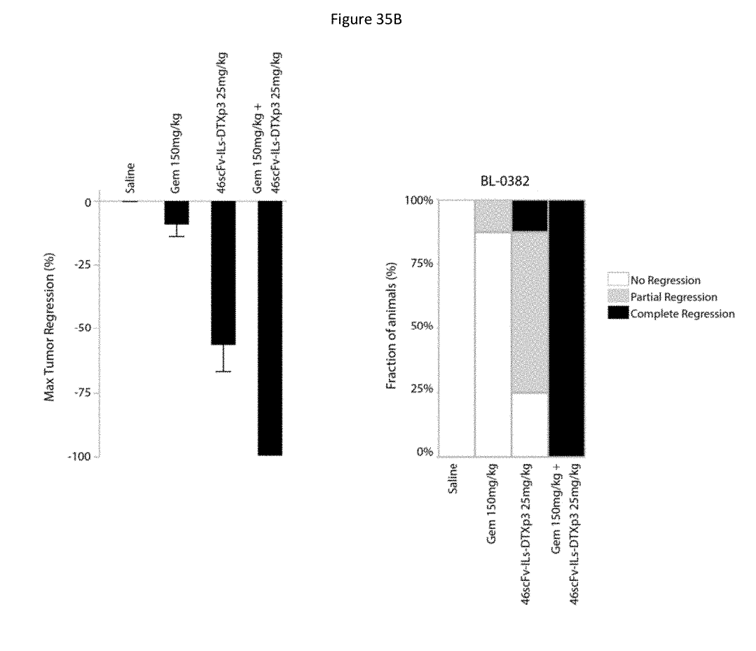

Individual points are shown for each measurement along with a line at the mean and error bars for SEM in FIG. 32: 162 mg/kg Gemcitabine; FIG. 33: 214 mg/kg Gemcitabine; and FIG. 34: 292 mg/kg Gemcitabine. Data is shown with Day 0 as the date that the 46scFv-ILs-DTXp3 was given; additional prism files attached are set for day 0 to be the first dose of Gemcitabine.

Catalyst profiling for control mice and 46scFv-ILs-DTXp3 in combination with Gemcitabine was performed.

Treatment related effects include: increased incidence of individual hepatocyte necrosis (minimal to mild) in carboplatin (combo and mono) and gemcitabine (combo) treated groups. This is minimal and likely reversible. There is also an increase in mitotic rate in the liver of carboplatin treated groups. This is likely regenerative (reparative) and reversible. Increased extramedullary hematopoieisis (EMH) in the spleen of carboplatin (mono and combo) and Gemcitabine (combo). This is likely a response to effects on bone marrow and is regenerative in nature. However, this cannot be confirmed without bone marrow or CBC data. Significant pathology which does not appear to be treatment related: Granulomatous hepatitis and histiocytic infiltrates in the spleen in C10/M3 46scFv-ILs-DTXp3 only and in C23/M2 Gemcitabine mono this granulomatous/histiocytic inflammation is unknown but does not appear to be treatment related. Foci of necrosis is liver (greater than single or individual cell necrosis seen in all treatment groups) seen in C7/M3 46scFv-ILs-DTXp3/carboplatin control. This lesion does not appear to be treatment related.

The key findings from this study were:
No mice lost >20% BW in the study (acute tox study, single dose with 46scFv-ILs-DTXp3 at day 0, single follow-up dose, end 10 days after last drug).
Carboplatin 84 mg/kg groups dosed at 8 h, 24 h showed at most 10% weight loss in individual mice at day 6 but these regained weight by day 10-11.
Catalyst profiles for most mice look similar to untreated age-matched untreated controls, no obvious trend for abnormal enzyme activity with 46scFv-ILs-DTXp3 in combination with carboplatin vs. carboplatin alone.
Gemcitabine 292 mg/kg group 72 h post-46scFv-ILs-DTXp3 show least growth of the 292 mg/kg groups and elevated ALT and AST at study end.
Pathologist review of highest-treated groups and controls found minimal treatment related effects and some significant pathology which did not appear to be treatment related.

### Example 6: Synthesis of Docetaxel Prodrugs

Docetaxel prodrugs of formula (I) can be prepared by a various reaction methods, including the reaction scheme shown in FIG. 2A. Representative synthetic examples of two compounds are provided below. These or other docetaxel prodrugs, and various pharmaceutically acceptable salts thereof, can be prepared by various suitable synthetic methods. The Table in FIG. 2B provide a representative list of examples of certain docetaxel prodrugs.

### Example 6A: Synthesis of 2′-O-(4-Diethylamino Butanoyl) DTX Hydrochloride (Compound 3)

Docetaxel (DTX) (0.25 g, 0.31 mmol), 4-diethylamino butyric acid hydrochloride (0.12 g, 0.62 mmol), EDAC.HCl (0.12 g, 0.62 mmol), and DMAP (0.08 g, 0.62 mmol) were all weighed into a 15 mL vial under Ar. To this 6 mL of anhydrous DCM was added at rt under Ar and stirred at rt for 18 h. HPLC after 18 h stirring shows 43% product with 57% DTX remaining unreacted. Additional amount of 4-diethylamino butyric acid hydrochloride (0.15 g, 0.77 mmol) was added and stirring continued for additional 24 h. HPLC shows 91% product with 8% DTX remaining unreacted. The reaction was stopped and directly loaded on a 12 g cartridge and FCC was performed using 3-12% MeOH/CHCl3. Fractions 30-49 were pooled together, 0.5 mL of 0.05N HCl in 2-propanol was added and evaporated under 30° C. to give 0.19 g of a white solid (63% yield).

HNMR:δ=8.11 (d, 2H), 7.65-7.57 (m, 1H), 7.56-7.46 (m, 2H), 7.45-7.29 (m, 5H), 6.29-6.12 (m, 1H), 5.99 (d, 1H), 5.68 (d, 1H), 5.58-5.40 (m, 1H), 5.31 (d, 1H), 5.24 (s, 1H), 4.96 (d, 1H), 4.38-4.08 (m, 5H), 3.91 (d, 1H), 3.20-2.30 (m, 3H), 2.70-2.52 (m, 2H), 2.50-2.42 (m, 2H), 2.25-2.05 (m, 3H), 1.94 (s, 3H), 1.92-1.78 (m, 2H), 1.75 (s, 3H), 1.50-1.35 (m, 9H), 1.32 (s, 9H), 1.30-1.20 (m, 6H), 1.12 (s, 3H) ppm. ppm. MS (ESI) m/z: 949.4 [M]+

### Example 6B: Synthesis of 2′-O[4-(N,N-Dimethylamino) butanoyl)]DTX Hydrochloride (Compound 4)

Docetaxel (DTX) (0.28 g, 0.34 mmol), N,N-dimethylaminobutyric acid hydrochloride (0.07 g, 0.43 mmol), EDAC.HCl (0.13 g, 0.69 mmol) and DMAP (0.05 g, 0.41 mmol) were all weighed into a 15 mL vial under argon. To this 7 mL of anhydrous DCM was added and the mixture was stirred at rt for 18 h. HPLC after 18 h shows 94% product with 3% byproduct and 3% of DTX remaining. The reaction residue was directly loaded on a 12 g cartridge and FCC was performed using 5-50% 2-propanol/CHCl3. Fractions 22-41 were pooled together, 0.5 mL of 0.05N HCl in 2-propanol was added and evaporated under 40° C. to give a white solid weighing 0.16 g (48% yield). Relatively low yield despite good conversion of DTX to product was presumably due to the poor solubility of the product in 2-propanol.

1H NMR (300 MHz, CDCl3+(CD3)250): δ=8.02 (d, 2H), 7.60-7.42 (m, 6H), 7.38-7.25 (m, 4H), 7.24-7.12 (m, 1H), 6.92 (d, 1H), 6.40-5.88 (m, 1H), 5.53 (d, 1H), 5.35-5.21 (m, 1H), 5.20-5.08 (m, 2H), 4.85 (d, 1H), 4.74 (d, 1H), 4.26 (s, 1H), 4.13 (dd, 3H), 3.74 (d, 1H), 3.56 (s, 1H), 3.16-2.74 (m, 3H), 2.64-2.48 (m, 7H), 2.49-2.00 (m, 5H), 1.84-1.68 (m, 4H), 1.62 (m, 3H), 1.30 (s, 9H), 1.07 (s, 3H), 1.02 (s, 3H) ppm. MS (ESI) m/z: 921.4 [M]+

### Example 7. Procedure for Hydrolysis in Buffer

A volume of a 10 mg/mL solution of drug in DMSO as needed to provide the desired concentration (typically 16 μL to yield a 80 μg/mL solution) (see Table 1) is placed in a glass test tube or 4 mL vial. Additional DMSO may also be added (e.g. 64 μL when using 16 μL of drug solution) to yield a final total DMSO concentration of 4%. The DMSO solutions are mixed by brief vortexing, and then 2 mL (20 mM) HEPES buffer for pH 7.5 and 2 mL of (20 mM) phosphate buffer for pH 2.5 is added and the mixture is vortexed again. The initial pH may be adjusted by addition of HCl or NaOH. The use of 20 mM buffer was found to provide better pH control and to avoid pH drift during incubation.

Then 100 μL aliquots of the buffer solution of drug are transferred into HPLC vials and incubated in a 37° C. water bath. The remaining solution is also incubated at 37° C. in 4 mL vials for monitoring of pH.

At each time point 900 μL of 0.1% trifluoroacetic acid (TFA) in acetonitrile (ACN) is added to the HPLC vial and the contents are vortexed. The vials are then placed in an autosampler rack at 4° C. for HPLC analysis.

Time zero data points are typically obtained from a solution of 4 μL DMSO stock in 5 mL of 0.1% TFA/ACN (8 μg/mL).

HPLC analysis is performed on a SYNERGI 4 micron Polar RP-80A, 250×4.6 mm column, using a flow rate of 1 mL./min, a 50 μL injection volume, column temperature of 25° C. and with UV detection at 227 nm. Most compounds are analyzed using a 13 min gradient (Method A) from 30 to 66% acetonitrile in aqueous 0.1% TFA, followed by a 1 min gradient back to 30% and a hold at 30% for 6 minutes. If the retention time is too long for this method, a 20 min gradient (Method B) of 30 to 90% acetonitrile, followed by a 1 min return to 30% and held for 9 min at 30% is employed.

The extent of hydrolysis is reported in Table 4 below as the % PTX or DTX formation based on relative peak areas.

TABLE 4

| Com- | | Percentage of Hydrolysis at Different Time Points (h) | | | | | |
|---|---|---|---|---|---|---|---|
| pound | pH | 0 | 2.5 | 5 | 24 | 48 | 96 |
| Com- | 2.5 | 0.00 | n/a | 0.00 | 0.00 | 0.00 | 0 |
| pound 1 | 7.5 | 0.00 | n/a | 23.41 | 51.38 | 69.00 | 88 |
| Com- | 2.5 | 0.00 | n/a | 0.00 | 0.00 | 0.00 | 0 |
| pound 3 | 7.5 | 0.00 | n/a | 45.76 | 100.00 | 100.00 | 100 |
| Com- | 2.5 | 0.00 | n/a | 0.00 | 0.00 | 0.00 | 0 |
| pound 4 | 7.5 | 0.00 | n/a | 76.07 | 100.00 | 100.00 | 100 |

### Example 8. Procedure for Hydrolysis in Buffered Plasma

Human plasma (HP 1055 from Valley Biomedical Inc, Winchester, Va.; pooled human plasma preserved with Na citrate) is centrifuged to remove precipitate. To 1.5 mL centrifuge tubes is added 0.9 mL of plasma and 40-50 μL of pH 7.5, 0.9 M HEPES buffer (final concentration of 40-50 mM and pH of 7.5). This is mixed by inversion, and then the tubes are warmed to 37° C. Then 7.2 μL of a 10 mg/mL DMSO solution of drug is added (80 μg/mL final concentration) and the contents mixed by inversion. The solution in plasma is then aliquoted into 1.5 mL centrifuge tubes and placed in a 37° C. bath. At each time point 900 μL of 0.1% trifluoroacetic acid (TFA) in acetonitrile (ACN) is added to the tubes. The contents are vortex mixed and then centrifuged for 5 min at 13,000 rpm. The supernatants are analyzed by HPLC as described under Procedure for Hydrolysis in Buffer. Time zero data points are obtained from a solution of 4 μL DMSO stock in 5 mL of 0.1% TFA/ACN (8 μg/mL). The results are typically compared to those obtained for 80 μg/mL in 50 mM Hepes buffer (pH 7.5) with 4% DMSO, using the Procedure for Hydrolysis in Buffer.

### Example 9: Preparing EphA2 Targeted Docetaxel Generating Liposomes

### Example 9A Preparation of Sucrose Octasulfate Diethylamine Salt (DEA-SOS)

Step 1 packing and conditioning: Dowex 50Wx8-200 column. Load 350 g Dowex 50WX8-200 anion exchange resin in a large column (50 mm×300 mm), wash the resin with 1200 ml 1M sodium hydroxide, 1600 ml deionized water, 1200 ml 3 M hydrochloric acid, 1600 ml deionized water consecutively.

Step 2 sucrose octasulfate (SOS) solution: dissolve 30.0 g sodium sucrose octasulfate in 15 ml deionized water in a 50-ml centrifuge tube at 50 Celsius with vigorous vortex. The solution is syringe filtered through 0.2 μm membrane.

Step 3 load SOS solution on the Dowex column prepared in step 1. Elute the column with deionized water. Collect fractions having conductivity 50˜100 mS/cm as pool A, and larger than 100 mS/cm as pool B. Immediately titrate SOS in pool B with diethylamine to a final pH of 6.7˜7.1. In case that pH of pool B pasts pH 7.1, lower the pH using the acidic SOS from pool A. SOS concentration is determined by sulfate assay and verified by the titration data.

### Example 98 Preparation of PEG-DSG-E

PEG-DSG-E is a novel conjugate of ether lipid and polyethylene glycol (PEG) designed to be less labile to the hydrolysis conditions exposed to liposomes. Due to the use of carbamate linker and ether lipid, PEG-DSG-E is more stable under mild acidic condition and prevents the loss of PEG caused by hydrolysis.

Materials: 1,2-Dioctadecyl-sn-glycerol: CAS: 82188-61-2, from BACHEM; Methoxy-PEG-NH2: Cat# 12 2000-2, from RAPP Polymere; P-Nitrophenyl Chloroformate: CAS: 7693-46-1 from Aldrich

Synthetic Procedure: PEG-DSG-E is synthesized according to the route shown in FIG. 1B. Detailed procedures are described as follows.

Step 1: Activation of 1,2-dioctadecyl-sn-glycerol. Add p-nitrophenyl chloroformate (582 mg, 2.88 mmol, 1.05 equiv.) to a solution of 1,2-dioctadecyl-sn-glycerol (1.642 g, 2.75 mmol) and triethylamine (402.5 μl, 2.89 mmol) in 35 ml dichloromethane. Stir the reaction mixture at room temperature overnight. Analyze the crude mixture (RH1:79) by TLC (Hexane/Ethyl acetate, 3/1). TLC indicates that most of starting material 1,2-dioctadecyl-sn-glycerol is converted to the activated ester RH1:79.

Step 2: Conjugation of PEG. Pour a solution of methoxy-PEG-NH2 (5 g, 2.5 mmol) in 10 ml dichloromethane into the reaction mixture of RH1:79 at room temperature. Purge the mixture with Ar and stir the mixture at room temperature overnight. Concentrate the reaction mixture to about 10 ml. Precipitate the crude product by adding 80 ml anhydrous diethyl ether with vigorous stirring. Place the mixture at −20 Celsius for 1 hour, then filter and collect the filter cake. Dissolve the filter cake in 10 ml dichloromethane and precipitate the product again from 80 ml anhydrous diethyl ether at −20 Celsius. Dissolve the filter cake in 10 ml dichloromethane and load the solution on 80 g silica gel column. Purify the crude product by flash chromatography. Mobile phase A: chloroform, B: methanol. Elution segments: step 1: 0% B˜10% B 6 CV; step 2: 10%˜15% B 2 CV. Collect all peaks detected by UV and ELSD. Fractions #18˜22 are pooled as RH1:81A; #24˜40 as RH1:81B. Yield, RH1:81A, 2.5 g. RH1:81B, 1.8 g.

TLC analysis of the reaction mixture was performed with developing solvents: chloroform/methanol, 9/1, v/v. ¹H NMR spectrum indicates that RH1:81A is the desired conjugate.

### Example 9C Preparation of Liposomes

Liposomes are prepared by ethanol injection—extrusion method. For sphingomyelin (SM) liposomes, lipids are comprised of sphingomyelin, cholesterol at the molar ratio 3:2, and PEG-DSG in the amount of 6-8 mol % of sphingomyelin. Briefly, for a 30 ml liposome preparation, lipids are dissolved in 3 ml ethanol in a 50-ml round bottom flask at 70 Celsius. DEA-SOS (27 ml, 0.65-1.1N) is warmed at 70 Celsius water bath to above 65 Celsius and mixed with the lipid solution under vigorous stirring to give a suspension having 50-100 mM phospholipid. The obtained milky mixture is then repeatedly extruded, e.g., using thermobarrel Lipex extruder (Northern Lipids, Canada) through 0.2 μm and 0.1 μm polycarbonate membranes at 65-70° C. Phospholipid concentration is measured by phosphate assay. Particle diameter is analyzed by dynamic light scattering. Liposomes prepared by this method have sizes about 95˜115 nm.

### Example 9D Loading Method for Water Soluble Drugs

Step 1: Load DEA-SOS liposome (Less than 5% of the column volume) on the Sepharose CL-4B column equilibrated with deionized water. Wait for the complete absorption of liposome, then elute the column with deionized water and monitor the conductivity of the flow-through.

Step 2: Collect liposome fractions according to the turbidity of the flow-through. Majority of the liposome come out with a conductivity of 0. Discard the tailing fractions with conductivity higher than 40 μS/cm.

Step 3: Measure the liposome volume and balance the osmolarity immediately by adding 50 wt % dextrose into liposome to obtain a final concentration of 7.5-17 wt % dextrose, depending on DEA-SOS concentration inside the liposome. Buffers are chosen from their buffering pH range and capacity. Drug loading pH should not exceed 6.

Step 4: Adjust the pH of of the liposome by using concentrated buffers, HCl, and NaOH. Final buffer strength ranges from 5 mM to 30 mM.

Step 5: Analyze the lipid concentration by phosphate assay and calculate the amount of lipids needed for given input drug/lipid ratio.

Step 6: Prepare the drug solution in 7.5-17 wt % dextrose with the same buffer as used for the liposome solution. To enable comparisons between different prodrugs, the amount of drug added was based on docetaxel weight equivalents using a conversion factor to correct from the amount of prodrug salt form weighed out (Table D).

Step 7: Mix drug and liposome solution to achieve the desired drug/phospholipid ratio (e.g., 150, 200, 300, 450, or 600 g docetaxel equivalents per mole phospholipid), then incubate at 70 Celsius (or desired temperature) for 15˜30 min with constant shaking.

Step 8: Chill the loading mixture on an ice-water bath for 15 min.

Step 9: Load part of the liposomes on a PD-10 column equilibrate with MES buffer saline (MBS) pH 5.5, or citrate buffer saline pH 5.5, or HBS pH 6.5 and eluted with the same buffer, and collect the liposomes. Keep both the purified and unpurified liposomes for next step analysis.

Step 10: Measure phospholipid concentration by phosphate assay for both before and after column samples.

Step 11: Analyze the drug concentration by HPLC for both before and after column samples.

Step 12: Encapsulation efficiency is calculated as: [drug/phospholipid (after column)]/[drug/phospholipid (before column)]*100 and described as grams of drug/mol phospholipid.

The amount of drug loaded in the liposome is expressed as docetaxel equivalents per mol phospholipid in the liposomes. To calculate number of docetaxel equivalents in a given amount of amino docetaxel hydrochloride salt, multiply the conversion factor from Table 5 below with the weighed out amount the salt. For example, 1 g of compound 1 is equivalent to 1 g×0.786=0.786 g of docetaxel. Similarly 2 g of compound 2 is equivalent to 2 g×0.819=1.638 g of Docetaxel.

TABLE 5

| | MW | MW | |
|---|---|---|---|
| | Free Base | HCl Salt | |
| Compd. | (grams/mol) | (grams/mol) | Conversion Factor |
| Docetaxel | 807.9 | | |
| 1 | 991.4 | 1027.6 | 0.786 |
| 2 | 977.2 | 1013.6 | 0.797 |
| 3 | 949.1 | 985.6 | 0.819 |
| 4 | 921.1 | 957.5 | 0.844 |
| 5 | 935.1 | 971.5 | 0.832 |
| 6 | 935.1 | 971.5 | 0.832 |

To calculate number of docetaxel equivalents in a given amount of amino docetaxel hydrochloride salt, multiply the conversion factor from Table 5 with the weighed out amount the salt. For example, 1 g of TSK-I-66 is equivalent to 1 g×0.786=0.786 g of Docetaxel. Similarly 2 g of TSK-I-47 is equivalent to 2 g×0.819=1.638 g of Docetaxel.

### Example 9E Method for Loading Drugs Poorly Soluble In Water (Less Than 1 mg/ml) By Using Short Chain Polyethylene Glycol

In this method, hexa(ethylene glycol) (PEG6) is used as an example for solubilizing and loading of drugs having very low water solubility (less than 1 mg/ml). It is believed that other short chain PEGs with molecular weight 200˜500 Daltons might also work for the same purpose. Solubilize drugs in 80% PEG6 in deionized water (by volume) at 40 mg/ml. Pre-warm liposomes to 70 Celsius and stir the solution constantly before drug loading. Then add the drug PEG6 solution to the liposome in small portions 8 times over 8 mins with 1 min interval. Incubate the loading mixture at 70 Celsius for another 22 min and cool on ice bath for 15 min. Separate free drug from liposome on a PD-10 column by using pH 5.5 MES buffer saline or pH 5.5 citrate buffer saline as the elution solution. Determine drug/lipid ratios before and after column by phosphate assay and HPLC analysis. Calculate encapsulation efficiency as: [drug/phospholipid (after column)]/[drug/phospholipid (before column)]*100

### Example 9F Method For Loading Drugs By Using PEG400

In this example, PEG400 is used to replace more expensive PEG6 as the solubilizing agent for taxane prodrugs. This method is exemplified by the protocol for preparing compound 2 liposomes.

Part 1: Preparation of Drug Solution
1. Weigh 395 mg compound 4 in a 250 ml glass bottle.
2. Add 10 ml 80% PEG400, pH 2.8 solution followed by the addition of 134 μl of 3 M HCl solution.
3. Warm the above mixture at 50° C. water bath for about 10 min with intermittent shaking. A clear solution of compound 2 is obtained.
4. Dilute the solution 10× by adding 90 ml 5 mM MES 5% dextrose pH 3.8 solution. Final solution pH is 4.5.
5. Warm up the diluted solution to 65° C., and filter the solution through 1 μm PES membrane.

Part 2: Preparation of the SOS-liposome
1. Liposome formulation: SM/Chol/PEG-DSG=3/2/0.24 mol/mol/mol, 1.1 M DEA-SOS, Size: 99.7 nm.
2. Remove the external SOS by CL-4B with residual conductivity no more than 40 μS/cm.
3. Add 45 wt % dextrose to the liposome to obtain a final 12% dextrose.
4. Add 1 M pH 5.4 citrate solution to the liposome to a final 20 mM
5. Determine the phosphate concentration by phosphate assay

Part 3: Loading of Compound 2
1. Mix compound 4 solution with liposome prepared in part 2 at drug/lipid ratio of 600 g/mol at room temperature.
2. Pump the mixture through the heat exchanger made by Teflon thin-wall tubing at 70° C. to make sure the mixture is warmed up above 65° C. within 2 min.
3. Incubate the loading mixture at 70° C. for 30 min with stirring.
4. Cool the loaded liposome rapidly by pumping them through the heat exchanger submerged in ice water.

Part 4: Purification and Concentration
1. Remove the free compound 2 by tangential flow filtration (TFF) and exchange the buffer to citrate buffer saline pH 5.5
2. Concentrate the liposome by TFF, then filter liposome through 1 μm, 0.45 μm, and 0.2 μm PES membranes sequentially.

### Example 9G Preparation of Antibody-lipid Conjugates

Antibody-PEG-lipid conjugates are used to prepare antibody-linked liposomes. They can be prepared starting with the scFv protein expressed in a convenient system (e.g. mammalian cell) and purified, e.g., by the protein A affinity chromatography, of any other suitable method. In order to effect conjugation, scFv protein is designed with a C-terminal sequence containing a cysteine residue. Preparation of scFv-PEG-lipid conjugates, such as scFv-PEG-DSPE, is described in the literature (Nellis et al. Biotechnology Progress, 2005, vol.21, p. 205-220; Nellis et al. Biotechnology Progress, 2005, vol.21, p. 221-232; U.S. Pat. No. 6,210,707). For example, the following protocol can be used:

Step 1: Dialyze the protein stock solution against pH 6.0 CES buffer (10 mM sodium citrate, 1 mM EDTA, 144 mM sodium chloride) at 4° C. for 2 h.

Step 2: Reduce the antibody in the pH 6.0 CES buffer in the presence of 20 mM 2-mercaptoethanamine at 37° C. for 1 h.

Step 3: Purify the reduced antibody on a G-25 Sephadex column.

Step 4: Incubate reduced antibody with 4 mole excess of maleimide-PEG-DSPE in pH 6.0 CES buffer at room temperature for 2 h. Quench the reaction by adding cysteine to a final concentration of 0.5 mM.

Step 5: Concentrate the conjugation mixture on an Amicon stir cell concentrator.

Step 6: Separate the conjugate from free antibody on an Ultrogel AcA44 column.

Step 7: Analyze the conjugate by SDS-PAGE.

### Example 9H Preparation of Targeted Liposomes

Antibody-targeted liposomes can be prepared by incubating antibody-PEG-lipid conjugates (Example 1G) with liposomes in an aqueous buffer at 37° C. for 12 h or at 60° C. for 30 min depending on the thermal stability of the antibody. The lipid portion of a micellar conjugate spontaneously inserts itself into the liposome bilayer. See, e.g., U.S. Pat. No. 6,210,707, incorporated by reference. The ligand inserted liposomes are purified, e.g., by size exclusion chromatography on a Sepharose CL-4B column and analyzed by phosphate assay for lipid concentration and SDS-PAGE for antibody quantification.

### Example 9I Preparation of Tritium Labeled Liposomes

Drug loaded liposomes with a non-exchangeable tritium labelled lipid, ³H-cholesterlyl hexadecyl ether (³H-CHDE) in the lipid bilayer (tritium-labeled liposomes) allow simultaneous monitoring the pharmacokinetics of both drug and lipids. Tritium-labeled liposomes of different formulations with various trapping reagents are prepared by extrusion method. The general protocol for preparing tritium-labeled “empty” liposomes (i.e., the liposomes that do not contain the drug) can be, for example, as follows.
1. Clean a 12-mL glass vial with chloroform/methanol (2/1, v/v), then acetone, and dry the vial by heat gun.
2. Transfer 1 mL commercially available ³H-CHDE solution in toluene to the glass vial. Dry the solution with a stream of argon at room temperature for 30 min, leave the vial under the oil pump vacuum overnight.
3. Weigh the lipids according to the formulation, and add them into the vial containing ³H-CHDE.
4. Add 1 mL 200-proof ethanol into the lipids vial and heat up to 70° C. to dissolve the lipids till a clear solution is obtained.
5. Add the warm lipid solution to 10 mL pre-warmed trapping reagent solution. Keep stirring the mixture at 70° C. for 15 min to produce multi-lamellar vesicles (MLV).
6. Repeatedly pass the MLVs through polycarbonate track-etched membrane filters with appropriate pore size (for example, 0.2 μm, 0.1 μm, and 0.08 μm) at 70° C. until the desired liposome size (e.g., about 110 nm) is achieved . Keep the extruded liposomes at 4° C.

Docetaxel prodrugs are loaded into tritium-labeled liposomes according to methods described in Examples 8D-F depending on drug's properties. Targeting antibody are inserted into drug loaded liposomes by the method described in Example 8H.

### Example 10: Activity of an EphA2-targeted Docetaxel Nanoliposome In Pancreatic Patient-derived Models As Monotherapy and In Combination With Gemcitabine

Pancreatic cancer remains one of the deadliest cancers with survival described in number of months and weeks. Recent advances in the treatment of pancreatic cancer led to the recent approval of a liposomal irinotecan (ONIVYDE™ (irinotecan liposome injection), previously MM-398). Given the activity of taxanes in pancreatic cancer and the ability of nanoliposomes to deliver drugs, we developed a novel EphA2-targeted nanoliposomal docetaxel (46scFv-ILs-DTXp3) and evaluated its activity in patient derived xenograft (PDX) models of pancreatic cancer as a monotherapy, as well as in combination with gemcitabine. Additionally, we aimed to test the predictive potential of key biomarkers that are linked to the 46scFv-ILs-DTXp3 mechanism of action. Several PDX models developed at Roswell Park Cancer Institute were screened for the expression of EphA2 (46scFv-ILs-DTXp3 target), CD31 (blood vessels), Massons Trichrome (fibrosis), CA XI (hypoxia), and E-Cadherin (adhesion molecule that can potentially inhibit target engagement). Eight EphA2⁺PDX models were used to evaluate the activity of 46scFv-ILs-DTXp3 and compare it to clinically relevant agents including nab-paclitaxel, liposomal irinotecan, oxaliplatin, and gemcitabine. We also tested the therapeutic potential of combined 46scFv-ILs-DTXp3 and gemcitabine.

Control of tumor growth by 46scFv-ILs-DTXp3 was statistically significant in all tested models, with tumor regression observed in more than 85% of the models. When compared with standard of care agents in tumor models, at equitoxic dosing, 46scFv-ILs-DTXp3 demonstrated greater activity to nab-paclitaxel in 80% (4/5), gemcitabine in 100% (5/5), oxaliplatin in100% (5/5), and liposomal irinotecan in 80% (4/5) of models. Gemcitabine is currently considered a standard of care in pancreatic cancer in combination with nab-paclitaxel. Thus we conducted a study to evaluate the potential benefits of combined gemcitabine and 46scFv-ILs-DTXp3. Suboptimal doses of 46scFv-ILs-DTXp3 and gemcitabine combined led to significant tumor growth control that was greater than either arm alone. Additionally, with dosing at 50% maximum tolerated dose for each agent, 46scFv-ILs-DTXp3+gemcitabine showed greater effect than nab-paclitaxel (paclitaxel protein-bound particles for injectable suspension)+gemcitabine. Although we have excluded EphA2^{negative} models from these studies, biomarker analysis showed that 46scFv-ILs-DTXp3 effects are not correlated with the EphA2 expression level, suggesting that a low level EphA2 might be sufficient to mediate activity and that liposome delivery might be the rate limiting step. Additional biomarker analysis will be conducted.

In conclusion, 46scFv-ILs-DTXp3 is highly active in several patient derived models of pancreatic cancer and its activity was equal to or greater than most standard of care agents. Future studies will aim at identifying markers for differentiating response to 46scFv-ILs-DTXp3 (EphA2 targeted nanoliposomal docetaxel) and ONIVYDE™ (irinotecan liposome injection).

We found 46scFv-ILs-DTXp3 to be highly active in tumor models derived from pancreatic patients. 46scFv-ILs-DTXp3 demonstrates superior activity compared to standard of care monotherapy, tested at two dose levels, in pancreatic PDX models. The combination of 46scFv-ILs-DTXp3 and gemcitabine was more potent than each drug alone and more potent than Gemcitabine/Nab-Paclitaxel in pancreatic PDX models.

### Example 11. Clinical Testing of 46scFv-ILs-DTXp3 Combinations

A clinical study of 46scFv-ILs-DTXp3 is conducted to evaluate the activity of MM-310 in combinations with gemcitabine or carboplatin. In Part 1, 46scFv-ILs-DTXp3 will be assessed as a monotherapy until a maximum tolerated dose (MTD) is established. Once the MTD of 46scFv-ILs-DTXp3 as a monotherapy is established, the study will proceed with Parts 2a and 2b. Part 2a of the study will assess 46scFv-ILs-DTXp3 in combination with gemcitabine in patients with urothelial carcinoma, pancreatic ductal adenocarcinoma or soft tissue sarcoma sub-types (excluding GIST). Part 2b of the study will assess 46scFv-ILs-DTXp3 in combination with carboplatin in metastatic platinum-sensitive ovarian carcinoma patients who have received two or more prior lines of therapy.

### 46scFv-ILs-DTXp3+Gemcitabine (Part 2a)

46scFv-ILs-DTXp3 will be administered IV on Day 1 of each 3-week cycle over 90 minutes. Gemcitabine will be administered IV immediately post 46scFv-ILs-DTXp3 dosing on Day 1 of each cycle over 30 minutes. A second dose of gemcitabine will be administered on Day 8 of each 3-week cycle over 30 minutes.

### 46scFv-ILs-DTXp3+Carboplatin (Part 2b)

Carboplatin will be administered IV on Day 1 of each 3-week cycle over 30 minutes. 46scFv-ILs-DTXp3 will be administered IV on Day 8 of each 3-week cycle over 90 minutes.

### Inclusion Criteria

### Part 2a

To be eligible for inclusion into the Part 2A of the study patients must have one of the following cancers, for which the patient is refractory to or intolerant to standard treatment, or for which there is no standard of care treatment available
Urothelial carcinoma
Pancreatic ductal adenocarcinoma (PDAC)
Soft tissue sarcoma subtypes except GIST, desmoid tumors and pleomorphic rhabdomyosarcoma

### Part 2b

To be eligible for inclusion into the Part 2B of the study patients must have metastatic recurrent platinum-sensitive ovarian carcinoma. Patients must have received two or more prior lines of therapy, one of which should have been a platinum-based doublet chemotherapy and they must be able to tolerate further platinum-based chemotherapy. The disease must have relapsed >6 months following most recent platinum-based chemotherapy.

### All Parts of Study

Able to provide informed consent, or have a legal representative able and willing to do so
≥18 years of age
Availability of a cancerous lesion amenable to biopsy and willing to undergo a pre-treatment biopsy
ECOG Performance Status of 0 or 1
Adequate bone marrow reserve as evidenced by:
   ANC>1,500/μl (unsupported by growth factors) and
   Platelet count>100,000/μl
   Hemoglobin>9 g/dL
Patients must have adequate coagulation function as evidenced by prothrombin time (PT), activated partial thromboplastin time (aPTT) and international normalized ratio (INR) within normal institutional limits
Adequate hepatic function as evidenced by:
   Serum total bilirubin≤ULN
   Aspartate aminotransferase (AST) and alanine aminotransferase (ALT)≤2.5×ULN.
   Alkaline phosphatase≤2.5×ULN, unless the elevated alkaline phosphatase is due to bone metastasis.
   In case alkaline phosphatase is >2.5×ULN patients are eligible for inclusion if aspartate aminotransferase (AST) and alanine aminotransferase (ALT)≤1.5×ULN
Adequate renal function as evidenced by a serum/plasma creatinine<1.5×ULN
Recovered from the effects of any prior surgery, radiotherapy or other antineoplastic therapy to CTCAE v4.03 grade 1, baseline or less, except for alopecia
Women of childbearing potential or fertile men and their partners must be willing to abstain from sexual intercourse or to use an effective form of contraception during the study and for 6 months following the last dose of 46scFv-ILs-DTXp3. Acceptable methods of effective contraception besides true abstinence include: 1) established use of oral, injected or implanted hormonal methods of contraception, 2) placement of an intrauterine device (IUD) or intrauterine system (IUS), barrier methods of contraception, including condom or occlusive cap with spermicidal foam/gel/cream/suppository, 3) male sterilization with appropriate post vasectomy documentation of the absence of sperm in the ejaculate (for female patients on the study, the vasectomized male partner should be the sole partner for that subject)

### Exclusion Criteria

### Part 2A

Prior treatment with docetaxel within 6 months of study enrollment
Prior treatment with gemcitabine within 6 months of study enrollment
Known hypersensitivity to gemcitabine

### Part 2B

Prior treatment with docetaxel-based chemotherapy
Prior treatment with a platinum-based chemotherapy

### All Parts of Study

Pregnant or lactating
Treatment with systemic anticoagulation (e.g. warfarin, heparin, low molecular weight heparin, anti-Xa inhibitors, etc.) except aspirin
Any evidence of hematemesis, melena, hematochezia, ≥grade 2 hemoptysis, or gross hematuria
Any history of hereditary bleeding disorders
Presence of an active infection or with an unexplained fever>38.5° C. during screening visits or on the first scheduled day of dosing, which in the investigator's opinion might compromise the patient's participation in the trial or affect the study outcome. At the discretion of the investigator, patients with tumor fever may be enrolled
Known CNS metastases
Known hypersensitivity to the components of 46scFv-ILs-DTXp3, or docetaxel
Prior treatment with 46scFv-ILs-DTXp3
Received treatment, within 28 days or 5 half-lives, whichever is shorter, prior to the first scheduled day of dosing, with any investigational agents that have not received regulatory approval for any indication or disease state and all prior clinically significant treatment related toxicities have resolved to Grade 1 or baseline
Received other recent antitumor therapy including any standard chemotherapy or radiation within 14 days (or have not yet recovered from any actual toxicities of the most recent therapy) prior to the first scheduled dose of 46scFv-ILs-DTXp3
Received any anti-cancer drug known to have anti-VEGF/VEGFR activity within a period of 5 half-lives of this drug (e.g. 100 days for bevacizumab, 75 days for ramucirumab) prior to the first scheduled dose of 46scFv-ILs-DTXp3
Clinically significant cardiac disease, including: NYHA Class III or IV congestive heart failure, unstable angina, acute myocardial infarction within six months of planned first dose, arrhythmia requiring therapy (including torsades de pointer, with the exception of extrasystoles, minor conduction abnormalities, or controlled and well treated chronic atrial fibrillation)
Patients who are not appropriate candidates for participation in this clinical study for any other reason as deemed by the investigator
Patients who received organ or allogeneic bone marrow or peripheral blood stem cell transplants
Chronic use of corticosteroids more than 10mg daily prednisone equivalent during the past 4 weeks prior to planned start of 46scFv-ILs-DTXp3
Concomitant use of strong inhibitors of CYP3A
Patients with peripheral neuropathy of grade 2 or higher

1. A method of treating a cancer comprising administering a therapeutically effective amount of an EphA2-targeted docetaxel-generating liposome comprising a docetaxel prodrug encapsulated within a lipid vesicle comprising one or more lipids, a PEG derivative and an EphA2 binding moiety on the outside of the lipid vesicle.

2. The method of claim 1, further comprising administering the EphA2-targeted docetaxel-generating liposome in combination with gemcitabine.

3. The method of claim 1, further comprising administering the EphA2-targeted docetaxel-generating liposome in combination with carboplatin.

4. The method of claim 1, wherein the EphA2-targeted docetaxel-generating liposome is 46scFv-ILs-DTXp3 or 46scFv-ILs-DTXp6.

5. The method of claim 1, wherein the cancer is bladder cancer or a sarcoma cancer.

6. (canceled)

7. The method of claim 5, wherein the EphA2-targeted docetaxel-generating liposome is 46scFv-ILs-DTXp3 or 46scFv-ILs-DTXp6.

8. (canceled)

9. A method of treating cancer in a human patient, the method comprising administering a therapeutically effective amount of the EphA2-targeted docetaxel-generating liposome ILs-DTXp3 or ILs-DTXp6, or administering a therapeutically effective amount of the EphA2-targeted docetaxel generating liposome 46scFv-ILs-DTXp3 or 46scFv-ILs-DTXp6, to the human patient.

10. The method of claim 9, wherein the EphA2-targeted docetaxel-generating liposome is administered in combination with gemcitabine, carboplatin, or gemcitabine and carboplatin.

11. (canceled)

12. (canceled)

13. The method of claim 1, wherein the liposome comprises sphingomyelin and cholesterol at a 3:2 molar ratio, and 5-7 mol % PEG-DSG.

14. (canceled)

15. (canceled)

16. (canceled)

17. (canceled)

18. The method of claim 1, wherein the cancer comprises cancer cells expressing an average of at least 3,000 EphA2 receptors per cell.

19. The method of claim 1, wherein the cancer comprises a cancer cell expressing an average of at least 17,500 EphA2 receptors per cell.

20. (canceled)

21. (canceled)

22. The method of claim 1, wherein the liposome encapsulates a docetaxel prodrug of Compound 3, Compound 4 or Compound 6.

23. The method of claim 1, wherein the liposome encapsulates a sucrose octasulfate salt of Compound 3, Compound 4 or Compound 6.

24. The method of claim 1, wherein the cancer is an EphA2 overexpressing cancer.

25. The method of claim 1, wherein the cancer is selected from the group consisting of a sarcoma, bladder or urothelial carcinoma, gastric, gastroesophageal junction or esophageal carcinoma (G/GEJ/E), squamous cell carcinoma of the head and neck (SCCHN), ovarian cancer, pancreatic ductal adenocarcinoma (PDAC), prostate adenocarcinoma (PAC), non-small cell lung cancer (NSCLC), small cell lung cancer (SCLC), triple negative breast cancer (TNBC), endometrial carcinoma and soft tissue sarcoma.

26. The method of claim 9, wherein the liposome comprises sphingomyelin and cholesterol at a 3:2 molar ratio, and 5-7 mol % PEG-DSG.

27. The method of claim 9, wherein the cancer comprises cancer cells expressing an average of at least 3,000 EphA2 receptors per cell.

28. The method of claim 9, wherein the liposome encapsulates a docetaxel prodrug of Compound 3, Compound 4 or Compound 6.

29. The method of claim 9, wherein the liposome encapsulates a sucrose octasulfate salt of Compound 3, Compound 4 or Compound 6.

30. The method of claim 9, wherein the cancer is an EphA2 overexpressing cancer.

31. The method of claim 9, wherein the cancer is selected from the group consisting of a sarcoma, bladder or urothelial carcinoma, gastric, gastroesophageal junction or esophageal carcinoma (G/GEJ/E), squamous cell carcinoma of the head and neck (SCCHN), ovarian cancer, pancreatic ductal adenocarcinoma (PDAC), prostate adenocarcinoma (PAC), non-small cell lung cancer (NSCLC), small cell lung cancer (SCLC), triple negative breast cancer (TNBC), endometrial carcinoma and soft tissue sarcoma.


SEQUENCE LISTING (56 sequences)

SEQ ID NO: 1 - protein, 30 aa, Artificial Sequence
  Synthetically Generated Sequence
QVQLVQSGGGLVQPGGSLRLSCAASGFTFS

SEQ ID NO: 2 - protein, 14 aa, Artificial Sequence
  Synthetically Generated Sequence
WVRQAPGKGLEWVT

SEQ ID NO: 3 - protein, 32 aa, Artificial Sequence
  Synthetically Generated Sequence
RFTISRDNSKNTLYLQMNSLRAEDTAVYYCAR

SEQ ID NO: 4 - protein, 11 aa, Artificial Sequence
  Synthetically Generated Sequence
WGQGTLVTVSS

SEQ ID NO: 5 - protein, 22 aa, Artificial Sequence
  Synthetically Generated Sequence
SSELTQPPSVSVAPGQTVTITC

SEQ ID NO: 6 - protein, 15 aa, Artificial Sequence
  Synthetically Generated Sequence
WYQQKPGTAPKLLIY

SEQ ID NO: 7 - protein, 32 aa, Artificial Sequence
  Synthetically Generated Sequence
GVPDRFSGSSSGTSASLTITGAQAEDEADYYC

SEQ ID NO: 8 - protein, 11 aa, Artificial Sequence
  Synthetically Generated Sequence
FGGGTKLTVLG

SEQ ID NO: 9 - protein, 5 aa, Artificial Sequence
  Synthetically Generated Sequence
SYAMH

SEQ ID NO: 10 - protein, 16 aa, Artificial Sequence
  Synthetically Generated Sequence
VISPAGNNTYYADSVK

SEQ ID NO: 11 - protein, 16 aa, Artificial Sequence
  Synthetically Generated Sequence
VISPAGRNKYYADSVK

SEQ ID NO: 12 - protein, 17 aa, Artificial Sequence
  Synthetically Generated Sequence
VISPDGHNTYYADSVKG

SEQ ID NO: 13 - protein, 16 aa, Artificial Sequence
  Synthetically Generated Sequence
VISPHGRNKYYADSVK

SEQ ID NO: 14 - protein, 16 aa, Artificial Sequence
  Synthetically Generated Sequence
VISRRGDNKYYADSVK

SEQ ID NO: 15 - protein, 16 aa, Artificial Sequence
  Synthetically Generated Sequence
VISNNGHNKYYADSVK

SEQ ID NO: 16 - protein, 16 aa, Artificial Sequence
  Synthetically Generated Sequence
VISPAGPNTYYADSVK

SEQ ID NO: 17 - protein, 16 aa, Artificial Sequence
  Synthetically Generated Sequence
VISPSGHNTYYADSVK

SEQ ID NO: 18 - protein, 16 aa, Artificial Sequence
  Synthetically Generated Sequence
VISPNGHNTYYADSVK

SEQ ID NO: 19 - protein, 16 aa, Artificial Sequence
  Synthetically Generated Sequence
AISPPGHNTYYADSVK

SEQ ID NO: 20 - protein, 16 aa, Artificial Sequence
  Synthetically Generated Sequence
VISPTGANTYYADSVK

SEQ ID NO: 21 - protein, 16 aa, Artificial Sequence
  Synthetically Generated Sequence
VISPHGSNKYYADSVK

SEQ ID NO: 22 - protein, 16 aa, Artificial Sequence
  Synthetically Generated Sequence
VISNNGHNTYYADSVK

SEQ ID NO: 23 - protein, 16 aa, Artificial Sequence
  Synthetically Generated Sequence
VISPAGTNTYYADSVK

SEQ ID NO: 24 - protein, 16 aa, Artificial Sequence
  Synthetically Generated Sequence
VISPPGHNTYYADSVK

SEQ ID NO: 25 - protein, 16 aa, Artificial Sequence
  Synthetically Generated Sequence
VISHDGTNTYYADSVK

SEQ ID NO: 26 - protein, 16 aa, Artificial Sequence
  Synthetically Generated Sequence
VISRHGNNKYYADSVK

SEQ ID NO: 27 - protein, 17 aa, Artificial Sequence
  Synthetically Generated Sequence
VISYDGSNKYYADSVKG

SEQ ID NO: 28 - protein, 11 aa, Artificial Sequence
  Synthetically Generated Sequence
ASVGATGPFDI

SEQ ID NO: 29 - protein, 11 aa, Artificial Sequence
  Synthetically Generated Sequence
QGDSLRSYYAS

SEQ ID NO: 30 - protein, 7 aa, Artificial Sequence
  Synthetically Generated Sequence
GENNRPS

SEQ ID NO: 31 - protein, 12 aa, Artificial Sequence
  Synthetically Generated Sequence
NSRDSSGTHLTV

SEQ ID NO: 32 - protein, 23 aa, Artificial Sequence
  Synthetically Generated Sequence
ASTGGGGSGGGGSGGGGSGGGGS

SEQ ID NO: 33 - protein, 20 aa, Artificial Sequence
  Synthetically Generated Sequence
GGGGSGGGGSGGGGSGGGGS

SEQ ID NO: 34 - protein, 15 aa, Artificial Sequence
  Synthetically Generated Sequence
GGGGSGGGGSGGGGS

SEQ ID NO: 35 - protein, 23 aa, Artificial Sequence
  Synthetically Generated Sequence
ASTGGGGAGGGGAGGGGAGGGGA

SEQ ID NO: 36 - protein, 20 aa, Artificial Sequence
  Synthetically Generated Sequence
GGGGAGGGGAGGGGAGGGGA

SEQ ID NO: 37 - protein, 24 aa, Artificial Sequence
  Synthetically Generated Sequence
TPSHNSHQVPSAGGPTANSGTSGS

SEQ ID NO: 38 - protein, 18 aa, Artificial Sequence
  Synthetically Generated Sequence
GGSSRSSSSGGGGSGGGG

SEQ ID NO: 39 - protein, 457 aa, Artificial Sequence
  Synthetically Generated Sequence
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - protein, 259 aa, Artificial Sequence
  Synthetically Generated Sequence
QVQLVQSGGGLVQPGGSLRLSCAASGFTFSSYAMHWVRQAPGKGLEWVAVISYDGSNKYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLGGGSGGC

SEQ ID NO: 41 - protein, 259 aa, Artificial Sequence
  Synthetically Generated Sequence
QVQLQQSGGGVVQPGRSLRLSCAASGFTFSSYAMHWVRQAPGKGLEWVAVISYDGSNKYYADSVKGRFTISRDNSKNTLYLQMNSLRAEDTAVYYCARASVGATGPFDIWGQGTMVTVSSASTGGGGSGGGGSGGGGSGGGGSSSELTQDPAVSVALGQTVSITCQGDSLRSYYASWYQQKPGQAPLLVIYGENNRPSGIPDRFSGSSSGNTASLTITGAQAEDEADYYCNSRDSSGTHLTVFGGGTKLTVLGGGSGGC

SEQ ID NO: 42 - protein, 214 aa, Artificial Sequence
  Synthetically Generated Sequence
DIQMTQSPSSLSASVGDRVTITCRASQSISTWLAWYQQKPGKAPKLLIYKASNLHTGVPSRFSGSGSGTEFSLTISGLQPDDFATYYCQQYNSYSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - DNA, 840 bp, Artificial Sequence
  Synthetically Generated Sequence
atgggctggtctctgatcctgctgttcctggtggccgtggccacgcgtgtgctctcgcaagtgcagctggtgcagtccggagggggactggtgcagccgggaggctcactcagactgtcctgcgccgcttcgggcttcactttctcctcgtacgctatgcattgggtccgccaagcccccggaaagggattggaatgggtggcagtgattagctacgacggctcgaacaagtactacgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggtggcggcagcggcggctgctgataa

SEQ ID NO: 44 - protein, 259 aa, Artificial Sequence
  Synthetically Generated Sequence
QVQLVQSGGGLVQPGGSLRLSCAASGFTFSSYAMHWVRQAPGKGLEWVSVISPAGRNKYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLGGGSGGC

SEQ ID NO: 45 - DNA, 840 bp, Artificial Sequence
  Synthetically Generated Sequence
atgggctggtctctgatcctgctgttcctggtggccgtggccacgcgtgtgctctcgcaagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgagcgtgattagcccggcgggccgcaacaaatattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggtggcggcagcggcggctgctgataa

SEQ ID NO: 46 - protein, 259 aa, Artificial Sequence
  Synthetically Generated Sequence
QVQLVQSGGGLVQPGGSLRLSCAASGFTFSSYAMHWVRQAPGKGLEWVTVISPDGHNTYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLGGGSGGC

SEQ ID NO: 47 - DNA, 861 bp, Artificial Sequence
  Synthetically Generated Sequence
atgggctggtctctgatcctgctgttcctggtggccgtggccacgcgtgtgctctcgcaagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgaccgtgattagcccggatggccataacacctattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggtcgtacggtggcggcgcccagtcaccatcatcatcaccactgataa

SEQ ID NO: 48 - protein, 259 aa, Artificial Sequence
  Synthetically Generated Sequence
QVQLVQSGGGLVQPGGSLRLSCAASGFTFSSYAMHWVRQAPGKGLEWVTVISPSGHNTYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLGGGSGGC

SEQ ID NO: 49 - DNA, 840 bp, Artificial Sequence
  Snythetically Generated Sequence
atgggctggtctctgatcctgctgttcctggtggccgtggccacgcgtgtgctctcgcaagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgaccgtgattagcccgagcggccataacacctattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggtggcggcagcggcggctgctgataa

SEQ ID NO: 50 - protein, 259 aa, Artificial Sequence
  Synthetically Generated Sequence
QVQLVQSGGGLVQPGGSLRLSCAASGFTFSSYAMHWVRQAPGKGLEWVTVISPNGHNTYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLGGGSGGC

SEQ ID NO: 51 - DNA, 840 bp, Artificial Sequence
  Synthetically Generated Sequence
atgggctggtctctgatcctgctgttcctggtggccgtggccacgcgtgtgctctcgcaagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgaccgtgattagcccgaacggccataacacctattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggtggcggcagcggcggctgctgataa

SEQ ID NO: 52 - protein, 259 aa, Artificial Sequence
  Synthetically Generated Sequence
QVQLVQSGGGLVQPGGSLRLSCAASGFTFSSYAMHWVRQAPGKGLEWVSAISPPGHNTYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLGGGSGGC

SEQ ID NO: 53 - DNA, 816 bp, Artificial Sequence
  Synthetically Generated Sequence
atgggctggtctctgatcctgctgttcctggtggccgtggccacgcgtgtgctctcgcaagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgagcgcgattagcccgccgggccataacacctattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggt

SEQ ID NO: 54 - protein, 259 aa, Artificial Sequence
  Synthetically Generated Sequence
QVQLVQSGGGLVQPGGSLRLSCAASGFTFSSYAMHWVRQAPGKGLEWVTVISPTGANTYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLGGGSGGC

SEQ ID NO: 55 - DNA, 840 bp, Artificial Sequence
  Synthetically Generated Sequence
atgggctggtctctgatcctgctgttcctggtggccgtggccacgcgtgtgctctcgcaagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgaccgtgattagcccgaccggcgcgaacacctattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggtggcggcagcggcggctgctgataa

SEQ ID NO: 56 - DNA, 840 bp, Artificial Sequence
  Synthetically Generated Sequence
atgggctggtctctgatcctgctgttcctggtggccgtggccacgcgtgtgctctcgcaagtgcagctgcagcagtccggagggggagtggtgcagccgggacggtcactcagactgtcctgcgccgcttcgggcttcactttctcctcgtacgctatgcattgggtccgccaagcccccggaaagggattggaatgggtggcagtgattagctacgacggctcgaacaagtactacgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccatggtcaccgtgtcatcggcatcgactggagggggaggctctggaggagggggatcgggtggcggagggtcgggcggaggaggctcatcatccgagttgacccaagatccggccgtgtccgtggcgctggggcagactgtctccatcacttgccaaggagactcactgcgctcctactacgcctcgtggtatcagcagaaaccgggacaggctcctctgctcgtgatctacggcgaaaacaatcggccatcgggaatccctgaccgctttagcggttcgagctccggaaacactgcgagcctgaccatcactggtgcccaagccgaggatgaagcggactactactgcaactcgcgggattcctccgggacccacctgaccgtgttcggcgggggaactaagctgaccgtgctgggtggcggcagcggcggctgctgataa